SEQUENCE LISTING (65 sequences)

SEQ ID NO: 1 - DNA, 6227 bp, Artificial Sequence
  Activation plasmid dgRNA-MS2MPH
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctaggccaacatgaggatcacccatgtctgcagggcctagcaagttaaaataaggctagtccgttatcaacttggccaacatgaggatcacccatgtctgcagggccaagtggcaccgagtcggtgctttttggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttagtctagaatgcccaaaaagaaaagaaaagtgggtagtatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacggaggaggtggaagcggaggaggaggaagcggaggaggaggtagcctcgagggacctaagaaaaagaggaaggtggcggccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaggacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcctgagggcccaacttgtttattgcagcttataatggttacaaataaagcaatagcatcacaaatttcacaaataaagcatttttttcactgcattctagttgtggtttgtccaaactcatcaatgtatcttagtcgacgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 4193 bp, Artificial Sequence
  Repression plasmid dgRNA-ComCK
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgtttaagagctatgctggaaacagcatagcaagtttaaataaggctagtccgttatcaacttgaaaaagtggcaccgagtcggtgcctgaatgcctgcgagcatctttttttgttttttatgtctggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttagtctagaatgcccaaaaagaaaagaaaagtgggtagtatgaaatcaattcgctgtaaaaactgcaacaaactgttatttaaggcggatagttttgatcacattgaaatcaggtgtccgcgttgcaaacgtcacatcataatgctgaatgcctgcgagcatcccacggagaaacattgtgggaaaagagaaaaaatcacgcattctgacgaaaccgtgcgttatggaggaggtggaagcggaggaggaggaagcggaggaggaggtagcctcgagatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctaggggcccaacttgtttattgcagcttataatggttacaaataaagcaatagcatcacaaatttcacaaataaagcatttttttcactgcattctagttgtggtttgtccaaactcatcaatgtatcttagtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 3 - DNA, 14 bp, Artificial Sequence
  dgRNA
gcgctctagccacc

SEQ ID NO: 4 - DNA, 14 bp, Artificial Sequence
  dgRNA
acgggctacccgat

SEQ ID NO: 5 - DNA, 15 bp, Artificial Sequence
  dgRNA
gcgctcgcactcagt

SEQ ID NO: 6 - DNA, 14 bp, Artificial Sequence
  dgRNA
ctagtcagcggagc

SEQ ID NO: 7 - DNA, 15 bp, Artificial Sequence
  dgRNA
gaactgtgccaatgc

SEQ ID NO: 8 - DNA, 14 bp, Artificial Sequence
  dgRNA
gcgtgacgtcgcgc

SEQ ID NO: 9 - DNA, 15 bp, Artificial Sequence
  dgRNA
gggcagctggaggaa

SEQ ID NO: 10 - DNA, 15 bp, Artificial Sequence
  dgRNA
atcgccctccgggat

SEQ ID NO: 11 - DNA, 15 bp, Artificial Sequence
  dgRNA
gtcgccagactcttc

SEQ ID NO: 12 - DNA, 14 bp, Artificial Sequence
  dgRNA
gcatcaagcccttg

SEQ ID NO: 13 - DNA, 14 bp, Artificial Sequence
  dgRNA
ggcccttaaaactt

SEQ ID NO: 14 - DNA, 14 bp, Artificial Sequence
  dgRNA
acacttcagtgcac

SEQ ID NO: 15 - DNA, 14 bp, Artificial Sequence
  dgRNA
tacctcggcggcgt

SEQ ID NO: 16 - DNA, 15 bp, Artificial Sequence
  dgRNA
gagcccccgcgacgg

SEQ ID NO: 17 - DNA, 15 bp, Artificial Sequence
  dgRNA
ggggctcactggcag

SEQ ID NO: 18 - DNA, 15 bp, Artificial Sequence
  dgRNA
ggtagaagctggttg

SEQ ID NO: 19 - DNA, 14 bp, Artificial Sequence
  dgRNA
gttggctttcgtca

SEQ ID NO: 20 - DNA, 15 bp, Artificial Sequence
  dgRNA
gcatgctcagagttc

SEQ ID NO: 21 - DNA, 16 bp, Artificial Sequence
  dgRNA
gcctttcaggcctagc

SEQ ID NO: 22 - DNA, 16 bp, Artificial Sequence
  dgRNA
gtactagcgtttcagg

SEQ ID NO: 23 - DNA, 14 bp, Artificial Sequence
  dgRNA
gctcgctgcagcag

SEQ ID NO: 24 - DNA, 15 bp, Artificial Sequence
  dgRNA
gaggccaggggccgg

SEQ ID NO: 25 - DNA, 14 bp, Artificial Sequence
  dgRNA
attagtcagcaacc

SEQ ID NO: 26 - DNA, 15 bp, Artificial Sequence
  dgRNA
actgggcggagttag

SEQ ID NO: 27 - DNA, 14 bp, Artificial Sequence
  dgRNA
ggccgaggccgcct

SEQ ID NO: 28 - DNA, 14 bp, Artificial Sequence
  dgRNA
cagaagtagtgagg

SEQ ID NO: 29 - DNA, 8434 bp, Artificial Sequence
  TRE-MPH
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaaacccagctttcttgtacaaagtggtgatcttaaggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctaggccaacatgaggatcacccatgtctgcagggcctagcaagttaaaataaggctagtccgttatcaacttggccaacatgaggatcacccatgtctgcagggccaagtggcaccgagtcggtgctttttcggatcccgatcacgagactagcctcgagttggctttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaaccgcggccccgaattgcaagtttgtacaaaggtaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacggaggaggtggaagcggaggaggaggaagcggaggaggaggtagcctcgagggacctaagaaaaagaggaaggtggcggccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaggacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcctgagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 30 - DNA, 7482 bp, Artificial Sequence
  TRE-CK
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaaacccagctttcttgtacaaagtggtgatcttaaggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgtttaagagctatgctggaaacagcatagcaagtttaaataaggctagtccgttatcaacttgaaaaagtggcaccgagtcggtgcctgaatgcctgcgagcatctttttttgttttttatgtctcggatcccgatcacgagactagcctcgagttggctttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaaccgcggccccgaattgcaagtttgtacaaaggtaccatgcccaaaaagaaaagaaaagtgggtagtatgaaatcaattcgctgtaaaaactgcaacaaactgttatttaaggcggatagttttgatcacattgaaatcaggtgtccgcgttgcaaacgtcacatcataatgctgaatgcctgcgagcatcccacggagaaacattgtgggaaaagagaaaaaatcacgcattctgacgaaaccgtgcgttatggaggaggtggaagcggaggaggaggaagcggaggaggaggtagcctcgagatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctaggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 31 - DNA, 1508 bp, Artificial Sequence
  TLR
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaacctgcagggagcagcgtcttcgagagtgaggacactagtgtgaaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattccggagggcagaggaagtctgctaacatgcggtgacgtcgaggagaatcctggcccaggatccgtgagcaagggcgaggaggataactccgccatcatcaaggagttcctgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctctctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtga

SEQ ID NO: 32 - DNA, 5456 bp, Artificial Sequence
  Traffic light plasmid
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaacctgcagggagcagcgtcttcgagagtgaggacactagtgtgaaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattccggagggcagaggaagtctgctaacatgcggtgacgtcgaggagaatcctggcccaggatccgtgagcaagggcgaggaggataactccgccatcatcaaggagttcctgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctctctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 33 - DNA, 2319 bp, Artificial Sequence
  AAVS1 HDR donor DNA sequence
ttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggggtaccagtcgatccaacatggcgacttgtcccatccccggcatgtttaaatatactaattattcttgaactaattttaatcaaccgatttatctctcttccgcaggtggcggaggttccggtggaagcggaggtagcggcggatccgagggccgcggcagcctgctgacctgcggcgatgtggaggagaaccccgggcccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaataaaagatctttattttcattagatctgtgtgttggttttttgtgtgaattcccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgt

SEQ ID NO: 34 - DNA, 2100 bp, Artificial Sequence
  ACTB HDR donor DNA sequence
cggctctgcctgacatgagggttacccctcggggctgtgctgtggaagctaagtcctgccctcatttccctctcaggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaaggtgggtgtctttcctgcctgagctgacctgggcaggtcggctgtggggtcctgtggtgtgtggggagctgtcacatccagggtcctcactgcctgtccccttccctcctcagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttcgagggccgcggcagcctgctgacctgcggcgatgtggaggagaaccccgggcccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaggccaagtgtgactttgtggtgtggctgggttgggggcagcagagggtgaaccctgcaggagggtgaaccctgcaaaagggtggggcagtgggggccaacttgtccttacccagagtgcaggtgtgtggagatccctcctgccttgacattgagcagccttagagggtgggggaggctcaggggtcaggtctctgttcctgcttattgggga

SEQ ID NO: 35 - DNA, 4333 bp, Artificial Sequence
  sgVenus-ECFP expression plasmid
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacgggatccgatggaacactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttctcgagggaacatctagatgcattcgcgaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 36 - DNA, 3656 bp, Artificial Sequence
  Actb-F-T2A-GFP-R
atggatgatgatatcgccgcgctcgtcgtcgacaacggctccggcatgtgcaaggccggcttcgcgggcgacgatgccccccgggccgtcttcccctccatcgtggggcgccccaggcaccaggtaggggagctggctgggtggggcagccccgggagcgggcgggaggcaagggcgctttctctgcacaggagcctcccggtttccggggtgggggctgcgcccgtgctcagggcttcttgtcctttccttcccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccaggtgagtggcccgctacctcttctggtggccgcctccctccttcctggcctcccggagctgcgccctttctcactggttctctcttctgccgttttccgtaggactctcttctctgacctgagtctcctttggaactctgcaggttctatttgctttttcccagatgagctctttttctggtgtttgtctctctgactaggtgtctaagacagtgttgtgggtgtaggtactaacactggctcgtgtgacaaggccatgaggctggtgtaaagcggccttggagtgtgtattaagtaggtgcacagtaggtctgaacagactccccatcccaagaccccagcacacttagccgtgttctttgcactttctgcatgtcccccgtctggcctggctgtccccagtggcttccccagtgtgacatggtgtatctctgccttacagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggtgagtggagactgtctcccggctctgcctgacatgagggttacccctcggggctgtgctgtggaagctaagtcctgccctcatttccctctcaggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaaggtgggtgtctttcctgcctgagctgacctgggcaggtcggctgtggggtcctgtggtgtgtggggagctgtcacatccagggtcctcactgcctgtccccttccctcctcagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttcgagggccgcggcagcctgctgacctgcggcgatgtggaggagaaccccgggcccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaggccaagtgtgactttgtggtgtggctgggttgggggcagcagagggtgaaccctgcaggagggtgaaccctgcaaaagggtggggcagtgggggccaacttgtccttacccagagtgcaggtgtgtggagatccctcctgccttgacattgagcagccttagagggtgggggaggctcaggggtcaggtctctgttcctgcttattggggagttcctggcctggcccttctatgtctccccaggtaccccagtttttctgggttcacccagagtgcagatgcttgaggaggtgggaagggactatttgggggtgtctggctcaggtgccatgcctcactggggctggttggcacctgcatttcctgggagt

SEQ ID NO: 37 - DNA, 7634 bp, Artificial Sequence
  SA-T2A-EGFP
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgggttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggggtaccagtcgatccaacatggcgacttgtcccatccccggcatgtttaaatatactaattattcttgaactaattttaatcaaccgatttatctctcttccgcaggtggcggaggttccggtggaagcggaggtagcggcggatccgagggccgcggcagcctgctgacctgcggcgatgtggaggagaaccccgggcccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattcccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgtctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 38 - DNA, 8771 bp, Artificial Sequence
  Lentivirus expressing dgCDK1-MS2MPH
ttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacgggataccgtctctgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttggatccaagcttggcgtaactagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgagggggcccggggaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtatgaaaaagcctgaactcaccgctacctctgtcgagaagtttctgatcgaaaagttcgacagcgtgtccgacctgatgcagctctccgagggcgaagaatctcgggctttcagcttcgatgtgggagggcgtggatatgtcctgcgggtgaatagctgcgccgatggtttctacaaagatcgctatgtttatcggcactttgcatccgccgctctccctattcccgaagtgcttgacattggggagttcagcgagagcctgacctattgcatctcccgccgtgcacagggtgtcaccttgcaagacctgcctgaaaccgaactgcccgctgttctccagcccgtcgccgaggccatggatgccatcgctgccgccgatcttagccagaccagcgggttcggcccattcggacctcaaggaatcggtcaatacactacatggcgcgatttcatctgcgctattgctgatccccatgtgtatcactggcaaactgtgatggacgacaccgtcagtgcctccgtcgcccaggctctcgatgagctgatgctttgggccgaggactgccccgaagtccggcacctcgtgcacgccgatttcggctccaacaatgtcctgaccgacaatggccgcataacagccgtcattgactggagcgaggccatgttcggggattcccaatacgaggtcgccaacatcttcttctggaggccctggttggcttgtatggagcagcagacccgctacttcgagcggaggcatcccgagcttgcaggatctcctcggctccgggcttatatgctccgcattggtcttgaccaactctatcagagcttggttgacggcaatttcgatgatgcagcttgggctcagggtcgctgcgacgcaatcgtccggtccggagccgggactgtcgggcgtacacaaatcgcccgcagaagcgctgccgtctggaccgatggctgtgtggaagtgctcgccgatagtggaaacagacgccccagcactcgtcctagggcaaagggcagtggagagggcagaggaagtctgctaacatgcggtgacgtcgaggagaatcctggcccaatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcggccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcctaatgtacaagcgctaataaaagatctttattttcattagatctgtgtgttggttttttgtgtggtaactctagacgtgcggtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagc

SEQ ID NO: 39 - DNA, 22 bp, Artificial Sequence
  primer
tgacctacggcgtgcagtgctt

SEQ ID NO: 40 - DNA, 21 bp, Artificial Sequence
  primer
cctcgaacttcacctcggcgc

SEQ ID NO: 41 - DNA, 22 bp, Artificial Sequence
  primer
tttggtcgtattgggcgcctgg

SEQ ID NO: 42 - DNA, 22 bp, Artificial Sequence
  primer
ctcagccttgacggtgccatgg

SEQ ID NO: 43 - DNA, 22 bp, Artificial Sequence
  primer
gaggagcaggagcttctcgact

SEQ ID NO: 44 - DNA, 22 bp, Artificial Sequence
  primer
aacgccactgacaagaaagcac

SEQ ID NO: 45 - DNA, 22 bp, Artificial Sequence
  primer
ggctactatcacaagcctgtgg

SEQ ID NO: 46 - DNA, 21 bp, Artificial Sequence
  primer
ttgcccatgatggcagaggca

SEQ ID NO: 47 - DNA, 23 bp, Artificial Sequence
  primer
ctacaggtcaagtggtagccatg

SEQ ID NO: 48 - DNA, 24 bp, Artificial Sequence
  primer
ctggaatcctgcataagcacatcc

SEQ ID NO: 49 - DNA, 21 bp, Artificial Sequence
  primer
caacagctgagggaacagcag

SEQ ID NO: 50 - DNA, 21 bp, Artificial Sequence
  primer
agtttaagatcctgctgccgg

SEQ ID NO: 51 - DNA, 20 bp, Artificial Sequence
  primer
ggtaaaggatcacggggtgg

SEQ ID NO: 52 - DNA, 20 bp, Artificial Sequence
  primer
gctgcttggtggagcttttc

SEQ ID NO: 53 - DNA, 23 bp, Artificial Sequence
  primer
ggctgtggtgttctatggtaccg

SEQ ID NO: 54 - DNA, 22 bp, Artificial Sequence
  primer
ccgtggcccatcatgtcttgga

SEQ ID NO: 55 - DNA, 22 bp, Artificial Sequence
  primer
gttgtgctgtgtatggacgtgg

SEQ ID NO: 56 - DNA, 23 bp, Artificial Sequence
  primer
gtgccatcagtaccaaacaggac

SEQ ID NO: 57 - DNA, 26 bp, Artificial Sequence
  primer
agatctatggtgagcaagggcgagga

SEQ ID NO: 58 - DNA, 28 bp, Artificial Sequence
  primer
gaattcttacttgtacagctcgtccatg

SEQ ID NO: 59 - DNA, 19 bp, Artificial Sequence
  primer
gggtcacctctacggctgg

SEQ ID NO: 60 - DNA, 27 bp, Artificial Sequence
  primer
cgaattcttacttgtacagctcgtcca

SEQ ID NO: 61 - DNA, 20 bp, Artificial Sequence
  sgRNA target
gagcagcgtcttcgagagtg

SEQ ID NO: 62 - DNA, 20 bp, Artificial Sequence
  sgRNA target
caccccacagtggggccact

SEQ ID NO: 63 - DNA, 20 bp, Artificial Sequence
  sgRNA target
tgtccctagtggccccactg

SEQ ID NO: 64 - DNA, 20 bp, Artificial Sequence
  sgRNA target
ccaccgcaaatgcttctagg

SEQ ID NO: 65 - DNA, 4383 bp, Artificial Sequence
  sgAAVS1-mCherry plasmid
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagtcgacgggcccgggatccgatggaacactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttctcgagggaacatctagatgcattcgcgaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

(19) United States
(12) Patent Application Publication
(10) Pub. No.: US 2021/0130818 A1
(43) Pub. Date: May 6, 2021

(54) Compositions and Methods for Enhancement of Homology-Directed Repair Mediated Precise Gene Editing by Programming DNA Repair with a Single RNA-Guided Endonuclease

(71) Applicants: YALE UNIVERSITY, New Haven, CT (US); ZHEJIANG UNIVERSITY, Xihu District, Hangzhou (CN)

(72) Inventors: Sidi Chen, Milford, CT (US); Lupeng Ye, New Haven, CT (US); Chengkun Wang, Tai'An (CN); Feng Han, Hangzhou (CN)

(21) Appl. No.: 17/257,476

(22) PCT Filed: Jul. 4, 2018
(86) PCT No.: PCT/CN2018/094499

§ 371 Date: Dec. 31, 2020

(51) Int. Cl. C12N 15/11; C12N 15/90; C12N 9/22; C12N 15/86; C12N 7/00

(52) U.S. Cl. CPC C12N 15/11; C12N 15/907; C12N 9/22; C12N 15/86; C12N 2830/003; C12N 2310/20; C12N 2830/50; C12N 2800/80; C12N 2740/15043; C12N 7/00

(57) ABSTRACT

The present invention includes compositions and methods for enhancing homology directed repair (HDR) and/or reducing non-homologous end joining (NHEJ) in a cell following CRISPR-mediated editing.

### STATEMENT REGARDING FEDERALLY SPONSORED RESEARCH OR DEVELOPMENT

This invention was made with government support under CA209992 awarded by the National Institutes of Health. The government has certain rights in the invention.

### BACKGROUND OF THE INVENTION

Organisms have evolved multiple mechanisms to maintain genome integrity. As the cellular genome is constantly exposed to environmental damage, multiple DNA damage repair pathways exist to protect the genome from harmful or potentially catastrophic alterations. Double-strand break (DSB) repair pathways are highly conserved between eukaryotes including mammalian species. Non-homologous DNA end-joining (NHEJ) and homologous-directed recombination (HDR) are two major DNA repair pathways that can either act in concert or antagonistic manner. HDR is a pathway which uses template DNA such as an intact sister chromosomal copy or an exogenous donor to repair the DSBs, and thus can robustly generate perfect repair. However, HDR efficiency depends on species, cell type and the stage of the cell cycles. In mammalian cells, NHEJ has been considered the major pathway to repair the DNA, whereas HDR is more common in Saccharomyces cerevisiae. NHEJ is an imperfect process, which often leads to gain or loss of a few nucleotides at each end of the breakage site. This character can lead to subsequent deleterious genetic alteration that results in cellular malfunctioning, cancer or aging. The DNA repair enzymes KU70, KU80, and Ligase IV (LIG4) play central roles in NHEJ-mediated DNA repair, whereas KU70 and KU80 proteins stabilize the DNA ends and put them in physical proximity to facilitate end ligation performed by LIG4. On the other hand, proteins such as BRCA1/2, RAD50, RAD51 and various cell cycle regulators are directly involved in HDR, although the pathway has yet to be fully characterized.

The type II bacterial adaptive immune system, clustered regularly interspaced palindromic repeats (CRISPR)-associated protein 9 (Cas9) is a powerful genome editing tool. The Cas9-single guide RNA (sgRNA) complex induces site-specific DSBs, which can be repaired by either of the two main DNA repair pathways, NHEJ and HDR. The error-prone repairs by NHEJ often introduce unpredictable frame shift insertions and deletions (indels), leading to loss-of-function of target genes. In contrast, HDR can either generate perfect DNA repair or precise genome modification guided by donor templates. However, HDR is substantially less efficient compared to NHEJ in mammalian cells and most often restricted to S/G2 phase(s) of the cell cycle. Owning to the importance of HDR in mediating precise genetic modification, extensive efforts have been made to change the balance of DNA repair pathways. However, due to the intricacy of the DNA repair pathways, the available tools to enhance HDR are still limited to a few choices with relatively small effect. Moreover, little success to date has been achieved to directly augment the HDR pathway itself. Thus, manipulation of both HDR and NHEJ using simple genetic tools might enable or strengthen a variety of genome editing applications.

A need exists for compositions and methods for enhancing HDR. The present invention satisfies this need.

### SUMMARY OF THE INVENTION

As described herein, the present invention relates to compositions and methods for enhancing homology directed repair (HDR) and/or decreasing DNA non-homologous end-joining (NHEJ) following CRISPR editing in a cell.

One aspect of the invention includes a vector comprising a first promoter, a dead guide RNA (dgRNA) comprising a 14-15 base pair (bp) sequence that targets a homology directed repair (HDR) gene and two MS2 binding loops, a second promoter, an MCP sequence, and a P65-HSF1 sequence.

Another aspect of the invention includes a vector comprising a first promoter, a dgRNA comprising a 14-15 base pair (bp) sequence that targets a non-homologous end joining (NHEJ) gene and a Com binding loop, a second promoter, a Com sequence, and a KRAB sequence.

Yet another aspect of the invention includes a vector comprising a promoter, a nonfunctional green fluorescent reporter containing a CRISPR targeting site, a self cleaving peptide, and a red fluorescent reporter containing a 2-bp shifted reading frame.

Still another aspect of the invention includes a vector comprising a first promoter, an rtTA sequence, a second promoter, a dead guide RNA (dgRNA) comprising a 14-15 base pair (bp) sequence that targets a homology directed repair (HDR) gene and two MS2 binding loops, a TREG3G promoter sequence, an MCP sequence, and a P65-HSF1 sequence.

In one aspect, the invention includes a vector comprising a first promoter sequence, an rtTA sequence, a second promoter, a dgRNA comprising a 14-15 base pair (bp) sequence that targets a non-homologous end joining (NHEJ) gene and a COM binding loop, a TREG3G promoter sequence, a COM sequence, and KRAB sequence.

In another aspect, the invention includes a vector comprising a first promoter, a dgRNA comprising a CDK1-2 targeting sequence and and two MS2 binding loops, a second promoter, an MCP sequence, and a P65-HSF1 sequence.

In yet another aspect, the invention includes a composition comprising any vector of the present invention and a Cas9.

In still another aspect, the invention includes a cell comprising one or more of the vectors of the present invention.

Another aspect of the invention includes a method of enhancing homology directed repair (HDR) and/or decreasing DNA non-homologous end-joining (NHEJ) following CRISPR editing in a cell. The method comprises administering to the cell a Cas9, a sgRNA, an activation plasmid, and a HDR donor template. The activation plasmid comprises a first promoter, a dead guide RNA (dgRNA) comprising a 14-15 base pair (bp) sequence that targets a homology directed repair (HDR) gene and two MS2 binding loops, a second promoter, an MCP sequence, and a P65-HSF1 sequence.

Yet another aspect of the invention includes a method of enhancing homology directed repair (HDR) and/or decreasing DNA non-homologous end-joining (NHEJ) following CRISPR editing in a cell, comprising administering to the cell a Cas9, a sgRNA, a repression plasmid, and a HDR donor template. The repression plasmid comprises a first promoter, a dgRNA comprising a 14-15 base pair (bp) sequence that targets a non-homologous end joining (NHEJ) gene and a Com binding loop, a second promoter, a Com sequence, and KRAB sequence.

Still another aspect of the invention includes a method of enhancing homology directed repair (HDR) and/or decreasing DNA non-homologous end-joining (NHEJ) following CRISPR editing in a cell, comprising administering to the cell a Cas9, a sgRNA, an activation plasmid, a repression plasmid, and a HDR donor template. The activation plasmid comprises a first promoter, a dead guide RNA (dgRNA) comprising a 14-15 base pair (bp) sequence that targets a homology directed repair (HDR) gene and two MS2 binding loops, a second promoter, an MCP sequence, and a P65-HSF1 sequence. The repression plasmid comprises a first promoter, a dgRNA comprising a 14-15 base pair (bp) sequence that targets a non-homologous end joining (NHEJ) gene and a Com binding loop, a second promoter, a Com sequence, and KRAB sequence.

In another aspect, the invention includes a composition comprising two of the vectors of the present invention.

In yet another aspect, the invention includes a kit comprising two of the vectors of the present invention, and instructional material for use thereof.

In various embodiments of the above aspects or any other aspect of the invention delineated herein, the vector comprises SEQ ID NO: 1. In one embodiment, the vector comprises SEQ ID NO: 2. In one embodiment, the vector comprises SEQ ID NO: 29. In one embodiment, the vector comprises SEQ ID NO: 30. In one embodiment, the vector comprises the nucleotide sequence of SEQ ID NO: 31 or SEQ ID NO: 32. In one embodiment, the vector comprises SEQ ID NO: 38.

In one embodiment, the HDR gene is selected from the group consisting of CDK1, CtIP, BRCA1/2, RAD50, and RAD51. In one embodiment, the sequence that targets a HDR gene is selected from the group consisting of SEQ ID NOs: 3-12. In one embodiment, the NHEJ gene is selected from the group consisting of LIG4, KU70 and KU80. In one embodiment, the NHEJ sequence is selected from the group consisting of SEQ ID NOs. 13-22.

In one embodiment, the first promoter comprises a CMV promoter or a U6 promoter and the second promoter comprises a CMV promoter or a U6 promoter. In one embodiment, the promoter is a CMV promoter. In one embodiment, the vector further comprises at least one component selected from the group consisting of an NLS sequence, a linker sequence, a polyA sequence, an SV40 sequence, and an antibiotic resistance sequence. In one embodiment, the vector further comprises a SV40 poly (A) signal.

In one embodiment, the nonfunctional green fluorescent reporter comprises an EGFP variant wherein codons 53-63 are disrupted.

In one embodiment, the cell is a human embryonic kidney 293 (HEK293) cell. In one embodiment, the cell further comprises a Cas9.

In one embodiment, the vector comprises a lentiviral backbone.

In one embodiment, the activation plasmid targets CDK1-2 and/or the repression plasmid targets KU80-1. In one embodiment, the repression and/or activation plasmid further comprises an inducible expression system. In one embodiment, the inducible expression system is a Tet-On system inducible by doxycycline (Dox).

In one embodiment, the activation plasmid comprises SEQ ID NO: 1. In one embodiment, the repression plasmid comprises SEQ ID NO: 2. In one embodiment, the first promoter of the repression and/or activation plasmid comprises a CMV promoter or a U6 promoter and the second promoter of the repression and/or activation plasmid comprises a CMV promoter or a U6 promoter. In one embodiment, the repression and/or activation plasmid further comprises at least one component selected from the group consisting of an NLS sequence, a linker sequence, a polyA sequence, an SV40 sequence, and an antibiotic resistance sequence.

In one embodiment, any method of the present invention further comprises administering the cell to an animal. In one embodiment, the repression and/or activation plasmid is packaged into a lentiviral vector. In one embodiment, the method further comprises administering the lentiviral vector to an animal. In one embodiment, the animal is a human.

In one embodiment, the composition further comprises a Cas9. In one embodiment, the kit further comprises a Cas9.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIGS. 1A-1L illustrate the finding that programming key genes of HDR and NHEJ pathways enhances HDR efficiency. FIG. 1A is a diagram of the dgRNA-MS2:MPH expression vector for activating key genes of the HDR pathway. FIG. 1B is a diagram of the dgRNA-Com:CK expression vector for repressing key genes of the NHEJ pathway.

FIG. 1C is a diagram of the TLR system. Cas9/sgRNA can induce DSBs in the target site. If DSBs are repaired by NHEJ, 3n+2 bp frame shift indels can restore mCherry expression, which accounts for approximately 1/3 of the mutagenic NHEJ events. Alternatively, if DSBs are repaired yielding an intact EGFP template, the mutations in bf-Venus will be corrected, leading to Venus (EGFP variant) expression. FIG. 1D shows quantitative results of HDR efficiency by programming essential components of DNA repair pathways. FIG. 1E shows a strategy for insertion of an EGFP reporter gene into the human AAVS1 locus using CRISPR-Cas9 in human cells. The SA-T2A-EGFP promoterless cassette was flanked by two AAVS1 homology arms, left arm (489 bp) and right arm (855 bp). SA, splice acceptor; T2A, a self-cleaving peptide; PA, a short polyA signal; primer F and primer R were designed for EGFP-positive events identification and sequencing. FIG. 1F shows chromatogram and sequences of HDR-positive events. Partial donor sequences and adjacent genomic DNA sequence are represented. FIGS. 1G-1L show HDR efficiency determined in three different cell lines, HEK293, HEK293T and HeLa. CDK1 activation and/or KU80 repression significantly increased HDR efficiency. These cell lines were co-transfected with SA-T2A-EGFP donor and sgAAVS1-mCherry expression vectors 24 h after dgRNA-Com:CK and/or dgRNA-MS2:MPH transfection. At day 3, the frequency of EGFP⁺ cells within mCherry⁺ population were determined using FACS. Data are showed as the mean±SD from three independent experiments. Significance was calculated using the Paired t test. * P<0.05, ** P<0.01, *** P<0.001.

FIGS. 2A-2F illustrates the finding that activating CDK1 and repressing KU80 enhances HDR efficiency in endogenous loci. FIG. 2A is a schematic of an insertion strategy at the human AAVS1 locus. A new AAVS1 targeting site was designed, sgAAVS1-2 was close to the sgAAVS1-1 targeting site, but both used the same HDR donor template. FIGS. 2B-2C show HDR efficiency at the different AAVS1 locus. FIG. 2D is a schematic of an insertion strategy at the human ACTB locus. FIGS. 2E-2F illustrate flow cytometry data showing that the HDR efficiency was significantly improved after activating CDK1 and repressing KU80 genes. Significance was calculated using the Unpaired t test. * P<0.05, ** P<0.01.

FIGS. 3A-3F illustrate an inducible DNA repair CRISPRa/i system for enhancing HDR efficiency. FIG. 3A is a diagram of TRE-MPH and TRE-CK expression vectors used to activate CDK1 and repress KU80, respectively. When rtTA interacts with doxycycline, the complex binds to the TRE3G promoter, which then initiates transcription of MCP-P65-HSF1 or COM-KRAB. FIG. 3B shows the workflow of establishing an inducible HDR increasing system. Activation of CDK1 and/or repression of KU80 can be achieved by simply controlling the availability of doxycycline. Dox, doxycycline; Puro, puromycin. FIGS. 3C-3E illustrate HEK293-TRE-MPH, HEK293-TRE-CK, and HEK293-TRE-MPH-CK cell lines obtained based on HEK293-Cas9 cell line by G418 selection. Several random clones were picked for each cell line. Although the transcriptional levels of CDK1 activation or KU80 repression can vary between clones, the clones with significant CDK1 activation and/or KU80 repression have increased HDR efficiency. The transcription level of CDK1 and KU80 were determined by RT-qPCR after 2 days' of doxycycline treatment. FIG. 3F shows quantitative analysis results of HDR efficiency. Data are shown as the mean±SD from three independent experiments. Significance was calculated using the Paired t test. * P<0.05, ** P<0.01, *** P<0.001, **** P<0.0001.

FIGS. 4A-4D illustrate packaging the DNA repair CRISPRa/i system into lentivirus for enhancement of HDR efficiency with viral delivery. FIG. 4A shows the CDK1 activation plasmid reconstructed into a lentivirus backbone. Hygro, Hygromycin. FIG. 4B shows HEK239FT cells were transduced with Cas9-Blast lentivirus to establish a Cas9 constitutively expressed cell-line. Then, the HEK239FT-Cas9 cell-line was transduced with dgCDK1-MS2:MPH lentivirus, followed by 2-3 days Hygromycin selection. Finally, cells were transfected with sgAAVS1-Puro plasmid and SA-T2A-EGFP HR donor. Flow cytometry analysis was performed after 2 days' puromycin selection. Blast, Blasticidin; Puro, Puromycin. FIG. 4C shows flow cytometry results demonstrating that HDR efficiency was significantly increased as compared with the vector group. FIG. 4D is a schematic diagram representing the central idea of the present study: with a single Cas9, through combinatorial usage of sgRNA and dgRNA for gene editing and CRISPRa/i on HDR/NHEJ machinery, HDR efficiency enhancement was achieved.

FIGS. 5A-5J illustrate functional tests of the dgRNA-Com:CK and dgRNA-MS2:MPH expression vectors. FIG. 5A is a schematic of plasmids used for testing dgRNA-Com:CK and dgRNA-MS2:MPH systems. FIG. 5B shows confocal analysis of dgRNA-Com:CK and dgRNA-MS2:MPH systems in HEK293 cells. HEK293 cells were transfected with pSV40-EGFP plasmid. One day later, the dgRNA-Com:CK or dgRNA-MS2:MPH expression vector targeting SV40 promoter (pSV40) was transfected. After 2 days, the fluorescence intensity was assessed using confocal microscopy. FIG. 5C shows quantitative fluorescence intensity of EGFP after activation and repression. FIGS. 5D-5E show the activation efficiency of ASLC1 (FIG. 5D) and HBG1 (FIG. 5E) in HEK293 cells using dgRNA-MS2:MPH expression vector targeting ASLC1 or HBG1 promoter regions. Three days later, total RNA was extracted and the gene transcriptional level was determined by RT-qPCR. FIGS. 5F-5J show the activation or suppression efficiency of essential genes related to DNA repair. Five dgRNAs were designed for each gene to screen the best dgRNA for CDK1 and CtIP activation and LIG4, KU80 and KU70 repression. Data were represented as the mean±SD from three independent experiments. Significance was calculated using the Paired t test. * P<0.05, ** P<0.01, *** P<0.001, **** P<0.0001.

FIGS. 6A-6C illustrate using the TLR reporter to evaluate HDR efficiency enhancement and confocal microscopy analysis. FIG. 6A shows the strategy used in this experiment. Firstly, cells were transfected with dgRNA-Com:CK or dgRNA-MS2:MPH vector to active or repress the targeted gene. After 1 day, these cells were co-transfected with EGFP HR donor and sgVenus vector. 2.5 days later, the samples were analyzed by confocal microscopy or flow cytometry. FIG. 6B shows HEK293-Cas9-TLR cells co-transfected with dgRNA-Com:CK or dgRNA-MS2:MPH plasmids and sgVenus vector. After 2 days, mCherry⁺ cells were analyzed by confocal microscopy. FIG. 6C shows HEK293-Cas9-TLR cells were co-transfected with intact EGFP PCR repair template and sgVenus plasmids after dgRNA-Com:CK or dgRNA-MS2:MPH plasmid transfection. 3 days later, samples were analyzed by confocal microscopy. The ratio of HDR-positive events was significantly increased after programming DNA repair pathways.

FIGS. 7A-7C illustrate NHEJ and HDR efficiency evaluation by the TLR system using FACS. FIG. 7A shows the AAVS1 sgRNA plasmid schematics (upper) and the workflow of this experiment (lower). FIG. 7B shows FACS gating settings for TRL analysis of HDR and NHEJ. FIG. 7C shows the HEK293-Cas9-TLR cell line was first transfected with dgRNA-MS2:MPH and/or dgRNA-Com:CK plasmids; 24 h later, these cells were co-transfected with intact EGFP PCR repair template and sgVenus-ECFP plasmid. FACS analysis was performed after 72 h of transfection, where ECFP⁺ cells were positively gated for transfection, and the percentage of Venus⁺ (HDR) cells and mCherry⁺ (NHEJ) cells were determined.

FIGS. 8A-8D illustrates sequencing confirmation of HDR- and NHEJ-positive events and exogenous gene into the endogenous AAVS1 locus. FIGS. 8A-8C show GFP⁺/mCherry⁻ (FIG. 8A), GFP⁻/mCherry⁺ (FIG. 8B) and GFP⁻/mCherry⁻ (FIG. 8C) individual clones were randomly picked, cultured, PCR and Sanger sequenced. Sequences from multiple clones are shown. FIG. 8D shows sequencing confirmation of EGFP⁺ cell clones to make sure SA-T2A-EGFP was precisely integrated into AAVS1 locus.

FIG. 9A-9B shows FACS plots for AAVS1 targeting HDR enhancement using inducible CRISPRa/i system. FIG. 9A shows HEK293-TRE-MPH, HEK293-TRE-CK, and HEK293-TRE-MPH-CK cell lines were co-transfected with SA-T2A-EGFP donor and sgAAVS1-mCherry plasmid, 24 h later, 1 μg/ml doxycycline was provided. After 2 days' doxycycline treatment, the frequency of EGFP⁺ cells within the population of mCherry⁺ cells were analyzed by flow cytometry. FIG. 9B shows cell viability detected after Doxycycline treatment.

FIGS. 10A-10C illustrate cell viability and cell cycle confirmation after programming HDR and NHEJ pathways using CRISPRa/i system. FIGS. 10A-10B show cell viability measured after doxycycline treatment. FIG. 10C shows cell cycle detected by Flow Cytometry after programming HDR and NHEJ pathways.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. “About” as used herein when referring to a measurable value such as an amount, a temporal duration, and the like, is meant to encompass variations of ±20% or ±10%, more preferably ±5%, even more preferably ±1%, and still more preferably ±0.1% from the

As used herein, the term “autologous” is meant to refer to any material derived from the same individual to which it is later to be re-introduced into the individual.

“Allogeneic” refers to any material derived from a different animal of the same species.

As used herein, the term “bp” refers to base pair.

The term “complementary” refers to the degree of anti-parallel alignment between two nucleic acid strands. Complete complementarity requires that each nucleotide be across from its opposite. No complementarity requires that each nucleotide is not across from its opposite. The degree of complementarity determines the stability of the sequences to be together or anneal/hybridize. Furthermore various DNA repair functions as well as regulatory functions are based on base pair complementarity.

The term “CRISPR/Cas” or “clustered regularly interspaced short palindromic repeats” or “CRISPR” refers to DNA loci containing short repetitions of base sequences followed by short segments of spacer DNA from previous exposures to a virus or plasmid. Bacteria and archaea have evolved adaptive immune defenses termed CRISPR/CRISPR-associated (Cas) systems that use short RNA to direct degradation of foreign nucleic acids. In bacteria, the CRISPR system provides acquired immunity against invading foreign DNA via RNA-guided DNA cleavage.

The “CRISPR/Cas9” system or “CRISPR/Cas9-mediated gene editing” refers to a type II CRISPR/Cas system that has been modified for genome editing/engineering. It is typically comprised of a “guide” RNA (gRNA) and a non-specific CRISPR-associated endonuclease (Cas9). “Guide RNA (gRNA)” is used interchangeably herein with “short guide RNA (sgRNA)” or “single guide RNA (sgRNA). The sgRNA is a short synthetic RNA composed of a “scaffold” sequence necessary for Cas9-binding and a user-defined ˜20 nucleotide “spacer” or “targeting” sequence which defines the genomic target to be modified. The genomic target of Cas9 can be changed by changing the targeting sequence present in the sgRNA.

“CRISPRa” system refers to a modification of the CRISPR-Cas9 system that functions to activate or increase gene expression. In certain embodiments, the CRISPRa system is comprised of dCas9, at least one transcriptional activator, and at least one sgRNA that functions to increase expression of at least one gene of interest.

“dCas9” as used herein refers to a catalytically dead Cas9 protein that lacks endonuclease activity.

“dgRNA” or “dead guide RNA” refers to a guide RNA which is catalytically inactive yet maintains target-site binding capacity.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockout” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient vectors for gene delivery. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

A “mutation” as used herein is a change in a DNA sequence resulting in an alteration from a given reference sequence (which may be, for example, an earlier collected DNA sample from the same subject). The mutation can comprise deletion and/or insertion and/or duplication and/or substitution of at least one deoxyribonucleic acid base such as a purine (adenine and/or thymine) and/or a pyrimidine (guanine and/or cytosine). Mutations may or may not produce discernible changes in the observable characteristics (phenotype) of an organism (subject).

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T”.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Parenteral” administration of an immunogenic composition includes, e.g., subcutaneous (s.c.), intravenous (i.v.), intramuscular (i.m.), or intrasternal injection, or infusion techniques.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

A “sample” or “biological sample” as used herein means a biological material from a subject, including but is not limited to organ, tissue, exosome, blood, plasma, saliva, urine and other body fluid. A sample can be any source of material obtained from a subject.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

CRISPR systems have been proven as versatile tools for site-specific genome engineering in mammalian species. During the gene editing processes, these RNA-guide nucleases introduce DNA double strand breaks (DSBs), in which non-homologous end joining (NHEJ) dominates the DNA repair pathway, limiting the efficiency of homology-directed repair (HDR), the alternative pathway essential for precise gene targeting. Multiple approaches have been developed to enhance HDR, including chemical compound or RNA interference mediated inhibition of NHEJ factors, small molecule activation of HDR enzymes, or cell cycle timed delivery of CRISPR complex. However, these approaches face multiple challenges, yet have moderate or variable effects. Herein, a new approach was developed that programs both NHEJ and HDR pathways with CRISPR activation and interference (CRISPRa/i) to achieve significantly enhanced HDR efficiency of CRISPR mediated gene editing. The manipulation of NHEJ and HDR pathway components, such as CtIP, CDK1, KU70, KU80 and LIG4, was performed with dead guide RNAs (dgRNAs), thus relying on only a single catalytically active Cas9 to perform CRISPRa/i as well as precise gene editing. While reprogramming of most DNA repair factors or their combinations tested enhanced HDR efficiency, simultaneously activating CDK1 and repressing KU80 has strongest effect with nearly 4-8-fold improvement. Doxycycline-induced dgRNA-based CRISPRa/i programming of DNA repair enzymes as well as viral packaging enabled flexible and tunable HDR enhancement in mammalian cells. This study provides an effective, flexible and safer strategy to enhance precise genome modifications, which broadly impacts human gene editing and therapy.

As described herein, the compositions and methods described herein provide many advantages including but not limited to: 1) the manipulation of NHEJ and HDR pathway components, such as CtIP, CDK1, KU70, KU80 and LIG4, was performed with a dead guide RNA (dgRNA), thus relying on only a single catalytically active Cas9 to perform CRISPRa/i as well as precise gene editing. 2) Reprogramming of most DNA repair factors or combinations tested enhanced HDR efficiency. 3) With simultaneously activation of CDK1 by dgRNA-MS2:MPH and/or repression of KU80 by dgRNA-Com:CK, the HDR efficiency can be enhanced by over an order of magnitude (upto 13 fold enhancement in two independent cell lines, one of the strongest effect among all methods available). 4) This is a genetic approach; thus the components can join force with an armamentarium of other genetic tools such as inducible gene expression modules via simple genetic engineering. 5) The CRISPRa/i constructs can be packaged into viral vectors for efficient delivery into a large repertoire of cell types. 6) Finally, this approach of HDR enhancement thus can be easily adapted for in vivo settings, which is essential for the application of gene therapy.

### Compositions

Certain aspects of the invention include compositions comprising plasmids, vectors, and kits for use in enhancing homology directed repair (HDR) and/or reducing non-homologous end joining (NHEJ) in a cell following CRISPR-mediated editing.

In certain embodiments, the invention includes use of “dead guide RNAs” (dgRNAs). Recently, these 14-nt or 15-nt guide RNAs have been shown to be catalytically inactive yet maintain target-site binding capacity (Kiani et al. (2015) Nat Methods 12, 1051-1054; Dahlman et al. (2015) Nat Biotechnol 33(11): 1159-1161). Thus, these catalytically dead guide RNAs (dgRNAs) can be utilized to modulate gene expression using a catalytically active Cas9. Therefore, an active Cas9 nuclease can be repurposed to simultaneously perform genome editing and regulate gene transcription using both types of gRNAs in the same cell. As demonstrated herein, dgRNAs together with the associated CRISPR activation (CRISPRa) and interference (CRISPRi) modules are deployed to achieve HDR enhancement using a single active Cas9.

In one aspect, the invention provides an activation plasmid/vector (dgRNA-MS2:MPH). The vector utilizes the MS2-P65-HSF (MPH) activation complex, which mediates efficient target upregulation by binding to MS2 loops in the dgRNA (Konermann et al. (2013) Nature 500:472-476). In one embodiment, the vector comprises a first promoter, a dead guide RNA (dgRNA) comprising a 14-15 base pair (bp) sequence that targets a homology directed repair (HDR) gene and two MS2 binding loops, a second promoter, a MS2 bacteriophage coat protein (MCP) sequence, and a P65-HSF1 sequence. In one embodiment, the vector comprises SEQ ID NO: 1. The HDR gene can include but is not limited to CDK1, CtIP, BRCA1/2, RAD50, and RAD51. In one embodiment, the sequence that targets a HDR gene is selected from the group consisting of SEQ ID NOs: 3-12.

In another aspect, the invention includes a repression plasmid/vector (dgRNA-Com:CK). The vector utilizes a Com-KRAB (CK) fusion domain. KRAB is a potent transcriptional repressor that recruits chromatin modifiers to silence target genes (Groner et al (2010) PLos Genet. 6:e1000869). Com is a well-characterized viral RNA sequence recognized by Com RNA binding protein (Zalatan et al. (2015) Cell 160(0):339-350). In certain embodiments of the vectors presented herein, a Com binding loop was constructed into a dgRNA scaffold for recruiting the Com-KRAB (CK) fusion domain to repress NHEJ-related genes. In one embodiment, the vector comprises a first promoter, a dgRNA comprising a 14-15 base pair (bp) sequence that targets a non-homologous end joining (NHEJ) gene and a Com binding loop, a second promoter, a Com sequence, and KRAB sequence. In one embodiment, the vector comprises SEQ ID NO: 2. Examples of NHEJ genes include but are not limited to LIG4, KU70 and KU80. In one embodiment, the NHEJ sequence is selected from the group consisting of SEQ ID NOs. 13-22.

In yet another aspect, the invention includes inducible repression and activation plasmids/vectors. In one embodiment, the vector comprises a first promoter sequence, an rtTA sequence, a second promoter sequence, a dead guide RNA (dgRNA) comprising a 14-15 base pair (bp) sequence that targets a HDR gene and two MS2 binding loops, a TREG3G promoter sequence, an MCP sequence, and a P65-HSF1 sequence. In one embodiment, the vector comprises SEQ ID NO: 29. In one embodiment, the sequence that targets a HDR gene is selected from the group consisting of SEQ ID NOs: 3-12. In another embodiment, the vector comprises a first promoter sequence, an rtTA sequence, a second promoter, a dgRNA comprising a 14-15 base pair (bp) sequence that targets a NHEJ gene and a COM binding loop, a TREG3G promoter sequence, a COM sequence, and KRAB sequence. In one embodiment, the vector comprises SEQ ID NO: 30. In one embodiment, the NHEJ sequence is selected from the group consisting of SEQ ID NOs. 13-22.

Another aspect of the invention includes a traffic light reporter plasmid/vector. In one embodiment, the vector comprises a promoter, a nonfunctional green fluorescent reporter containing a CRISPR targeting site, a self cleaving peptide, and a red fluorescent reporter containing a 2-bp shifted reading frame. In certain embodiments, the nonfunctional green fluorescent reporter comprises an EGFP variant wherein codons 53-63 are disrupted. In one embodiment, the vector comprises the nucleotide sequence of SEQ ID NO: 31. In one embodiment, the vector comprises the nucleotide sequence of SEQ ID NO: 32.

Any promoter known to one of ordinary skill in the art can be incorporated into any of the vectors/plasmids of the present invention. Suitable promoter and enhancer elements are known to those of skill in the art. For expression in a bacterial cell, suitable promoters include, but are not limited to, lad, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In one embodiment, the vector comprises a CMV promoter and/or a U6 promoter. Certain embodiments of the invention include more than one promoter per plasmid/vector. It should be known to one of ordinary skill in the art that the when a plasmid/vector comprises more than one promoter, said promoters can include two or more of the same promoter or two or more different promoters. For example, the vector may comprise a first promoter comprising a CMV promoter and a second promoter comprising a U6 promoter.

In addition, any of the vectors/plasmids of the present invention can include additional components. For example, the vector can further comprise an NLS sequence, a linker sequence, a polyA sequence, an SV40 sequence, and an antibiotic resistance gene/sequence. Any antibiotic resistance gene/sequence or selection marker known to one of ordinary skill in the art can be include in the vector. For example, the vector can comprise a Zeocin sequence. In one embodiment, the vector comprises a Hygromycin sequence.

The invention should be construed to encompass any type of vector known to one of ordinary skill in the art. For example, the vector can comprise a lentivirus, but can also comprise other viral vectors including but not limited to adenovirus, adeno-associated virus, retrovirus, hybrid viral vectors, or any combinations thereof. In one embodiment, the vector comprises a lentiviral backbone. In one embodiment, the vector comprises the nucleotide sequence of SEQ ID NO: 38.

In another aspect, the invention includes a cell or cell line comprising any of the plasmids/vectors of the present invention. Any type of cell line known to one of ordinary skill in the art can be utilized. For example, the invention can include a human embryonic kidney 293 (HEK293) cell or cell line comprising a plasmid/vector of the present invention. Other cell types include but are not limited to HeLa cells, T cells, autologous cells, and CAR T cells. The cell can include addition components, including but not limited to components useful for gene editing. For example, Cas9 can be included in the cell. Cas9 can be administered to the cell in any form, such as a plasmid, DNA, RNA, and protein.

### Methods

Certain aspects of the invention include methods for increasing homology directed repair (HDR) and/or decreasing non-homolgous end joining (NHEJ) in a cell. Certain aspects include methods for gene editing in a cell or in an animal.

One aspect of the invention includes a method of enhancing homology directed repair (HDR) and/or decreasing DNA non-homologous end-joining (NHEJ) following CRISPR editing in a cell. The method comprises administering to the cell a Cas9, a sgRNA, an activation plasmid, and a HDR donor template. The activation plasmid comprises a first promoter, a dead guide RNA (dgRNA) comprising a 14-15 base pair (bp) sequence that targets a homology directed repair (HDR) gene and two MS2 binding loops, a second promoter, an MCP sequence, and a P65-HSF1 sequence.

Another aspect of the invention includes a method of enhancing homology directed repair (HDR) and/or decreasing DNA non-homologous end-joining (NHEJ) following CRISPR editing in a cell comprising administering to the cell a Cas9, a sgRNA, a repression plasmid, and a HDR donor template. The repression plasmid comprises a first promoter, a dgRNA comprising a 14-15 base pair (bp) sequence that targets a non-homologous end joining (NHEJ) gene and a Com binding loop, a second promoter, a Com sequence, and KRAB sequence.

Yet another aspect of the invention includes a method of enhancing homology directed repair (HDR) and/or decreasing DNA non-homologous end-joining (NHEJ) following CRISPR editing in a cell, comprising administering to the cell a Cas9, a sgRNA, an activation plasmid, a repression plasmid, and a HDR donor template. The activation plasmid comprises a first promoter, a dead guide RNA (dgRNA) comprising a 14-15 base pair (bp) sequence that targets a homology directed repair (HDR) gene and two MS2 binding loops, a second promoter, an MCP sequence, and a P65-HSF1 sequence. The repression plasmid comprises a first promoter, a dgRNA comprising a 14-15 base pair (bp) sequence that targets a non-homologous end joining (NHEJ) gene and a Com binding loop, a second promoter, a Com sequence, and KRAB sequence.

In one embodiment, the activation plasmid targets CDK1-2 and/or the repression plasmid targets KU80-1. In one embodiment, the HDR gene is selected from the group consisting of CDK1, CtIP, BRCA1/2, RAD50, and RAD51. In one embodiment, NHEJ gene is selected from the group consisting of LIG4, KU70 and KU80. In one embodiment, the sequence that targets a HDR gene is selected from the group consisting of SEQ ID NOs: 3-12. In one embodiment, the sequence that targets a NHEJ gene is selected from the group consisting of SEQ ID NOs. 13-22.

In one embodiment, the activation plasmid comprises SEQ ID NO: 1. In one embodiment, the repression plasmid comprises SEQ ID NO: 2.

The repression and/or activation plasmid can be designed to further comprise an inducible expression system. For example, a Tet-On system can be included in the plasmid, which is inducible by doxycycline (Dox).

The first promoter of the repression and/or activation plasmid can comprise a CMV promoter or a U6 promoter and the second promoter of the repression and/or activation plasmid can comprise a CMV promoter or a U6 promoter. The repression and/or activation plasmid may further comprise additional components including but not limited to a NLS sequence, a linker sequence, a polyA sequence, an SV40 sequence, and an antibiotic resistance sequence.

The sgRNAs can be designed to target any gene or non-coding region of interest.

The repression and/or activation plasmids can be packaged into a lentiviral vector and be administered to an animal. In one embodiment, the animal is a human. Administration to the animal may be performed by any means known to one of ordinary skill in the art.

### CRISPR/Cas9

The CRISPR/Cas9 system is a facile and efficient system for inducing targeted genetic alterations. Target recognition by the Cas9 protein requires a ‘seed’ sequence within the guide RNA (gRNA) and a conserved dinucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 2931 cells), primary cells, and CAR T cells. The CRISPR/Cas9 system can simultaneously target multiple genomic loci by co-expressing a single Cas9 protein with two or more gRNAs, making this system uniquely suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, FINK and RuvC. The Reel domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US20140068797. CRISPRi induces permanent gene disruption that utilizes the RNA-guided Cas9 endonuclease to introduce DNA double stranded breaks, which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleotide sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, an pAd5F35-CRISPR vector. In other embodiments, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combinations thereof.

In certain embodiments, inducing the Cas9 expression vector comprises exposing the cell to an agent that activates an inducible promoter in the Cas9 expression vector. In such embodiments, the Cas9 expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

In certain embodiments, guide RNA(s) and Cas9 can be delivered to a cell as a ribonucleoprotein (RNP) complex. RNPs are comprised of purified Cas9 protein complexed with gRNA and are well known in the art to be efficiently delivered to multiple types of cells, including but not limited to stem cells and immune cells (Addgene, Cambridge, Mass., Mirus Bio LLC, Madison, Wis.).

The guide RNA is specific for a genomic region of interest and targets that region for Cas endonuclease-induced double strand breaks. The target sequence of the guide RNA sequence may be within a loci of a gene or within a non-coding region of the genome. In certain embodiments, the guide nucleotide sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

Guide RNA (gRNA), also referred to as “short guide RNA” or “sgRNA”, provides both targeting specificity and scaffolding/binding ability for the Cas9 nuclease. The gRNA can be a synthetic RNA composed of a targeting sequence and scaffold sequence derived from endogenous bacterial crRNA and tracrRNA. gRNA is used to target Cas9 to a specific genomic locus in genome engineering experiments. Guide RNAs can be designed using standard tools well known in the art.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as a DNA or a RNA polynucleotide. In certain embodiments, a target sequence is located in the nucleus or cytoplasm of a cell. In other embodiments, the target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, which is incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In certain embodiments, the CRISPR/Cas is derived from a type II CRISPR/Cas system. In some embodiments, the CRISPR/Cas sytem is derived from a Cas9 protein. The Cas9 protein can be from Streptococcus pyogenes, Streptococcus thermophilus, or other species.

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the present invention. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present invention may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4^{th} Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

### Introduction of Nucleic Acids

In certain embodiments an expression system is used for the introduction of gRNAs and (d)Cas9 proteins into the cells of interest. Typically employed options include but are not limited to plasmids and viral vectors such as adeno-associated virus (AAV) vector or lentivirus vector.

Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Moreover, the nucleic acids may be introduced by any means, such as transducing the cells, transfecting the cells, and electroporating the cells. One nucleic acid may be introduced by one method and another nucleic acid may be introduced into the cell by a different method.

RNA

In one embodiment, the nucleic acids introduced into the cell are RNA. In another embodiment, the RNA is mRNA that comprises in vitro transcribed RNA or synthetic RNA. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA.

PCR can be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary”, as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has a cap on the 5′ end and a 3′ poly(A) tail which determine ribosome binding, initiation of translation and stability mRNA in the cell. On a circular DNA template, for instance, plasmid DNA, RNA polymerase produces a long concatameric product which may not be suitable for expression in eukaryotic cells. The transcription of plasmid DNA linearized at the end of the 3′ UTR results in normal sized mRNA which may not be effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003)).

The conventional method of integration of polyA/T stretches into a DNA template is by molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

In some embodiments, the RNA is electroporated into the cells, such as in vitro transcribed RNA.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

One advantage of RNA transfection methods of the invention is that RNA transfection is essentially transient and vector-free. A RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

RNA has several advantages over more traditional plasmid or viral approaches. Gene expression from an RNA source does not require transcription and the protein product is produced rapidly after the transfection. Further, since the RNA has to only gain access to the cytoplasm, rather than the nucleus, and therefore typical transfection methods result in an extremely high rate of transfection. In addition, plasmid based approaches require that the promoter driving the expression of the gene of interest be active in the cells under study.

In another aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

### Sources of Cells

In one embodiment, cells are obtained from a subject. Non-limiting examples of subjects include humans, dogs, cats, mice, rats, pigs and transgenic species thereof. Preferably, the subject is a human. Cells can be obtained from a number of sources, including peripheral blood mononuclear cells, bone marrow, lymph node tissue, spleen tissue, umbilical cord, cancer cells and tumors. In certain embodiments, any number of cell lines available in the art, may be used. In certain embodiments, cells can be obtained from a unit of blood collected from a subject using any number of techniques known to the skilled artisan, such as Ficoll separation. In one embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, cells are isolated from peripheral blood. Alternatively, cells can be isolated from umbilical cord. In any event, a specific subpopulation of cells can be further isolated by positive or negative selection techniques.

Cells can also be frozen. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

### Pharmaceutical Compositions

Pharmaceutical compositions of the present invention may comprise the modified cell as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

It can generally be stated that a pharmaceutical composition comprising the modified cells described herein may be administered at a dosage of 10⁴ to 10⁹ cells/kg body weight, in some instances 10⁵ to 10⁶ cells/kg body weight, including all integer values within those ranges. Compositions of the invention may also be administered multiple times at these dosages. The cells or vectors can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

The administration of the modified cells or vectors of the invention may be carried out in any convenient manner known to those of skill in the art. The cells or vectors of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullarly, intracystically intramuscularly, by intravenous (i.v.) injection, parenterally or intraperitoneally. In other instances, the cells of the invention are injected directly into a site of inflammation in the subject, a local disease site in the subject, a lymph node, an organ, a tumor, and the like.

It should be understood that the methods and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### Experimental Examples

The invention is now described with reference to the following Examples. These Examples are provided for the purpose of illustration only, and the invention is not limited to these Examples, but rather encompasses all variations that are evident as a result of the teachings provided herein.

The materials and methods employed in these experiments are now described.

Generation of activation and repression plasmids: The activation plasmid dgRNA-MS2:MPH comprises a U6 promoter, an MS2 gRNA scaffold, a CMV promoter and a MCP-P65-HSF1 complex (SEQ ID NO:1). The repression plasmid dgRNA-Com:CK comprises a U6 promoter, a Com gRNA scaffold, a CMV promoter and a COM-KRAB complex (SEQ ID NO:2). All key DNA fragments in these plasmids were synthesized by GENEWIZ or IDT, then cloned into pUC57, or lentiviral plasmids using general molecular cloning and Gibson assembly (NEB). dgRNAs (14-nt or 15-nt) were designed to target the first 200 bp upstream of each TSS (Table 1, SEQ ID NOs. 3-28). Five dgRNAs were designed to target each gene. TRE-MPH (SEQ ID NO: 29) and TRE-CK (SEQ ID NO: 30) were constructed based on dgRNA-MS2:MPH and dgRNA-Com:CK by inserting CMV-rtTA cassette and replacing the CMV promoter, which drives MPH or CK expression, with a TRE3G inducible promoter. For establishment of TRE-MPH, TRE-CK, and TRE-MPH-CK cell lines, HEK293 cells were transduced with Cas9-expressing lentivirus to establish a constitutive Cas9 expression cell line, then transfected with TRE-MPH and/or TRE-CK plasmids followed by G418 selection and PCR identification.

```
Activation plasmid dgRNA-MS2:MPH:
(SEQ ID NO: 1)
      1 tcgcgcgttt cggtgatgac ggtgaaaacc tctgacacat gcagctcccg gagacggtca
    61 cagcttgtct gtaagcggat gccgggagca gacaagcccg tcagggcgcg tcagcgggtg
  121 ttggcgggtg tcggggctgg cttaactatg cggcatcaga gcagattgta ctgagagtgc
  181 accatatgcg gtgtgaaata ccgcacagat gcgtaaggag aaaataccgc atcaggcgcc
  241 attcgccatt caggctgcgc aactgttggg aagggcgatc ggtgcgggcc tcttcgctat
  301 tacgccagct ggcgaaaggg ggatgtgctg caaggcgatt aagttgggta acgccagggt
  361 tttcccagtc acgacgttgt aaaacgacgg ccagtgaatt cgagggccta tttcccatga
  421 ttccttcata tttgcatata cgatacaagg ctgttagaga gataattgga attaatttga
  481 ctgtaaacac aaagatatta gtacaaaata cgtgacgtag aaagtaataa tttcttgggt
  541 agtttgcagt tttaaaatta tgttttaaaa tggactatca tatgcttacc gtaacttgaa
  601 agtatttcga tttcttggct ttatatatct tgtggaaagg acgaaacacc gggtcttcga
  661 gaagacctgt tttagagcta ggccaacatg aggatcaccc atgtctgcag ggcctagcaa
  721 gttaaaataa ggctagtccg ttatcaactt ggccaacatg aggatcaccc atgtctgcag
  781 ggccaagtgg caccgagtcg gtgctttttg gtacccgtta cataacttac ggtaaatggc
  841 ccgcctggct gaccgcccaa cgacccccgc ccattgacgt caataatgac gtatgttccc
  901 atagtaacgc caatagggac tttccattga cgtcaatggg tggagtattt acggtaaact
  961 gcccacttgg cagtacatca agtgtatcat atgccaagta cgccccctat tgacgtcaat
1021 gacggtaaat ggcccgcctg gcattatgcc cagtacatga ccttatggga ctttcctact
1081 tggcagtaca tctacgtatt agtcatcgct attaccatgg tgatgcggtt ttggcagtac
1141 atcaatgggc gtggatagcg gtttgactca cggggatttc caagtctcca ccccattgac
1201 gtcaatggga gtttgttttg gcaccaaaat caacgggact ttccaaaatg tcgtaacaac
1261 tccgccccat tgacgcaaat gggcggtagg cgtgtacggt gggaggtcta tataagcaga
1321 gcttagtcta gaatgcccaa aaagaaaaga aaagtgggta gtatggcttc aaactttact
1381 cagttcgtgc tcgtggacaa tggtgggaca ggggatgtga cagtggctcc ttctaatttc
1441 gctaatgggg tggcagagtg gatcagctcc aactcacgga gccaggccta caaggtgaca
1501 tgcagcgtca ggcagtctag tgcccagaag agaaagtata ccatcaaggt ggaggtcccc
1561 aaagtggcta cccagacagt gggcggagtc gaactgcctg tcgccgcttg gaggtcctac
1621 ctgaacatgg agctcactat cccaattttc gctaccaatt ctgactgtga actcatcgtg
1681 aaggcaatgc aggggctcct caaagacggt aatcctatcc cttccgccat cgccgctaac
1741 tcaggtatct acggaggagg tggaagcgga ggaggaggaa gcggaggagg aggtagcctc
1801 gagggaccta agaaaaagag gaaggtggcg gccgctggat ccccttcagg gcagatcagc
1861 aaccaggccc tggctctggc ccctagctcc gctccagtgc tggcccagac tatggtgccc
1921 tctagtgcta tggtgcctct ggcccagcca cctgctccag cccctgtgct gaccccagga
1981 ccaccccagt cactgagcgc tccagtgccc aagtctacac aggccggcga ggggactctg
2041 agtgaagctc tgctgcacct gcagttcgac gctgatgagg acctgggagc tctgctgggg
2101 aacagcaccg atcccggagt gttcacagat ctggcctccg tggacaactc tgagtttcag
2161 cagctgctga atcagggcgt gtccatgtct catagtacag ccgaaccaat gctgatggag
2221 taccccgaag ccattacccg gctggtgacc ggcagccagc ggccccccga ccccgctcca
2281 actcccctgg gaaccagcgg cctgcctaat gggctgtccg gagatgagga cttctcaagc
2341 atcgctgata tggactttag tgccctgctg tcacagattt cctctagtgg gcagggagga
2401 ggtggaagcg gcttcagcgt ggacaccagt gccctgctgg acctgttcag cccctcggtg
2461 accgtgcccg acatgagcct gcctgacctt gacagcagcc tggccagtat ccaagagctc
2521 ctgtctcccc aggagccccc caggcctccc gaggcagaga acagcagccc ggattcaggg
2581 aagcagctgg tgcactacac agcgcagccg ctgttcctgc tggaccccgg ctccgtggac
2641 accgggagca acgacctgcc ggtgctgttt gagctgggag agggctccta cttctccgaa
2701 ggggacggct tcgccgagga ccccaccatc tccctgctga caggctcgga gcctcccaaa
2761 gccaaggacc ccactgtctc ctgagggccc aacttgttta ttgcagctta taatggttac
2821 aaataaagca atagcatcac aaatttcaca aataaagcat ttttttcact gcattctagt
2881 tgtggtttgt ccaaactcat caatgtatct tagtcgacgt gtgtcagtta gggtgtggaa
2941 agtccccagg ctccccagca ggcagaagta tgcaaagcat gcatctcaat tagtcagcaa
3001 ccaggtgtgg aaagtcccca ggctccccag caggcagaag tatgcaaagc atgcatctca
3061 attagtcagc aaccatagtc ccgcccctaa ctccgcccat cccgccccta actccgccca
3121 gttccgccca ttctccgccc catggctgac taattttttt tatttatgca gaggccgagg
3181 ccgcctctgc ctctgagcta ttccagaagt agtgaggagg cifitttgga ggcctaggct
3241 tttgcaaaaa gctcccggga gcttgtatat ccattttcgg atctgatcag cacgtgttga
3301 caattaatca tcggcatagt atatcggcat agtataatac gacaaggtga ggaactaaac
3361 catggccaag ttgaccagtg ccgttccggt gctcaccgcg cgcgacgtcg ccggagcggt
3421 cgagttctgg accgaccggc tcgggttctc ccgggacttc gtggaggacg acttcgccgg
3481 tgtggtccgg gacgacgtga ccctgttcat cagcgcggtc caggaccagg tggtgccgga
3541 caacaccctg gcctgggtgt gggtgcgcgg cctggacgag ctgtacgccg agtggtcgga
3601 ggtcgtgtcc acgaacttcc gggacgcctc cgggccggcc atgaccgaga tcggcgagca
3661 gccgtggggg cgggagttcg ccctgcgcga cccggccggc aactgcgtgc acttcgtggc
3721 cgaggagcag gactgacacg tgctacgaga tttcgattcc accgccgcct tctatgaaag
3781 gttgggcttc ggaatcgttt tccgggacgc cggctggatg atcctccagc gcggggatct
3841 catgctggag ttcttcgccc accccaactt gtttattgca gcttataatg gttacaaata
3901 aagcaatagc atcacaaatt tcacaaataa agcatttttt tcactgcatt ctagttgtgg
3961 tttgtccaaa ctcatcaatg tatcttaaag cttggcgtaa tcatggtcat agctgtttcc
4021 tgtgtgaaat tgttatccgc tcacaattcc acacaacata cgagccggaa gcataaagtg
4081 taaagcctgg ggtgcctaat gagtgagcta actcacatta attgcgttgc gctcactgcc
4141 cgctttccag tcgggaaacc tgtcgtgcca gctgcattaa tgaatcggcc aacgcgcggg
4201 gagaggcggt ttgcgtattg ggcgctcttc cgcttcctcg ctcactgact cgctgcgctc
4261 ggtcgttcgg ctgcggcgag cggtatcagc tcactcaaag gcggtaatac ggttatccac
4321 agaatcaggg gataacgcag gaaagaacat gtgagcaaaa ggccagcaaa aggccaggaa
4381 ccgtaaaaag gccgcgttgc tggcgttttt ccataggctc cgcccccctg acgagcatca
4441 caaaaatcga cgctcaagtc agaggtggcg aaacccgaca ggactataaa gataccaggc
4501 gtttccccct ggaagctccc tcgtgcgctc tcctgttccg accctgccgc ttaccggata
4561 cctgtccgcc tttctccctt cgggaagcgt ggcgctttct catagctcac gctgtaggta
4621 tctcagttcg gtgtaggtcg ttcgctccaa gctgggctgt gtgcacgaac cccccgttca
4681 gcccgaccgc tgcgccttat ccggtaacta tcgtcttgag tccaacccgg taagacacga
4741 cttatcgcca ctggcagcag ccactggtaa caggattagc agagcgaggt atgtaggcgg
4801 tgctacagag ttcttgaagt ggtggcctaa ctacggctac actagaagaa cagtatttgg
4861 tatctgcgct ctgctgaagc cagttacctt cggaaaaaga gttggtagct cttgatccgg
4921 caaacaaacc accgctggta gcggtggttt ttttgtttgc aagcagcaga ttacgcgcag
4981 aaaaaaagga tctcaagaag atcctttgat cttttctacg gggtctgacg ctcagtggaa
5041 cgaaaactca cgttaaggga ttttggtcat gagattatca aaaaggatct tcacctagat
5101 ccttttaaat taaaaatgaa gttttaaatc aatctaaagt atatatgagt aaacttggtc
5161 tgacagttac caatgcttaa tcagtgaggc acctatctca gcgatctgtc tatttcgttc
5221 atccatagtt gcctgactcc ccgtcgtgta gataactacg atacgggagg gcttaccatc
5281 tggccccagt gctgcaatga taccgcgaga cccacgctca ccggctccag atttatcagc
5341 aataaaccag ccagccggaa gggccgagcg cagaagtggt cctgcaactt tatccgcctc
5401 catccagtct attaattgtt gccgggaagc tagagtaagt agttcgccag ttaatagttt
5461 gcgcaacgtt gttgccattg ctacaggcat cgtggtgtca cgctcgtcgt ttggtatggc
5521 ttcattcagc tccggttccc aacgatcaag gcgagttaca tgatccccca tgttgtgcaa
5581 aaaagcggtt agctccttcg gtcctccgat cgttgtcaga agtaagttgg ccgcagtgtt
5641 atcactcatg gttatggcag cactgcataa ttctcttact gtcatgccat ccgtaagatg
5701 cttttctgtg actggtgagt actcaaccaa gtcattctga gaatagtgta tgcggcgacc
5761 gagttgctct tgcccggcgt caatacggga taataccgcg ccacatagca gaactttaaa
5821 agtgctcatc attggaaaac gttcttcggg gcgaaaactc tcaaggatct taccgctgtt
5881 gagatccagt tcgatgtaac ccactcgtgc acccaactga tcttcagcat cttttacttt
5941 caccagcgtt tctgggtgag caaaaacagg aaggcaaaat gccgcaaaaa agggaataag
6001 ggcgacacgg aaatgttgaa tactcatact cttccttttt caatattatt gaagcattta
6061 tcagggttat tgtctcatga gcggatacat atttgaatgt atttagaaaa ataaacaaat
6121 aggggttccg cgcacatttc cccgaaaagt gccacctgac gtctaagaaa ccattattat
6181 catgacatta acctataaaa ataggcgtat cacgaggccc tttcgtc
Repression plasmid dgRNA-Com:CK:
(SEQ ID NO: 2)
      1 tcgcgcgttt cggtgatgac ggtgaaaacc tctgacacat gcagctcccg gagacggtca
    61 cagcttgtct gtaagcggat gccgggagca gacaagcccg tcagggcgcg tcagcgggtg
  121 ttggcgggtg tcggggctgg cttaactatg cggcatcaga gcagattgta ctgagagtgc
  181 accatatgcg gtgtgaaata ccgcacagat gcgtaaggag aaaataccgc atcaggcgcc
  241 attcgccatt caggctgcgc aactgttggg aagggcgatc ggtgcgggcc tcttcgctat
  301 tacgccagct ggcgaaaggg ggatgtgctg caaggcgatt aagttgggta acgccagggt
  361 tttcccagtc acgacgttgt aaaacgacgg ccagtgaatt cgagggccta tttcccatga
  421 ttccttcata tttgcatata cgatacaagg ctgttagaga gataattgga attaatttga
  481 ctgtaaacac aaagatatta gtacaaaata cgtgacgtag aaagtaataa tttcttgggt
  541 agtttgcagt tttaaaatta tgttttaaaa tggactatca tatgcttacc gtaacttgaa
  601 agtatttcga tttcttggct ttatatatct tgtggaaagg acgaaacacc gggtcttcga
  661 gaagacctgt ttaagagcta tgctggaaac agcatagcaa gtttaaataa ggctagtccg
  721 ttatcaactt gaaaaagtgg caccgagtcg gtgcctgaat gcctgcgagc atcttttttt
  781 gttttttatg tctggtaccc gttacataac ttacggtaaa tggcccgcct ggctgaccgc
  841 ccaacgaccc ccgcccattg acgtcaataa tgacgtatgt tcccatagta acgccaatag
  901 ggactttcca ttgacgtcaa tgggtggagt atttacggta aactgcccac ttggcagtac
  961 atcaagtgta tcatatgcca agtacgcccc ctattgacgt caatgacggt aaatggcccg
1021 cctggcatta tgcccagtac atgaccttat gggactttcc tacttggcag tacatctacg
1081 tattagtcat cgctattacc atggtgatgc ggttttggca gtacatcaat gggcgtggat
1141 agcggtttga ctcacgggga tttccaagtc tccaccccat tgacgtcaat gggagtttgt
1201 tttggcacca aaatcaacgg gactttccaa aatgtcgtaa caactccgcc ccattgacgc
1261 aaatgggcgg taggcgtgta cggtgggagg tctatataag cagagcttag tctagaatgc
1321 ccaaaaagaa aagaaaagtg ggtagtatga aatcaattcg ctgtaaaaac tgcaacaaac
1381 tgttatttaa ggcggatagt tttgatcaca ttgaaatcag gtgtccgcgt tgcaaacgtc
1441 acatcataat gctgaatgcc tgcgagcatc ccacggagaa acattgtggg aaaagagaaa
1501 aaatcacgca ttctgacgaa accgtgcgtt atggaggagg tggaagcgga ggaggaggaa
1561 gcggaggagg aggtagcctc gagatggatg ctaagtcact aactgcctgg tcccggacac
1621 tggtgacctt caaggatgta tttgtggact tcaccaggga ggagtggaag ctgctggaca
1681 ctgctcagca gatcgtgtac agaaatgtga tgctggagaa ctataagaac ctggtttcct
1741 tgggttatca gcttactaag ccagatgtga tcctccggtt ggagaaggga gaagagccct
1801 aggggcccaa cttgtttatt gcagcttata atggttacaa ataaagcaat agcatcacaa
1861 atttcacaaa taaagcattt ttttcactgc attctagttg tggtttgtcc aaactcatca
1921 atgtatctta gtcgactgca gaggcctgca tgcaagcttg gcgtaatcat ggtcatagct
1981 gtttcctgtg tgaaattgtt atccgctcac aattccacac aacatacgag ccggaagcat
2041 aaagtgtaaa gcctggggtg cctaatgagt gagctaactc acattaattg cgttgcgctc
2101 actgcccgct ttccagtcgg gaaacctgtc gtgccagctg cattaatgaa tcggccaacg
2161 cgcggggaga ggcggtttgc gtattgggcg ctcttccgct tcctcgctca ctgactcgct
2221 gcgctcggtc gttcggctgc ggcgagcggt atcagctcac tcaaaggcgg taatacggtt
2281 atccacagaa tcaggggata acgcaggaaa gaacatgtga gcaaaaggcc agcaaaaggc
2341 caggaaccgt aaaaaggccg cgttgctggc gtttttccat aggctccgcc cccctgacga
2401 gcatcacaaa aatcgacgct caagtcagag gtggcgaaac ccgacaggac tataaagata
2461 ccaggcgttt ccccctggaa gctccctcgt gcgctctcct gttccgaccc tgccgcttac
2521 cggatacctg tccgcctttc tcccttcggg aagcgtggcg ctttctcata gctcacgctg
2581 taggtatctc agttcggtgt aggtcgttcg ctccaagctg ggctgtgtgc acgaaccccc
2641 cgttcagccc gaccgctgcg ccttatccgg taactatcgt cttgagtcca acccggtaag
2701 acacgactta tcgccactgg cagcagccac tggtaacagg attagcagag cgaggtatgt
2761 aggcggtgct acagagttct tgaagtggtg gcctaactac ggctacacta gaagaacagt
2821 atttggtatc tgcgctctgc tgaagccagt taccttcgga aaaagagttg gtagctcttg
2881 atccggcaaa caaaccaccg ctggtagcgg tggttttttt gtttgcaagc agcagattac
2941 gcgcagaaaa aaaggatctc aagaagatcc tttgatcttt tctacggggt ctgacgctca
3001 gtggaacgaa aactcacgtt aagggatttt ggtcatgaga ttatcaaaaa ggatcttcac
3061 ctagatcctt ttaaattaaa aatgaagttt taaatcaatc taaagtatat atgagtaaac
3121 ttggtctgac agttaccaat gcttaatcag tgaggcacct atctcagcga tctgtctatt
3181 tcgttcatcc atagttgcct gactccccgt cgtgtagata actacgatac gggagggctt
3241 accatctggc cccagtgctg caatgatacc gcgagaccca cgctcaccgg ctccagattt
3301 atcagcaata aaccagccag ccggaagggc cgagcgcaga agtggtcctg caactttatc
3361 cgcctccatc cagtctatta attgttgccg ggaagctaga gtaagtagtt cgccagttaa
3421 tagtttgcgc aacgttgttg ccattgctac aggcatcgtg gtgtcacgct cgtcgtttgg
3481 tatggcttca ttcagctccg gttcccaacg atcaaggcga gttacatgat cccccatgtt
3541 gtgcaaaaaa gcggttagct ccttcggtcc tccgatcgtt gtcagaagta agttggccgc
3601 agtgttatca ctcatggtta tggcagcact gcataattct cttactgtca tgccatccgt
3661 aagatgcttt tctgtgactg gtgagtactc aaccaagtca ttctgagaat agtgtatgcg
3721 gcgaccgagt tgctcttgcc cggcgtcaat acgggataat accgcgccac atagcagaac
3781 tttaaaagtg ctcatcattg gaaaacgttc ttcggggcga aaactctcaa ggatcttacc
3841 gctgttgaga tccagttcga tgtaacccac tcgtgcaccc aactgatctt cagcatcttt
3901 tactttcacc agcgtttctg ggtgagcaaa aacaggaagg caaaatgccg caaaaaaggg
3961 aataagggcg acacggaaat gttgaatact catactcttc ctttttcaat attattgaag
4021 catttatcag ggttattgtc tcatgagcgg atacatattt gaatgtattt agaaaaataa
4081 acaaataggg gttccgcgca catttccccg aaaagtgcca cctgacgtct aagaaaccat
4141 tattatcatg acattaacct ataaaaatag gcgtatcacg aggccctttc gtc
```

TABLE 1
Target sequences of dgRNAs

| Target gene | Name | Sequence (5′>3′) | SEQ ID NO: |
|---|---|---|---|
| CDK1 | dgCDK1-1 | GCGCTCTAGCCACC | SEQ ID NO: 3 |
| | dgCDK1-2 | ACGGGCTACCCGAT | SEQ ID NO: 4 |
| | dgCDK1-3 | GCGCTCGCACTCAGT | SEQ ID NO: 5 |
| | dgCDK1-4 | CTAGTCAGCGGAGC | SEQ ID NO: 6 |
| | dgCDK1-5 | GAACTGTGCCAATGC | SEQ ID NO: 7 |
| CtIP | dgCtIP-1 | GCGTGACGTCGCGC | SEQ ID NO: 8 |
| | dgCtIP-2 | GGGCAGCTGGAGGAA | SEQ ID NO: 9 |
| | dgCtIP-3 | ATCGCCCTCCGGGAT | SEQ ID NO: 10 |
| | dgCtIP-4 | GTCGCCAGACTCTTC | SEQ ID NO: 11 |
| | dgCtIP-5 | GCATCAAGCCCTTG | SEQ ID NO: 12 |
| Ligase IV | dgLIG4-1 | GGCCCTTAAAACTT | SEQ ID NO: 13 |
| | dgLIG4-2 | ACACTTCAGTGCAC | SEQ ID NO: 14 |
| | dgLIG4-3 | TACCTCGGCGGCGT | SEQ ID NO: 15 |
| | dgLIG4-4 | GAGCCCCCGCGACGG | SEQ ID NO: 16 |
| | dgLIG4-5 | GGGGCTCACTGGCAG | SEQ ID NO: 17 |
| KU70 | dgKU70-1 | GGTAGAAGCTGGTTG | SEQ ID NO: 18 |
| | dgKU70-2 | GTTGGCTTTCGTCA | SEQ ID NO: 19 |
| KU80 | dgKU80-1 | GCATGCTCAGAGTTC | SEQ ID NO: 20 |
| | dgKU80-2 | GCCTTTCAGGCCTAGC | SEQ ID NO: 21 |
| | dgKU80-3 | GTACTAGCGTTTCAGG | SEQ ID NO: 22 |
| ASCL1 | dgASCL1 | GCTCGCTGCAGCAG | SEQ ID NO: 23 |
| HBG1 | dgHBG1 | GAGGCCAGGGGCCGG | SEQ ID NO: 24 |
| EGFP | dgGFP-A1 | ATTAGTCAGCAACC | SEQ ID NO: 25 |
| EGFP | dgGFP-A2 | ACTGGGCGGAGTTAG | SEQ ID NO: 26 |
| EGFP | dgGFP-R1 | GGCCGAGGCCGCCT | SEQ ID NO: 27 |
| EGFP | dgGFP-R2 | CAGAAGTAGTGAGG | SEQ ID NO: 28 |

```
TRE-MPH
(SEQ ID NO: 29)
      1 gacggatcgg gagatctccc gatcccctat ggtgcactct cagtacaatc tgctctgatg
    61 ccgcatagtt aagccagtat ctgctccctg cttgtgtgtt ggaggtcgct gagtagtgcg
  121 cgagcaaaat ttaagctaca acaaggcaag gcttgaccga caattgcatg aagaatctgc
  181 ttagggttag gcgttttgcg ctgcttcgcg atgtacgggc cagatatacg cgttgacatt
  241 gattattgac tagttattaa tagtaatcaa ttacggggtc attagttcat agcccatata
  301 tggagttccg cgttacataa cttacggtaa atggcccgcc tggctgaccg cccaacgacc
  361 cccgcccatt gacgtcaata atgacgtatg ttcccatagt aacgccaata gggactttcc
  421 attgacgtca atgggtggag tatttacggt aaactgccca cttggcagta catcaagtgt
  481 atcatatgcc aagtacgccc cctattgacg tcaatgacgg taaatggccc gcctggcatt
  541 atgcccagta catgacctta tgggactttc ctacttggca gtacatctac gtattagtca
  601 tcgctattac catggtgatg cggttttggc agtacatcaa tgggcgtgga tagcggtttg
  661 actcacgggg atttccaagt ctccacccca ttgacgtcaa tgggagtttg ttttggcacc
  721 aaaatcaacg ggactttcca aaatgtcgta acaactccgc cccattgacg caaatgggcg
  781 gtaggcgtgt acggtgggag gtctatataa gcagagctct ctggctaact agagaaccca
  841 ctgcttactg gcttatcgaa attaatacga ctcactatag ggagacccaa gctggctagc
  901 atgtctagac tggacaagag caaagtcata aacggcgctc tggaattact caatggagtc
  961 ggtatcgaag gcctgacgac aaggaaactc gctcaaaagc tgggagttga gcagcctacc
1021 ctgtactggc acgtgaagaa caagcgggcc ctgctcgatg ccctgccaat cgagatgctg
1081 gacaggcatc atacccactt ctgccccctg gaaggcgagt catggcaaga ctttctgcgg
1141 aacaacgcca agtcattccg ctgtgctctc ctctcacatc gcgacggggc taaagtgcat
1201 ctcggcaccc gcccaacaga gaaacagtac gaaaccctgg aaaatcagct cgcgttcctg
1261 tgtcagcaag gcttctccct ggagaacgca ctgtacgctc tgtccgccgt gggccacttt
1321 acactgggct gcgtattgga ggaacaggag catcaagtag caaaagagga aagagagaca
1381 cctaccaccg attctatgcc cccacttctg agacaagcaa ttgagctgtt cgaccggcag
1441 ggagccgaac ctgccttcct tttcggcctg gaactaatca tatgtggcct ggagaaacag
1501 ctaaagtgcg aaagcggcgg gccggccgac gcccttgacg attttgactt agacatgctc
1561 ccagccgatg cccttgacga ctttgacctt gatatgctgc ctgctgacgc tcttgacgat
1621 tttgaccttg acatgctccc cgggtaaacc cagctttctt gtacaaagtg gtgatcttaa
1681 ggagggccta tttcccatga ttccttcata tttgcatata cgatacaagg ctgttagaga
1741 gataattgga attaatttga ctgtaaacac aaagatatta gtacaaaata cgtgacgtag
1801 aaagtaataa tttcttgggt agtttgcagt tttaaaatta tgttttaaaa tggactatca
1861 tatgcttacc gtaacttgaa agtatttcga tttcttggct ttatatatct tgtggaaagg
1921 acgaaacacc gggtcttcga gaagacctgt tttagagcta ggccaacatg aggatcaccc
1981 atgtctgcag ggcctagcaa gttaaaataa ggctagtccg ttatcaactt ggccaacatg
2041 aggatcaccc atgtctgcag ggccaagtgg caccgagtcg gtgettific ggatcccgat
2101 cacgagacta gcctcgagtt ggctttactc cctatcagtg atagagaacg tatgaagagt
2161 ttactcccta tcagtgatag agaacgtatg cagactttac tccctatcag tgatagagaa
2221 cgtataagga gtttactccc tatcagtgat agagaacgta tgaccagttt actccctatc
2281 agtgatagag aacgtatcta cagtttactc cctatcagtg atagagaacg tatatccagt
2341 ttactcccta tcagtgatag agaacgtata agctttaggc gtgtacggtg ggcgcctata
2401 aaagcagagc tcgtttagtg aaccgtcaga tcgcctggag caattccaca acacttttgt
2461 cttataccaa ctttccgtac cacttcctac cctcgtaaac cgcggccccg aattgcaagt
2521 ttgtacaaag gtaccatggc ttcaaacttt actcagttcg tgctcgtgga caatggtggg
2581 acaggggatg tgacagtggc tccttctaat ttcgctaatg gggtggcaga gtggatcagc
2641 tccaactcac ggagccaggc ctacaaggtg acatgcagcg tcaggcagtc tagtgcccag
2701 aagagaaagt ataccatcaa ggtggaggtc cccaaagtgg ctacccagac agtgggcgga
2761 gtcgaactgc ctgtcgccgc ttggaggtcc tacctgaaca tggagctcac tatcccaatt
2821 ttcgctacca attctgactg tgaactcatc gtgaaggcaa tgcaggggct cctcaaagac
2881 ggtaatccta tcccttccgc catcgccgct aactcaggta tctacggagg aggtggaagc
2941 ggaggaggag gaagcggagg aggaggtagc ctcgagggac ctaagaaaaa gaggaaggtg
3001 gcggccgctg gatccccttc agggcagatc agcaaccagg ccctggctct ggcccctagc
3061 tccgctccag tgctggccca gactatggtg ccctctagtg ctatggtgcc tctggcccag
3121 ccacctgctc cagcccctgt gctgacccca ggaccacccc agtcactgag cgctccagtg
3181 cccaagtcta cacaggccgg cgaggggact ctgagtgaag ctctgctgca cctgcagttc
3241 gacgctgatg aggacctggg agctctgctg gggaacagca ccgatcccgg agtgttcaca
3301 gatctggcct ccgtggacaa ctctgagttt cagcagctgc tgaatcaggg cgtgtccatg
3361 tctcatagta cagccgaacc aatgctgatg gagtaccccg aagccattac ccggctggtg
3421 accggcagcc agcggccccc cgaccccgct ccaactcccc tgggaaccag cggcctgcct
3481 aatgggctgt ccggagatga ggacttctca agcatcgctg atatggactt tagtgccctg
3541 ctgtcacaga tttcctctag tgggcaggga ggaggtggaa gcggcttcag cgtggacacc
3601 agtgccctgc tggacctgtt cagcccctcg gtgaccgtgc ccgacatgag cctgcctgac
3661 cttgacagca gcctggccag tatccaagag ctcctgtctc cccaggagcc ccccaggcct
3721 cccgaggcag agaacagcag cccggattca gggaagcagc tggtgcacta cacagcgcag
3781 ccgctgttcc tgctggaccc cggctccgtg gacaccggga gcaacgacct gccggtgctg
3841 tttgagctgg gagagggctc ctacttctcc gaaggggacg gcttcgccga ggaccccacc
3901 atctccctgc tgacaggctc ggagcctccc aaagccaagg accccactgt ctcctgagaa
3961 ttctgcagat atccagcaca gtggcggccg ctcgagtcta gagggcccgt ttaaacccgc
4021 tgatcagcct cgactgtgcc ttctagttgc cagccatctg ttgtttgccc ctcccccgtg
4081 ccttccttga ccctggaagg tgccactccc actgtccttt cctaataaaa tgaggaaatt
4141 gcatcgcatt gtctgagtag gtgtcattct attctggggg gtggggtggg gcaggacagc
4201 aagggggagg attgggaaga caatagcagg catgctgggg atgcggtggg ctctatggct
4261 tctgaggcgg aaagaaccag ctggggctct agggggtatc cccacgcgcc ctgtagcggc
4321 gcattaagcg cggcgggtgt ggtggttacg cgcagcgtga ccgctacact tgccagcgcc
4381 ctagcgcccg ctcctttcgc tttcttccct tcctttctcg ccacgttcgc cggctttccc
4441 cgtcaagctc taaatcgggg gctcccttta gggttccgat ttagtgcttt acggcacctc
4501 gaccccaaaa aacttgatta gggtgatggt tcacgtagtg ggccatcgcc ctgatagacg
4561 gtttttcgcc ctttgacgtt ggagtccacg ttctttaata gtggactctt gttccaaact
4621 ggaacaacac tcaaccctat ctcggtctat tcttttgatt tataagggat tttgccgatt
4681 tcggcctatt ggttaaaaaa tgagctgatt taacaaaaat ttaacgcgaa ttaattctgt
4741 ggaatgtgtg tcagttaggg tgtggaaagt ccccaggctc cccagcaggc agaagtatgc
4801 aaagcatgca tctcaattag tcagcaacca ggtgtggaaa gtccccaggc tccccagcag
4861 gcagaagtat gcaaagcatg catctcaatt agtcagcaac catagtcccg cccctaactc
4921 cgcccatccc gcccctaact ccgcccagtt ccgcccattc tccgccccat ggctgactaa
4981 ttttttttat ttatgcagag gccgaggccg cctctgcctc tgagctattc cagaagtagt
5041 gaggaggctt ttttggaggc ctaggctttt gcaaaaagct cccgggagct tgtatatcca
5101 ttttcggatc tgatcaagag acaggatgag gatcgtttcg catgattgaa caagatggat
5161 tgcacgcagg ttctccggcc gcttgggtgg agaggctatt cggctatgac tgggcacaac
5221 agacaatcgg ctgctctgat gccgccgtgt tccggctgtc agcgcagggg cgcccggttc
5281 tttttgtcaa gaccgacctg tccggtgccc tgaatgaact gcaggacgag gcagcgcggc
5341 tatcgtggct ggccacgacg ggcgttcctt gcgcagctgt gctcgacgtt gtcactgaag
5401 cgggaaggga ctggctgcta ttgggcgaag tgccggggca ggatctcctg tcatctcacc
5461 ttgctcctgc cgagaaagta tccatcatgg ctgatgcaat gcggcggctg catacgcttg
5521 atccggctac ctgcccattc gaccaccaag cgaaacatcg catcgagcga gcacgtactc
5581 ggatggaagc cggtcttgtc gatcaggatg atctggacga agagcatcag gggctcgcgc
5641 cagccgaact gttcgccagg ctcaaggcgc gcatgcccga cggcgaggat ctcgtcgtga
5701 cccatggcga tgcctgcttg ccgaatatca tggtggaaaa tggccgcttt tctggattca
5761 tcgactgtgg ccggctgggt gtggcggacc gctatcagga catagcgttg gctacccgtg
5821 atattgctga agagcttggc ggcgaatggg ctgaccgctt cctcgtgctt tacggtatcg
5881 ccgctcccga ttcgcagcgc atcgccttct atcgccttct tgacgagttc ttctgagcgg
5941 gactctgggg ttcgaaatga ccgaccaagc gacgcccaac ctgccatcac gagatttcga
6001 ttccaccgcc gccttctatg aaaggttggg cttcggaatc gttttccggg acgccggctg
6061 gatgatcctc cagcgcgggg atctcatgct ggagttcttc gcccacccca acttgtttat
6121 tgcagcttat aatggttaca aataaagcaa tagcatcaca aatttcacaa ataaagcatt
6181 tttttcactg cattctagtt gtggtttgtc caaactcatc aatgtatctt atcatgtctg
6241 tataccgtcg acctctagct agagcttggc gtaatcatgg tcatagctgt ttcctgtgtg
6301 aaattgttat ccgctcacaa ttccacacaa catacgagcc ggaagcataa agtgtaaagc
6361 ctggggtgcc taatgagtga gctaactcac attaattgcg ttgcgctcac tgcccgcttt
6421 ccagtcggga aacctgtcgt gccagctgca ttaatgaatc ggccaacgcg cggggagagg
6481 cggtttgcgt attgggcgct cttccgcttc ctcgctcact gactcgctgc gctcggtcgt
6541 tcggctgcgg cgagcggtat cagctcactc aaaggcggta atacggttat ccacagaatc
6601 aggggataac gcaggaaaga acatgtgagc aaaaggccag caaaaggcca ggaaccgtaa
6661 aaaggccgcg ttgctggcgt ttttccatag gctccgcccc cctgacgagc atcacaaaaa
6721 tcgacgctca agtcagaggt ggcgaaaccc gacaggacta taaagatacc aggcgtttcc
6781 ccctggaagc tccctcgtgc gctctcctgt tccgaccctg ccgcttaccg gatacctgtc
6841 cgcctttctc ccttcgggaa gcgtggcgct ttctcatagc tcacgctgta ggtatctcag
6901 ttcggtgtag gtcgttcgct ccaagctggg ctgtgtgcac gaaccccccg ttcagcccga
6961 ccgctgcgcc ttatccggta actatcgtct tgagtccaac ccggtaagac acgacttatc
7021 gccactggca gcagccactg gtaacaggat tagcagagcg aggtatgtag gcggtgctac
7081 agagttcttg aagtggtggc ctaactacgg ctacactaga agaacagtat ttggtatctg
7141 cgctctgctg aagccagtta ccttcggaaa aagagttggt agctcttgat ccggcaaaca
7201 aaccaccgct ggtagcggtt tttttgtttg caagcagcag attacgcgca gaaaaaaagg
7261 atctcaagaa gatcctttga tcttttctac ggggtctgac gctcagtgga acgaaaactc
7321 acgttaaggg attttggtca tgagattatc aaaaaggatc ttcacctaga tccttttaaa
7381 ttaaaaatga agttttaaat caatctaaag tatatatgag taaacttggt ctgacagtta
7441 ccaatgctta atcagtgagg cacctatctc agcgatctgt ctatttcgtt catccatagt
7501 tgcctgactc cccgtcgtgt agataactac gatacgggag ggcttaccat ctggccccag
7561 tgctgcaatg ataccgcgag acccacgctc accggctcca gatttatcag caataaacca
7621 gccagccgga agggccgagc gcagaagtgg tcctgcaact ttatccgcct ccatccagtc
7681 tattaattgt tgccgggaag ctagagtaag tagttcgcca gttaatagtt tgcgcaacgt
7741 tgttgccatt gctacaggca tcgtggtgtc acgctcgtcg tttggtatgg cttcattcag
7801 ctccggttcc caacgatcaa ggcgagttac atgatccccc atgttgtgca aaaaagcggt
7861 tagctccttc ggtcctccga tcgttgtcag aagtaagttg gccgcagtgt tatcactcat
7921 ggttatggca gcactgcata attctcttac tgtcatgcca tccgtaagat gcttttctgt
7981 gactggtgag tactcaacca agtcattctg agaatagtgt atgcggcgac cgagttgctc
8041 ttgcccggcg tcaatacggg ataataccgc gccacatagc agaactttaa aagtgctcat
8101 cattggaaaa cgttcttcgg ggcgaaaact ctcaaggatc ttaccgctgt tgagatccag
8161 ttcgatgtaa cccactcgtg cacccaactg atcttcagca tcttttactt tcaccagcgt
8221 ttctgggtga gcaaaaacag gaaggcaaaa tgccgcaaaa aagggaataa gggcgacacg
8281 gaaatgttga atactcatac tcttcctttt tcaatattat tgaagcattt atcagggtta
8341 ttgtctcatg agcggataca tatttgaatg tatttagaaa aataaacaaa taggggttcc
8401 gcgcacattt ccccgaaaag tgccacctga cgtc
TRE-CK:
(SEQ ID NO: 30)
      1 gacggatcgg gagatctccc gatcccctat ggtgcactct cagtacaatc tgctctgatg
    61 ccgcatagtt aagccagtat ctgctccctg cttgtgtgtt ggaggtcgct gagtagtgcg
  121 cgagcaaaat ttaagctaca acaaggcaag gcttgaccga caattgcatg aagaatctgc
  181 ttagggttag gcgttttgcg ctgcttcgcg atgtacgggc cagatatacg cgttgacatt
  241 gattattgac tagttattaa tagtaatcaa ttacggggtc attagttcat agcccatata
  301 tggagttccg cgttacataa cttacggtaa atggcccgcc tggctgaccg cccaacgacc
  361 cccgcccatt gacgtcaata atgacgtatg ttcccatagt aacgccaata gggactttcc
  421 attgacgtca atgggtggag tatttacggt aaactgccca cttggcagta catcaagtgt
  481 atcatatgcc aagtacgccc cctattgacg tcaatgacgg taaatggccc gcctggcatt
  541 atgcccagta catgacctta tgggactttc ctacttggca gtacatctac gtattagtca
  601 tcgctattac catggtgatg cggttttggc agtacatcaa tgggcgtgga tagcggtttg
  661 actcacgggg atttccaagt ctccacccca ttgacgtcaa tgggagtttg ttttggcacc
  721 aaaatcaacg ggactttcca aaatgtcgta acaactccgc cccattgacg caaatgggcg
  781 gtaggcgtgt acggtgggag gtctatataa gcagagctct ctggctaact agagaaccca
  841 ctgcttactg gcttatcgaa attaatacga ctcactatag ggagacccaa gctggctagc
  901 atgtctagac tggacaagag caaagtcata aacggcgctc tggaattact caatggagtc
  961 ggtatcgaag gcctgacgac aaggaaactc gctcaaaagc tgggagttga gcagcctacc
1021 ctgtactggc acgtgaagaa caagcgggcc ctgctcgatg ccctgccaat cgagatgctg
1081 gacaggcatc atacccactt ctgccccctg gaaggcgagt catggcaaga ctttctgcgg
1141 aacaacgcca agtcattccg ctgtgctctc ctctcacatc gcgacggggc taaagtgcat
1201 ctcggcaccc gcccaacaga gaaacagtac gaaaccctgg aaaatcagct cgcgttcctg
1261 tgtcagcaag gcttctccct ggagaacgca ctgtacgctc tgtccgccgt gggccacttt
1321 acactgggct gcgtattgga ggaacaggag catcaagtag caaaagagga aagagagaca
1381 cctaccaccg attctatgcc cccacttctg agacaagcaa ttgagctgtt cgaccggcag
1441 ggagccgaac ctgccttcct tttcggcctg gaactaatca tatgtggcct ggagaaacag
1501 ctaaagtgcg aaagcggcgg gccggccgac gcccttgacg attttgactt agacatgctc
1561 ccagccgatg cccttgacga ctttgacctt gatatgctgc ctgctgacgc tcttgacgat
1621 tttgaccttg acatgctccc cgggtaaacc cagctttctt gtacaaagtg gtgatcttaa
1681 ggagggccta tttcccatga ttccttcata tttgcatata cgatacaagg ctgttagaga
1741 gataattgga attaatttga ctgtaaacac aaagatatta gtacaaaata cgtgacgtag
1801 aaagtaataa tttcttgggt agtttgcagt tttaaaatta tgttttaaaa tggactatca
1861 tatgcttacc gtaacttgaa agtatttcga tttcttggct ttatatatct tgtggaaagg
1921 acgaaacacc gggtcttcga gaagacctgt ttaagagcta tgctggaaac agcatagcaa
1981 gtttaaataa ggctagtccg ttatcaactt gaaaaagtgg caccgagtcg gtgcctgaat
2041 gcctgcgagc atcttttttt gttttttatg tctcggatcc cgatcacgag actagcctcg
2101 agttggcttt actccctatc agtgatagag aacgtatgaa gagtttactc cctatcagtg
2161 atagagaacg tatgcagact ttactcccta tcagtgatag agaacgtata aggagtttac
2221 tccctatcag tgatagagaa cgtatgacca gtttactccc tatcagtgat agagaacgta
2281 tctacagttt actccctatc agtgatagag aacgtatatc cagtttactc cctatcagtg
2341 atagagaacg tataagcttt aggcgtgtac ggtgggcgcc tataaaagca gagctcgttt
2401 agtgaaccgt cagatcgcct ggagcaattc cacaacactt ttgtcttata ccaactttcc
2461 gtaccacttc ctaccctcgt aaaccgcggc cccgaattgc aagtttgtac aaaggtacca
2521 tgcccaaaaa gaaaagaaaa gtgggtagta tgaaatcaat tcgctgtaaa aactgcaaca
2581 aactgttatt taaggcggat agttttgatc acattgaaat caggtgtccg cgttgcaaac
2641 gtcacatcat aatgctgaat gcctgcgagc atcccacgga gaaacattgt gggaaaagag
2701 aaaaaatcac gcattctgac gaaaccgtgc gttatggagg aggtggaagc ggaggaggag
2761 gaagcggagg aggaggtagc ctcgagatgg atgctaagtc actaactgcc tggtcccgga
2821 cactggtgac cttcaaggat gtatttgtgg acttcaccag ggaggagtgg aagctgctgg
2881 acactgctca gcagatcgtg tacagaaatg tgatgctgga gaactataag aacctggttt
2941 ccttgggtta tcagcttact aagccagatg tgatcctccg gttggagaag ggagaagagc
3001 cctaggaatt ctgcagatat ccagcacagt ggcggccgct cgagtctaga gggcccgttt
3061 aaacccgctg atcagcctcg actgtgcctt ctagttgcca gccatctgtt gtttgcccct
3121 cccccgtgcc ttccttgacc ctggaaggtg ccactcccac tgtcctttcc taataaaatg
3181 aggaaattgc atcgcattgt ctgagtaggt gtcattctat tctggggggt ggggtggggc
3241 aggacagcaa gggggaggat tgggaagaca atagcaggca tgctggggat gcggtgggct
3301 ctatggcttc tgaggcggaa agaaccagct ggggctctag ggggtatccc cacgcgccct
3361 gtagcggcgc attaagcgcg gcgggtgtgg tggttacgcg cagcgtgacc gctacacttg
3421 ccagcgccct agcgcccgct cctttcgctt tcttcccttc ctttctcgcc acgttcgccg
3481 gctttccccg tcaagctcta aatcgggggc tccctttagg gttccgattt agtgctttac
3541 ggcacctcga ccccaaaaaa cttgattagg gtgatggttc acgtagtggg ccatcgccct
3601 gatagacggt ttttcgccct ttgacgttgg agtccacgtt ctttaatagt ggactcttgt
3661 tccaaactgg aacaacactc aaccctatct cggtctattc ttttgattta taagggattt
3721 tgccgatttc ggcctattgg ttaaaaaatg agctgattta acaaaaattt aacgcgaatt
3781 aattctgtgg aatgtgtgtc agttagggtg tggaaagtcc ccaggctccc cagcaggcag
3841 aagtatgcaa agcatgcatc tcaattagtc agcaaccagg tgtggaaagt ccccaggctc
3901 cccagcaggc agaagtatgc aaagcatgca tctcaattag tcagcaacca tagtcccgcc
3961 cctaactccg cccatcccgc ccctaactcc gcccagttcc gcccattctc cgccccatgg
4021 ctgactaatt ttttttattt atgcagaggc cgaggccgcc tctgcctctg agctattcca
4081 gaagtagtga ggaggctttt ttggaggcct aggcttttgc aaaaagctcc cgggagcttg
4141 tatatccatt ttcggatctg atcaagagac aggatgagga tcgtttcgca tgattgaaca
4201 agatggattg cacgcaggtt ctccggccgc ttgggtggag aggctattcg gctatgactg
4261 ggcacaacag acaatcggct gctctgatgc cgccgtgttc cggctgtcag cgcaggggcg
4321 cccggttctt tttgtcaaga ccgacctgtc cggtgccctg aatgaactgc aggacgaggc
4381 agcgcggcta tcgtggctgg ccacgacggg cgttccttgc gcagctgtgc tcgacgttgt
4441 cactgaagcg ggaagggact ggctgctatt gggcgaagtg ccggggcagg atctcctgtc
4501 atctcacctt gctcctgccg agaaagtatc catcatggct gatgcaatgc ggcggctgca
4561 tacgcttgat ccggctacct gcccattcga ccaccaagcg aaacatcgca tcgagcgagc
4621 acgtactcgg atggaagccg gtcttgtcga tcaggatgat ctggacgaag agcatcaggg
4681 gctcgcgcca gccgaactgt tcgccaggct caaggcgcgc atgcccgacg gcgaggatct
4741 cgtcgtgacc catggcgatg cctgcttgcc gaatatcatg gtggaaaatg gccgcttttc
4801 tggattcatc gactgtggcc ggctgggtgt ggcggaccgc tatcaggaca tagcgttggc
4861 tacccgtgat attgctgaag agcttggcgg cgaatgggct gaccgcttcc tcgtgcttta
4921 cggtatcgcc gctcccgatt cgcagcgcat cgccttctat cgccttcttg acgagttctt
4981 ctgagcggga ctctggggtt cgaaatgacc gaccaagcga cgcccaacct gccatcacga
5041 gatttcgatt ccaccgccgc cttctatgaa aggttgggct tcggaatcgt tttccgggac
5101 gccggctgga tgatcctcca gcgcggggat ctcatgctgg agttcttcgc ccaccccaac
5161 ttgtttattg cagcttataa tggttacaaa taaagcaata gcatcacaaa tttcacaaat
5221 aaagcatttt tttcactgca ttctagttgt ggtttgtcca aactcatcaa tgtatcttat
5281 catgtctgta taccgtcgac ctctagctag agcttggcgt aatcatggtc atagctgttt
5341 cctgtgtgaa attgttatcc gctcacaatt ccacacaaca tacgagccgg aagcataaag
5401 tgtaaagcct ggggtgccta atgagtgagc taactcacat taattgcgtt gcgctcactg
5461 cccgctttcc agtcgggaaa cctgtcgtgc cagctgcatt aatgaatcgg ccaacgcgcg
5521 gggagaggcg gtttgcgtat tgggcgctct tccgcttcct cgctcactga ctcgctgcgc
5581 tcggtcgttc ggctgcggcg agcggtatca gctcactcaa aggcggtaat acggttatcc
5641 acagaatcag gggataacgc aggaaagaac atgtgagcaa aaggccagca aaaggccagg
5701 aaccgtaaaa aggccgcgtt gctggcgttt ttccataggc tccgcccccc tgacgagcat
5761 cacaaaaatc gacgctcaag tcagaggtgg cgaaacccga caggactata aagataccag
5821 gcgtttcccc ctggaagctc cctcgtgcgc tctcctgttc cgaccctgcc gcttaccgga
5881 tacctgtccg cctttctccc ttcgggaagc gtggcgcttt ctcatagctc acgctgtagg
5941 tatctcagtt cggtgtaggt cgttcgctcc aagctgggct gtgtgcacga accccccgtt
6001 cagcccgacc gctgcgcctt atccggtaac tatcgtcttg agtccaaccc ggtaagacac
6061 gacttatcgc cactggcagc agccactggt aacaggatta gcagagcgag gtatgtaggc
6121 ggtgctacag agttcttgaa gtggtggcct aactacggct acactagaag aacagtattt
6181 ggtatctgcg ctctgctgaa gccagttacc ttcggaaaaa gagttggtag ctcttgatcc
6241 ggcaaacaaa ccaccgctgg tagcggtttt tttgtttgca agcagcagat tacgcgcaga
6301 aaaaaaggat ctcaagaaga tcctttgatc ttttctacgg ggtctgacgc tcagtggaac
6361 gaaaactcac gttaagggat tttggtcatg agattatcaa aaaggatctt cacctagatc
6421 cttttaaatt aaaaatgaag ttttaaatca atctaaagta tatatgagta aacttggtct
6481 gacagttacc aatgcttaat cagtgaggca cctatctcag cgatctgtct atttcgttca
6541 tccatagttg cctgactccc cgtcgtgtag ataactacga tacgggaggg cttaccatct
6601 ggccccagtg ctgcaatgat accgcgagac ccacgctcac cggctccaga tttatcagca
6661 ataaaccagc cagccggaag ggccgagcgc agaagtggtc ctgcaacttt atccgcctcc
6721 atccagtcta ttaattgttg ccgggaagct agagtaagta gttcgccagt taatagtttg
6781 cgcaacgttg ttgccattgc tacaggcatc gtggtgtcac gctcgtcgtt tggtatggct
6841 tcattcagct ccggttccca acgatcaagg cgagttacat gatcccccat gttgtgcaaa
6901 aaagcggtta gctccttcgg tcctccgatc gttgtcagaa gtaagttggc cgcagtgtta
6961 tcactcatgg ttatggcagc actgcataat tctcttactg tcatgccatc cgtaagatgc
7021 ttttctgtga ctggtgagta ctcaaccaag tcattctgag aatagtgtat gcggcgaccg
7081 agttgctctt gcccggcgtc aatacgggat aataccgcgc cacatagcag aactttaaaa
7141 gtgctcatca ttggaaaacg ttcttcgggg cgaaaactct caaggatctt accgctgttg
7201 agatccagtt cgatgtaacc cactcgtgca cccaactgat cttcagcatc ttttactttc
7261 accagcgttt ctgggtgagc aaaaacagga aggcaaaatg ccgcaaaaaa gggaataagg
7321 gcgacacgga aatgttgaat actcatactc ttcctttttc aatattattg aagcatttat
7381 cagggttatt gtctcatgag cggatacata tttgaatgta tttagaaaaa taaacaaata
7441 ggggttccgc gcacatttcc ccgaaaagtg ccacctgacg tc
```

Traffic light reporter (TLR) plasmid construction: TLR construct was assembled with a nonfunctional EGFP variant (bf-Venus) where codons 53-63 were disrupted, a T2A peptide, and a red fluorescent gene that has a 2-bp shifted reading frame (fs-mCherry)(Certo, M. T. et al. (2011) Nature Methods 8, 671-U102, doi:10.1038/Nmeth. 1648). The expression cassette of Venus-T2A-mCherry was cloned in between the CMV promoter and SV40 poly (A) signal. The CRISPR targeting site was designed at the bf-Venus disrupted region. As Cas9 specifically induces DSBs, if DSBs are repaired by the NHEJ pathway, approximately 1/3 of the repaired events would generate in-frame functional mCherry. Alternatively, if DSBs are repaired by the EGFP HDR donor to generate intact Venus, the disrupted region of bf-Venus would be corrected leaving fs-mCherry remaining out of frame.

```
TLR DNA sequence (SEQ ID NO: 31):
atggtgagcaagggcgaggagctgttcaccggggtggtgcccatcctggtcgagctggacggcgacgtaaacggccacaagt
tcagcgtgtccggcgagggcgagggcgatgccacctacggcaagctgaccctgaagttcatctgcaccaccggcaacctgca
gggagcagcgtcttcgagagtgaggacactagtgtgaaccctgacctacggcgtgcagtgcttcagccgctaccccgaccac
atgaagcagcacgacttcttcaagtccgccatgcccgaaggctacgtccaggagcgcaccatcttcttcaaggacgacggcaac
tacaagacccgcgccgaggtgaagttcgagggcgacaccctggtgaaccgcatcgagctgaagggcatcgacttcaaggag
gacggcaacatcctggggcacaagctggagtacaactacaacagccacaacgtctatatcatggccgacaagcagaagaacg
gcatcaaggtgaacttcaagatccgccacaacatcgaggacggcagcgtgcagctcgccgaccactaccagcagaacacccc
catcggcgacggccccgtgctgctgcccgacaaccactacctgagcacccagtccgccctgagcaaagaccccaacgagaa
gcgcgatcacatggtcctgctggagttcgtgaccgccgccgggatcactctcggcatggacgagctgtacaagtaaGAATT
CcgGAGGGCAGAGGAAGTCTGCTAACATGCGGTGACGTCGAGGAGAATCCTGGCC
CAGGATCCgtgagcaagggcgaggaggataactccgccatcatcaaggagttcctgcgcttcaaggtgcacatggagg
gctccgtgaacggccacgagttcgagatcgagggcgagggcgagggccgcccctacgagggcacccagaccgccaagctg
aaggtgaccaagggtggccccctgccatcgcctgggacatcctgtcccctcagttcatgtacggctccaaggcctacgtgaag
caccccgccgacatccccgactacttgaagctgtccttccccgagggcttcaagtgggagcgcgtgatgaacttcgaggacgg
cggcgtggtgaccgtgacccaggactcctctctgcaggacggcgagttcatctacaaggtgaagctgcgcggcaccaacttcc
cctccgacggccccgtaatgcagaagaagaccatgggctgggaggcctcctccgagcggatgtaccccgaggacggcgccc
tgaagggcgagatcaagcagaggctgaagctgaaggacggcggccactacgacgctgaggtcaagaccacctacaaggcc
aagaagcccgtgcagctgcccggcgcctacaacgtcaacatcaagttggacatcacctcccacaacgaggactacaccatcgt
ggaacagtacgaacgcgccgagggccgccactccaccggcggcatggacgagctgtacaagtga
Venus, CRISPR targeting site, T2A, mCherry
Traffic light plasmid:
(SEQ ID NO: 32)
      1 tagttattaa tagtaatcaa ttacggggtc attagttcat agcccatata tggagttccg
    61 cgttacataa cttacggtaa atggcccgcc tggctgaccg cccaacgacc cccgcccatt
  121 gacgtcaata atgacgtatg ttcccatagt aacgccaata gggactttcc attgacgtca
  181 atgggtggag tatttacggt aaactgccca cttggcagta catcaagtgt atcatatgcc
  241 aagtacgccc cctattgacg tcaatgacgg taaatggccc gcctggcatt atgcccagta
  301 catgacctta tgggactttc ctacttggca gtacatctac gtattagtca tcgctattac
  361 catggtgatg cggttttggc agtacatcaa tgggcgtgga tagcggtttg actcacgggg
  421 atttccaagt ctccacccca ttgacgtcaa tgggagtttg ttttggcacc aaaatcaacg
  481 ggactttcca aaatgtcgta acaactccgc cccattgacg caaatgggcg gtaggcgtgt
  541 acggtgggag gtctatataa gcagagctgg tttagtgaac cgtcagatcc gctagcgcta
  601 ccggactcag atctatggtg agcaagggcg aggagctgtt caccggggtg gtgcccatcc
  661 tggtcgagct ggacggcgac gtaaacggcc acaagttcag cgtgtccggc gagggcgagg
  721 gcgatgccac ctacggcaag ctgaccctga agttcatctg caccaccggc aacctgcagg
  781 gagcagcgtc ttcgagagtg aggacactag tgtgaaccct gacctacggc gtgcagtgct
  841 tcagccgcta ccccgaccac atgaagcagc acgacttctt caagtccgcc atgcccgaag
  901 gctacgtcca ggagcgcacc atcttcttca aggacgacgg caactacaag acccgcgccg
  961 aggtgaagtt cgagggcgac accctggtga accgcatcga gctgaagggc atcgacttca
1021 aggaggacgg caacatcctg gggcacaagc tggagtacaa ctacaacagc cacaacgtct
1081 atatcatggc cgacaagcag aagaacggca tcaaggtgaa cttcaagatc cgccacaaca
1141 tcgaggacgg cagcgtgcag ctcgccgacc actaccagca gaacaccccc atcggcgacg
1201 gccccgtgct gctgcccgac aaccactacc tgagcaccca gtccgccctg agcaaagacc
1261 ccaacgagaa gcgcgatcac atggtcctgc tggagttcgt gaccgccgcc gggatcactc
1321 tcggcatgga cgagctgtac aagtaagaat tccggagggc agaggaagtc tgctaacatg
1381 cggtgacgtc gaggagaatc ctggcccagg atccgtgagc aagggcgagg aggataactc
1441 cgccatcatc aaggagttcc tgcgcttcaa ggtgcacatg gagggctccg tgaacggcca
1501 cgagttcgag atcgagggcg agggcgaggg ccgcccctac gagggcaccc agaccgccaa
1561 gctgaaggtg accaagggtg gccccctgcc cttcgcctgg gacatcctgt cccctcagtt
1621 catgtacggc tccaaggcct acgtgaagca ccccgccgac atccccgact acttgaagct
1681 gtccttcccc gagggcttca agtgggagcg cgtgatgaac ttcgaggacg gcggcgtggt
1741 gaccgtgacc caggactcct ctctgcagga cggcgagttc atctacaagg tgaagctgcg
1801 cggcaccaac ttcccctccg acggccccgt aatgcagaag aagaccatgg gctgggaggc
1861 ctcctccgag cggatgtacc ccgaggacgg cgccctgaag ggcgagatca agcagaggct
1921 gaagctgaag gacggcggcc actacgacgc tgaggtcaag accacctaca aggccaagaa
1981 gcccgtgcag ctgcccggcg cctacaacgt caacatcaag ttggacatca cctcccacaa
2041 cgaggactac accatcgtgg aacagtacga acgcgccgag ggccgccact ccaccggcgg
2101 catggacgag ctgtacaagt gagcggccgc gactctagat cataatcagc cataccacat
2161 ttgtagaggt tttacttgct ttaaaaaacc tcccacacct ccccctgaac ctgaaacata
2221 aaatgaatgc aattgttgtt gttaacttgt ttattgcagc ttataatggt tacaaataaa
2281 gcaatagcat cacaaatttc acaaataaag catttttttc actgcattct agttgtggtt
2341 tgtccaaact catcaatgta tcttaaggcg taaattgtaa gcgttaatat tttgttaaaa
2401 ttcgcgttaa atttttgtta aatcagctca ttttttaacc aataggccga aatcggcaaa
2461 atcccttata aatcaaaaga atagaccgag atagggttga gtgttgttcc agtttggaac
2521 aagagtccac tattaaagaa cgtggactcc aacgtcaaag ggcgaaaaac cgtctatcag
2581 ggcgatggcc cactacgtga accatcaccc taatcaagtt ttttggggtc gaggtgccgt
2641 aaagcactaa atcggaaccc taaagggagc ccccgattta gagcttgacg gggaaagccg
2701 gcgaacgtgg cgagaaagga agggaagaaa gcgaaaggag cgggcgctag ggcgctggca
2761 agtgtagcgg tcacgctgcg cgtaaccacc acacccgccg cgcttaatgc gccgctacag
2821 ggcgcgtcag gtggcacttt tcggggaaat gtgcgcggaa cccctatttg tttatttttc
2881 taaatacatt caaatatgta tccgctcatg agacaataac cctgataaat gcttcaataa
2941 tattgaaaaa ggaagagtcc tgaggcggaa agaaccagct gtggaatgtg tgtcagttag
3001 ggtgtggaaa gtccccaggc tccccagcag gcagaagtat gcaaagcatg catctcaatt
3061 agtcagcaac caggtgtgga aagtccccag gctccccagc aggcagaagt atgcaaagca
3121 tgcatctcaa ttagtcagca accatagtcc cgcccctaac tccgcccatc ccgcccctaa
3181 ctccgcccag ttccgcccat tctccgcccc atggctgact aatttttttt atttatgcag
3241 aggccgaggc cgcctcggcc tctgagctat tccagaagta gtgaggaggc ttttttggag
3301 gcctaggctt ttgcaaagat cgatcaagag acaggatgag gatcgtttcg catgattgaa
3361 caagatggat tgcacgcagg ttctccggcc gcttgggtgg agaggctatt cggctatgac
3421 tgggcacaac agacaatcgg ctgctctgat gccgccgtgt tccggctgtc agcgcagggg
3481 cgcccggttc tttttgtcaa gaccgacctg tccggtgccc tgaatgaact gcaagacgag
3541 gcagcgcggc tatcgtggct ggccacgacg ggcgttcctt gcgcagctgt gctcgacgtt
3601 gtcactgaag cgggaaggga ctggctgcta ttgggcgaag tgccggggca ggatctcctg
3661 tcatctcacc ttgctcctgc cgagaaagta tccatcatgg ctgatgcaat gcggcggctg
3721 catacgcttg atccggctac ctgcccattc gaccaccaag cgaaacatcg catcgagcga
3781 gcacgtactc ggatggaagc cggtcttgtc gatcaggatg atctggacga agagcatcag
3841 gggctcgcgc cagccgaact gttcgccagg ctcaaggcga gcatgcccga cggcgaggat
3901 ctcgtcgtga cccatggcga tgcctgcttg ccgaatatca tggtggaaaa tggccgcttt
3961 tctggattca tcgactgtgg ccggctgggt gtggcggacc gctatcagga catagcgttg
4021 gctacccgtg atattgctga agagcttggc ggcgaatggg ctgaccgctt cctcgtgctt
4081 tacggtatcg ccgctcccga ttcgcagcgc atcgccttct atcgccttct tgacgagttc
4141 ttctgagcgg gactctgggg ttcgaaatga ccgaccaagc gacgcccaac ctgccatcac
4201 gagatttcga ttccaccgcc gccttctatg aaaggttggg cttcggaatc gttttccggg
4261 acgccggctg gatgatcctc cagcgcgggg atctcatgct ggagttcttc gcccacccta
4321 gggggaggct aactgaaaca cggaaggaga caataccgga aggaacccgc gctatgacgg
4381 caataaaaag acagaataaa acgcacggtg ttgggtcgtt tgttcataaa cgcggggttc
4441 ggtcccaggg ctggcactct gtcgataccc caccgagacc ccattggggc caatacgccc
4501 gcgtttcttc cttttcccca ccccaccccc caagttcggg tgaaggccca gggctcgcag
4561 ccaacgtcgg ggcggcaggc cctgccatag cctcaggtta ctcatatata ctttagattg
4621 atttaaaact tcatttttaa tttaaaagga tctaggtgaa gatccttttt gataatctca
4681 tgaccaaaat cccttaacgt gagttttcgt tccactgagc gtcagacccc gtagaaaaga
4741 tcaaaggatc ttcttgagat cctttttttc tgcgcgtaat ctgctgcttg caaacaaaaa
4801 aaccaccgct accagcggtg gtttgtttgc cggatcaaga gctaccaact ctttttccga
4861 aggtaactgg cttcagcaga gcgcagatac caaatactgt ccttctagtg tagccgtagt
4921 taggccacca cttcaagaac tctgtagcac cgcctacata cctcgctctg ctaatcctgt
4981 taccagtggc tgctgccagt ggcgataagt cgtgtcttac cgggttggac tcaagacgat
5041 agttaccgga taaggcgcag cggtcgggct gaacgggggg ttcgtgcaca cagcccagct
5101 tggagcgaac gacctacacc gaactgagat acctacagcg tgagctatga gaaagcgcca
5161 cgcttcccga agggagaaag gcggacaggt atccggtaag cggcagggtc ggaacaggag
5221 agcgcacgag ggagcttcca gggggaaacg cctggtatct ttatagtcct gtcgggtttc
5281 gccacctctg acttgagcgt cgatttttgt gatgctcgtc aggggggcgg agcctatgga
5341 aaaacgccag caacgcggcc tttttacggt tcctggcctt ttgctggcct tttgctcaca
5401 tgttctttcc tgcgttatcc cctgattctg tggataaccg tattaccgcc atgcat
AAVS1 HDR donor DNA sequence
(SEQ ID NO: 33)
ttctccttctggggcctgtgccatctctcgtttcttaggatggccttctccgacggatgtctcccttgcgtcccgcctccccttcttgta
ggcctgcatcatcaccgtttttctggacaaccccaaagtaccccgtctccctggctttagccacctctccatcctcttgctttctttgcc
tggacaccccgttctcctgtggattcgggtcacctctcactcctttcatttgggcagctcccctaccccccttacctctctagtctgtg
ctagctcttccagccccctgtcatggcatcttccaggggtccgagagctcagctagtcttcttcctccaacccgggcccctatgtcc
acttcaggacagcatgtttgctgcctccagggatcctgtgtccccgagctgggaccaccttatattcccagggccggttaatgtgg
ctctggttctgggtacttttatctgtcccctccaccccacagtggggggtaccagtcgatccaacatggcgacttgtcccatcccc
ggcatgtttaaatatactaattattcttgaactaattttaatcaaccgatttatctctatccgcaggtggcggaggttccggtgga
agcggaggtagcggcggatccgagggccgcggcagcctgctgacctgcggcgatgtggaggagaaccccgggcccAT
GGTGAGCAAGGGCGAGGAGCTGTTCACCGGGGTGGTGCCCATCCTGGTCGAGCT
GGACGGCGACGTAAACGGCCACAAGTTCAGCGTGTCCGGCGAGGGCGAGGGCGA
TGCCACCTACGGCAAGCTGACCCTGAAGTTCATCTGCACCACCGGCAAGCTGCCC
GTGCCCTGGCCCACCCTCGTGACCACCCTGACCTACGGCGTGCAGTGCTTCAGCC
GCTACCCCGACCACATGAAGCAGCACGACTTCTTCAAGTCCGCCATGCCCGAAGG
CTACGTCCAGGAGCGCACCATCTTCTTCAAGGACGACGGCAACTACAAGACCCGC
GCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACA
ACAGCCACAACGTCTATATCATGGCCGACAAGCAGAAGAACGGCATCAAGGTGAA
CTTCAAGATCCGCCACAACATCGAGGACGGCAGCGTGCAGCTCGCCGACCACTAC
CAGCAGAACACCCCCATCGGCGACGGCCCCGTGCTGCTGCCCGACAACCACTAC
CTGAGCACCCAGTCCGCCCTGAGCAAAGACCCCAACGAGAAGCGCGATCACATGG
TCCTGCTGGAGTTCGTGACCGCCGCCGGGATCACTCTCGGCATGGACGAGCTGTA
CAAGTAAAATAAAAGATCTTTATTTTCATTAGATCTGTGTGTTGGTTTTTTGTGTgaattc
ccactagggacaggattggtgacagaaaagccccatccttaggcctcctccttcctagtctcctgatattgggtctaaccc
ccacctcctgttaggcagattccttatctggtgacacacccccatttcctggagccatctctctccttgccagaacctctaag
gtttgcttacgatggagccagagaggatcctgggagggagagcttggcagggggtgggagggaagggggggatgcgt
gacctgcccggttctcagtggccaccctgcgctaccctctcccagaacctgagctgctctgacgcggctgtctggtgcgttt
cactgatcctggtgctgcagcttccttacacttcccaagaggagaagcagtttggaaaaacaaaatcagaataagttggt
cctgagttctaactttggctcttcacctttctagtccccaatttatattgttcctccgtgcgtcagttttacctgtgagataagg
ccagtagccagccccgtcctggcagggctgtggtgaggaggggggtgtccgtgtggaaaactccctttgtgagaatggt
gcgtcctaggtgttcaccaggtcgtggccgcctctactccctttctctttctccatccttctttccttaaagagtccccagtgct
atctgggacatattcctccgcccagagcagggtcccgcttccctaaggccctgctctgggcttctgggtttgagtccttggc
aagcccaggagaggcgctcaggcttccctgtcccccttcctcgtccaccatctcatgcccctggctctcctgccccttccct
acaggggttcctggctctgctcttcagactgagccccgt
Left homology arm of AAVS1, SA-T2A-EGF P-ShortPA, Right homology arm of AAVS1
ACTB HDR donor DNA sequence
(SEQ ID NO: 34)
CGGCTCTGCCTGACATGAGGGTTACCCCTCGGGGCTGTGCTGTGGAAGCTAA
GTCCTGCCCTCATTTCCCTCTCAGGCATGGAGTCCTGTGGCATCCACGAAACT
ACCTTCAACTCCATCATGAAGTGTGACGTGGACATCCGCAAAGACCTGTACG
CCAACACAGTGCTGTCTGGCGGCACCACCATGTACCCTGGCATTGCCGACAG
GATGCAGAAGGAGATCACTGCCCTGGCACCCAGCACAATGAAGATCAAGGTG
GGTGTCTTTCCTGCCTGAGCTGACCTGGGCAGGTCGGCTGTGGGGTCCTGTGG
TGTGTGGGGAGCTGTCACATCCAGGGTCCTCACTGCCTGTCCCCTTCCCTCCT
CAGATCATTGCTCCTCCTGAGCGCAAGTACTCCGTGTGGATCGGCGGCTCCAT
CCTGGCCTCGCTGTCCACCTTCCAGCAGATGTGGATCAGCAAGCAGGAGTAT
GACGAGTCCGGCCCCTCCATCGTCCACCGCAAATGCTTCgagggccgcggcagcctgctg
acctgcggcgatgtggaggagaaccccgggcccATGGTGAGCAAGGGCGAGGAGCTGTTCACCG
GGGTGGTGCCCATCCTGGTCGAGCTGGACGGCGACGTAAACGGCCACAAGTTCA
GCGTGTCCGGCGAGGGCGAGGGCGATGCCACCTACGGCAAGCTGACCCTGAAGT
TCATCTGCACCACCGGCAAGCTGCCCGTGCCCTGGCCCACCCTCGTGACCACCCT
GACCTACGGCGTGCAGTGCTTCAGCCGCTACCCCGACCACATGAAGCAGCACGAC
TTCTTCAAGTCCGCCATGCCCGAAGGCTACGTCCAGGAGCGCACCATCTTCTTCAA
GGACGACGGCAACTACAAGACCCGCGCCGAGGTGAAGTTCGAGGGCGACACCCT
GGTGAACCGCATCGAGCTGAAGGGCATCGACTTCAAGGAGGACGGCAACATCCTG
GGGCACAAGCTGGAGTACAACTACAACAGCCACAACGTCTATATCATGGCCGACAA
GCAGAAGAACGGCATCAAGGTGAACTTCAAGATCCGCCACAACATCGAGGACGGC
AGCGTGCAGCTCGCCGACCACTACCAGCAGAACACCCCCATCGGCGACGGCCCC
GTGCTGCTGCCCGACAACCACTACCTGAGCACCCAGTCCGCCCTGAGCAAAGACC
CCAACGAGAAGCGCGATCACATGGTCCTGCTGGAGTTCGTGACCGCCGCCGGGAT
CACTCTCGGCATGGACGAGCTGTACAAGTAAtaggcggactatgacttagttgcgttacaccctttcttga
caaaacctaacttgcgcagaaaacaagatgagattggcatggctttatttgttttttttgttttgttttggttttttttttttttttggcttgactc
aggatttaaaaactggaacggtgaaggtgacagcagtcggttggagcgagcatcccccaaagttcacaatgtggccgaggactt
tgattgcacattgttgtttttttaatagtcattccaaatatgagatgcgttgttacaggaagtcccttgccatcctaaaagccaccccact
tctctctaaggagaatggcccagtcctctcccaagtccacacaggggaggtgatagcattgctttcgtgtaaattatgtaatgcaaa
atttttttaatcttcgccttaatacttttttattttgttttattttgaatgatgagccttcgtgcccccccttcccccttttttgtcccccaacttg
agatgtatgaaggcttttggtctccctgggagtgggtggaggcagccagggcttacctgtacactgacttgagaccagttgaataa
aagtgcacaccttaaaaatgaggccaagtgtgactttgtggtgtggctgggttgggggcagcagagggtgaaccctgcaggag
ggtgaaccctgcaaaagggtggggcagtgggggccaacttgtccttacccagagtgcaggtgtgtggagatccctcctgccttg
acattgagcagccttagagggtgggggaggctcaggggtcaggtctctgttcctgcttattgggga
Left homology arm of ACTB, T2A-EGFP, Right homology arm of A CTB
sgVenus-ECFP expression plasmid (PUC57-U6-venus sgRNA-CMV-ECFP.gb):
(SEQ ID NO: 35)
      1 gacgaaaggg cctcgtgata cgcctatttt tataggttaa tgtcatgata ataatggttt
    61 cttagacgtc aggtggcact tttcggggaa atgtgcgcgg aacccctatt tgtttatttt
  121 tctaaataca ttcaaatatg tatccgctca tgagacaata accctgataa atgcttcaat
  181 aatattgaaa aaggaagagt atgagtattc aacatttccg tgtcgccctt attccctttt
  241 ttgcggcatt ttgccttcct gtttttgctc acccagaaac gctggtgaaa gtaaaagatg
  301 ctgaagatca gttgggtgca cgagtgggtt acatcgaact ggatctcaac agcggtaaga
  361 tccttgagag ttttcgcccc gaagaacgtt ttccaatgat gagcactttt aaagttctgc
  421 tatgtggcgc ggtattatcc cgtattgacg ccgggcaaga gcaactcggt cgccgcatac
  481 actattctca gaatgacttg gttgagtact caccagtcac agaaaagcat cttacggatg
  541 gcatgacagt aagagaatta tgcagtgctg ccataaccat gagtgataac actgcggcca
  601 acttacttct gacaacgatc ggaggaccga aggagctaac cgcttttttg cacaacatgg
  661 gggatcatgt aactcgcctt gatcgttggg aaccggagct gaatgaagcc ataccaaacg
  721 acgagcgtga caccacgatg cctgtagcaa tggcaacaac gttgcgcaaa ctattaactg
  781 gcgaactact tactctagct tcccggcaac aattaataga ctggatggag gcggataaag
  841 ttgcaggacc acttctgcgc tcggcccttc cggctggctg gtttattgct gataaatctg
  901 gagccggtga gcgtgggtct cgcggtatca ttgcagcact ggggccagat ggtaagccct
  961 cccgtatcgt agttatctac acgacgggga gtcaggcaac tatggatgaa cgaaatagac
1021 agatcgctga gataggtgcc tcactgatta agcattggta actgtcagac caagtttact
1081 catatatact ttagattgat ttaaaacttc atttttaatt taaaaggatc taggtgaaga
1141 tcctttttga taatctcatg accaaaatcc cttaacgtga gttttcgttc cactgagcgt
1201 cagaccccgt agaaaagatc aaaggatctt cttgagatcc tttttttctg cgcgtaatct
1261 gctgcttgca aacaaaaaaa ccaccgctac cagcggtggt ttgtttgccg gatcaagagc
1321 taccaactct ttttccgaag gtaactggct tcagcagagc gcagatacca aatactgttc
1381 ttctagtgta gccgtagtta ggccaccact tcaagaactc tgtagcaccg cctacatacc
1441 tcgctctgct aatcctgtta ccagtggctg ctgccagtgg cgataagtcg tgtcttaccg
1501 ggttggactc aagacgatag ttaccggata aggcgcagcg gtcgggctga acggggggtt
1561 cgtgcacaca gcccagcttg gagcgaacga cctacaccga actgagatac ctacagcgtg
1621 agctatgaga aagcgccacg cttcccgaag ggagaaaggc ggacaggtat ccggtaagcg
1681 gcagggtcgg aacaggagag cgcacgaggg agcttccagg gggaaacgcc tggtatcttt
1741 atagtcctgt cgggtttcgc cacctctgac ttgagcgtcg atttttgtga tgctcgtcag
1801 gggggcggag cctatggaaa aacgccagca acgcggcctt tttacggttc ctggcctttt
1861 gctggccttt tgctcacatg ttctttcctg cgttatcccc tgattctgtg gataaccgta
1921 ttaccgcctt tgagtgagct gataccgctc gccgcagccg aacgaccgag cgcagcgagt
1981 cagtgagcga ggaagcggaa gagcgcccaa tacgcaaacc gcctctcccc gcgcgttggc
2041 cgattcatta atgcagctgg cacgacaggt ttcccgactg gaaagcgggc agtgagcgca
2101 acgcaattaa tgtgagttag ctcactcatt aggcacccca ggctttacac tttatgcttc
2161 cggctcgtat gttgtgtgga attgtgagcg gataacaatt tcacacagga aacagctatg
2221 accatgatta cgccaagctt gggcgttaca taacttacgg taaatggccc gcctggctga
2281 ccgcccaacg acccccgccc attgacgtca ataatgacgt atgttcccat agtaacgcca
2341 atagggactt tccattgacg tcaatgggtg gagtatttac ggtaaactgc ccacttggca
2401 gtacatcaag tgtatcatat gccaagtacg ccccctattg acgtcaatga cggtaaatgg
2461 cccgcctggc attatgccca gtacatgacc ttatgggact ttcctacttg gcagtacatc
2521 tacgtattag tcatcgctat taccatggtg atgcggtttt ggcagtacat caatgggcgt
2581 ggatagcggt ttgactcacg gggatttcca agtctccacc ccattgacgt caatgggagt
2641 ttgttttggc accaaaatca acgggacttt ccaaaatgtc gtaacaactc cgccccattg
2701 acgcaaatgg gcggtaggcg tgtacggtgg gaggtctata taagcagagc tggtttagtg
2761 aaccgtcaga tccgctagcg ctaccggtcg ccaccatggt gagcaagggc gaggagctgt
2821 tcaccggggt ggtgcccatc ctggtcgagc tggacggcga cgtaaacggc cacaagttca
2881 gcgtgtccgg cgagggcgag ggcgatgcca cctacggcaa gctgaccctg aagttcatct
2941 gcaccaccgg caagctgccc gtgccctggc ccaccctcgt gaccaccctg acctggggcg
3001 tgcagtgctt cagccgctac cccgaccaca tgaagcagca cgacttcttc aagtccgcca
3061 tgcccgaagg ctacgtccag gagcgcacca tcttcttcaa ggacgacggc aactacaaga
3121 cccgcgccga ggtgaagttc gagggcgaca ccctggtgaa ccgcatcgag ctgaagggca
3181 tcgacttcaa ggaggacggc aacatcctgg ggcacaagct ggagtacaac tacatcagcc
3241 acaacgtcta tatcaccgcc gacaagcaga agaacggcat caaggccaac ttcaagatcc
3301 gccacaacat cgaggacggc agcgtgcagc tcgccgacca ctaccagcag aacaccccca
3361 tcggcgacgg ccccgtgctg ctgcccgaca accactacct gagcacccag tccgccctga
3421 gcaaagaccc caacgagaag cgcgatcaca tggtcctgct ggagttcgtg accgccgccg
3481 ggatcactct cggcatggac gagctgtaca agtaacggga tccgatggaa cactagtgag
3541 ggcctatttc ccatgattcc ttcatatttg catatacgat acaaggctgt tagagagata
3601 attggaatta atttgactgt aaacacaaag atattagtac aaaatacgtg acgtagaaag
3661 taataatttc ttgggtagtt tgcagtttta aaattatgtt ttaaaatgga ctatcatatg
3721 cttaccgtaa cttgaaagta tttcgatttc ttggctttat atatcttgtg gaaaggacga
3781 aacaccgggt cttcgagaag acctgtttta gagctagaaa tagcaagtta aaataaggct
3841 agtccgttat caacttgaaa aagtggcacc gagtcggtgc ttttttgttt tctcgaggga
3901 acatctagat gcattcgcga ggtaccgagc tcgaattcac tggccgtcgt tttacaacgt
3961 cgtgactggg aaaaccctgg cgttacccaa cttaatcgcc ttgcagcaca tccccctttc
4021 gccagctggc gtaatagcga agaggcccgc accgatcgcc cttcccaaca gttgcgcagc
4081 ctgaatggcg aatggcgcct gatgcggtat tttctcctta cgcatctgtg cggtatttca
4141 caccgcatat ggtgcactct cagtacaatc tgctctgatg ccgcatagtt aagccagccc
4201 cgacacccgc caacacccgc tgacgcgccc tgacgggctt gtctgctccc ggcatccgct
4261 tacagacaag ctgtgaccgt ctccgggagc tgcatgtgtc agaggttttc accgtcatca
4321 ccgaaacgcg cga
Actb-F-T2A-GFP-R.gb
(SEQ ID NO: 36)
      1 atggatgatg atatcgccgc gctcgtcgtc gacaacggct ccggcatgtg caaggccggc
    61 ttcgcgggcg acgatgcccc ccgggccgtc ttcccctcca tcgtggggcg ccccaggcac
  121 caggtagggg agctggctgg gtggggcagc cccgggagcg ggcgggaggc aagggcgctt
  181 tctctgcaca ggagcctccc ggtttccggg gtgggggctg cgcccgtgct cagggcttct
  241 tgtcctttcc ttcccagggc gtgatggtgg gcatgggtca gaaggattcc tatgtgggcg
  301 acgaggccca gagcaagaga ggcatcctca ccctgaagta ccccatcgag cacggcatcg
  361 tcaccaactg ggacgacatg gagaaaatct ggcaccacac cttctacaat gagctgcgtg
  421 tggctcccga ggagcacccc gtgctgctga ccgaggcccc cctgaacccc aaggccaacc
  481 gcgagaagat gacccaggtg agtggcccgc tacctcttct ggtggccgcc tccctccttc
  541 ctggcctccc ggagctgcgc cctttctcac tggttctctc ttctgccgtt ttccgtagga
  601 ctctcttctc tgacctgagt ctcctttgga actctgcagg ttctatttgc tttttcccag
  661 atgagctctt tttctggtgt ttgtctctct gactaggtgt ctaagacagt gttgtgggtg
  721 taggtactaa cactggctcg tgtgacaagg ccatgaggct ggtgtaaagc ggccttggag
  781 tgtgtattaa gtaggtgcac agtaggtctg aacagactcc ccatcccaag accccagcac
  841 acttagccgt gttctttgca ctttctgcat gtcccccgtc tggcctggct gtccccagtg
  901 gcttccccag tgtgacatgg tgtatctctg ccttacagat catgtttgag accttcaaca
  961 ccccagccat gtacgttgct atccaggctg tgctatccct gtacgcctct ggccgtacca
1021 ctggcatcgt gatggactcc ggtgacgggg tcacccacac tgtgcccatc tacgaggggt
1081 atgccctccc ccatgccatc ctgcgtctgg acctggctgg ccgggacctg actgactacc
1141 tcatgaagat cctcaccgag cgcggctaca gcttcaccac cacggccgag cgggaaatcg
1201 tgcgtgacat taaggagaag ctgtgctacg tcgccctgga cttcgagcaa gagatggcca
1261 cggctgcttc cagctcctcc ctggagaaga gctacgagct gcctgacggc caggtcatca
1321 ccattggcaa tgagcggttc cgctgccctg aggcactctt ccagccttcc ttcctgggtg
1381 agtggagact gtctcccggc tctgcctgac atgagggtta cccctcgggg ctgtgctgtg
1441 gaagctaagt cctgccctca tttccctctc aggcatggag tcctgtggca tccacgaaac
1501 taccttcaac tccatcatga agtgtgacgt ggacatccgc aaagacctgt acgccaacac
1561 agtgctgtct ggcggcacca ccatgtaccc tggcattgcc gacaggatgc agaaggagat
1621 cactgccctg gcacccagca caatgaagat caaggtgggt gtctttcctg cctgagctga
1681 cctgggcagg tcggctgtgg ggtcctgtgg tgtgtgggga gctgtcacat ccagggtcct
1741 cactgcctgt ccccttccct cctcagatca ttgctcctcc tgagcgcaag tactccgtgt
1801 ggatcggcgg ctccatcctg gcctcgctgt ccaccttcca gcagatgtgg atcagcaagc
1861 aggagtatga cgagtccggc ccctccatcg tccaccgcaa atgcttcgag ggccgcggca
1921 gcctgctgac ctgcggcgat gtggaggaga accccgggcc catggtgagc aagggcgagg
1981 agctgttcac cggggtggtg cccatcctgg tcgagctgga cggcgacgta aacggccaca
2041 agttcagcgt gtccggcgag ggcgagggcg atgccaccta cggcaagctg accctgaagt
2101 tcatctgcac caccggcaag ctgcccgtgc cctggcccac cctcgtgacc accctgacct
2161 acggcgtgca gtgcttcagc cgctaccccg accacatgaa gcagcacgac ttcttcaagt
2221 ccgccatgcc cgaaggctac gtccaggagc gcaccatctt cttcaaggac gacggcaact
2281 acaagacccg cgccgaggtg aagttcgagg gcgacaccct ggtgaaccgc atcgagctga
2341 agggcatcga cttcaaggag gacggcaaca tcctggggca caagctggag tacaactaca
2401 acagccacaa cgtctatatc atggccgaca agcagaagaa cggcatcaag gtgaacttca
2461 agatccgcca caacatcgag gacggcagcg tgcagctcgc cgaccactac cagcagaaca
2521 cccccatcgg cgacggcccc gtgctgctgc ccgacaacca ctacctgagc acccagtccg
2581 ccctgagcaa agaccccaac gagaagcgcg atcacatggt cctgctggag ttcgtgaccg
2641 ccgccgggat cactctcggc atggacgagc tgtacaagta ataggcggac tatgacttag
2701 ttgcgttaca ccctttcttg acaaaaccta acttgcgcag aaaacaagat gagattggca
2761 tggctttatt tgtttttttt gttttgtttt ggtttttttt ttttttttgg cttgactcag
2821 gatttaaaaa ctggaacggt gaaggtgaca gcagtcggtt ggagcgagca tcccccaaag
2881 ttcacaatgt ggccgaggac tttgattgca cattgttgtt tttttaatag tcattccaaa
2941 tatgagatgc gttgttacag gaagtccctt gccatcctaa aagccacccc acttctctct
3001 aaggagaatg gcccagtcct ctcccaagtc cacacagggg aggtgatagc attgctttcg
3061 tgtaaattat gtaatgcaaa atttttttaa tcttcgcctt aatacttttt tattttgttt
3121 tattttgaat gatgagcctt cgtgcccccc cttccccctt ttttgtcccc caacttgaga
3181 tgtatgaagg cttttggtct ccctgggagt gggtggaggc agccagggct tacctgtaca
3241 ctgacttgag accagttgaa taaaagtgca caccttaaaa atgaggccaa gtgtgacttt
3301 gtggtgtggc tgggttgggg gcagcagagg gtgaaccctg caggagggtg aaccctgcaa
3361 aagggtgggg cagtgggggc caacttgtcc ttacccagag tgcaggtgtg tggagatccc
3421 tcctgccttg acattgagca gccttagagg gtgggggagg ctcaggggtc aggtctctgt
3481 tcctgcttat tggggagttc ctggcctggc ccttctatgt ctccccaggt accccagttt
3541 ttctgggttc acccagagtg cagatgcttg aggaggtggg aagggactat ttgggggtgt
3601 ctggctcagg tgccatgcct cactggggct ggttggcacc tgcatttcct gggagt
SA-T2A-EGFP (AAVS-SA-T2A-EGFP-AAVS-PcDNA3.1)
(SEQ ID NO: 37)
      1 gacggatcgg gagatctccc gatcccctat ggtgcactct cagtacaatc tgctctgatg
    61 ccgcatagtt aagccagtat ctgctccctg cttgtgtgtt ggaggtcgct gagtagtgcg
  121 cgagcaaaat ttaagctaca acaaggcaag gcttgaccga caattgcatg aagaatctgc
  181 ttagggttag gcgttttgcg ctgcttcgcg atgtacgggc cagatatacg cgttgacatt
  241 gattattgac tagttattaa tagtaatcaa ttacggggtc attagttcat agcccatata
  301 tggagttccg cgttacataa cttacggtaa atggcccgcc tggctgaccg cccaacgacc
  361 cccgcccatt gacgtcaata atgacgtatg ttcccatagt aacgccaata gggactttcc
  421 attgacgtca atgggtggag tatttacggt aaactgccca cttggcagta catcaagtgt
  481 atcatatgcc aagtacgccc cctattgacg tcaatgacgg taaatggccc gcctggcatt
  541 atgcccagta catgacctta tgggactttc ctacttggca gtacatctac gtattagtca
  601 tcgctattac catggtgatg cggttttggc agtacatcaa tgggcgtgga tagcggtttg
  661 actcacgggg atttccaagt ctccacccca ttgacgtcaa tgggagtttg ttttggcacc
  721 aaaatcaacg ggactttcca aaatgtcgta acaactccgc cccattgacg caaatgggcg
  781 gtaggcgtgt acggtgggag gtctatataa gcagagctct ctggctaact agagaaccca
  841 ctgcttactg gcttatcgaa attaatacga ctcactatag ggagacccaa gctggctagc
  901 gtttaaactt aagcttgggt tctccttctg gggcctgtgc catctctcgt ttcttaggat
  961 ggccttctcc gacggatgtc tcccttgcgt cccgcctccc cttcttgtag gcctgcatca
1021 tcaccgtttt tctggacaac cccaaagtac cccgtctccc tggctttagc cacctctcca
1081 tcctcttgct ttctttgcct ggacaccccg ttctcctgtg gattcgggtc acctctcact
1141 cctttcattt gggcagctcc cctacccccc ttacctctct agtctgtgct agctcttcca
1201 gccccctgtc atggcatctt ccaggggtcc gagagctcag ctagtcttct tcctccaacc
1261 cgggccccta tgtccacttc aggacagcat gtttgctgcc tccagggatc ctgtgtcccc
1321 gagctgggac caccttatat tcccagggcc ggttaatgtg gctctggttc tgggtacttt
1381 tatctgtccc ctccacccca cagtgggggg taccagtcga tccaacatgg cgacttgtcc
1441 catccccggc atgtttaaat atactaatta ttcttgaact aattttaatc aaccgattta
1501 tctctcttcc gcaggtggcg gaggttccgg tggaagcgga ggtagcggcg gatccgaggg
1561 ccgcggcagc ctgctgacct gcggcgatgt ggaggagaac cccgggccca tggtgagcaa
1621 gggcgaggag ctgttcaccg gggtggtgcc catcctggtc gagctggacg gcgacgtaaa
1681 cggccacaag ttcagcgtgt ccggcgaggg cgagggcgat gccacctacg gcaagctgac
1741 cctgaagttc atctgcacca ccggcaagct gcccgtgccc tggcccaccc tcgtgaccac
1801 cctgacctac ggcgtgcagt gcttcagccg ctaccccgac cacatgaagc agcacgactt
1861 cttcaagtcc gccatgcccg aaggctacgt ccaggagcgc accatcttct tcaaggacga
1921 cggcaactac aagacccgcg ccgaggtgaa gttcgagggc gacaccctgg tgaaccgcat
1981 cgagctgaag ggcatcgact tcaaggagga cggcaacatc ctggggcaca agctggagta
2041 caactacaac agccacaacg tctatatcat ggccgacaag cagaagaacg gcatcaaggt
2101 gaacttcaag atccgccaca acatcgagga cggcagcgtg cagctcgccg accactacca
2161 gcagaacacc cccatcggcg acggccccgt gctgctgccc gacaaccact acctgagcac
2221 ccagtccgcc ctgagcaaag accccaacga gaagcgcgat cacatggtcc tgctggagtt
2281 cgtgaccgcc gccgggatca ctctcggcat ggacgagctg tacaagtaag aattcccact
2341 agggacagga ttggtgacag aaaagcccca tccttaggcc tcctccttcc tagtctcctg
2401 atattgggtc taacccccac ctcctgttag gcagattcct tatctggtga cacaccccca
2461 tttcctggag ccatctctct ccttgccaga acctctaagg tttgcttacg atggagccag
2521 agaggatcct gggagggaga gcttggcagg gggtgggagg gaaggggggg atgcgtgacc
2581 tgcccggttc tcagtggcca ccctgcgcta ccctctccca gaacctgagc tgctctgacg
2641 cggctgtctg gtgcgtttca ctgatcctgg tgctgcagct tccttacact tcccaagagg
2701 agaagcagtt tggaaaaaca aaatcagaat aagttggtcc tgagttctaa ctttggctct
2761 tcacctttct agtccccaat ttatattgtt cctccgtgcg tcagttttac ctgtgagata
2821 aggccagtag ccagccccgt cctggcaggg ctgtggtgag gaggggggtg tccgtgtgga
2881 aaactccctt tgtgagaatg gtgcgtccta ggtgttcacc aggtcgtggc cgcctctact
2941 ccctttctct ttctccatcc ttctttcctt aaagagtccc cagtgctatc tgggacatat
3001 tcctccgccc agagcagggt cccgcttccc taaggccctg ctctgggctt ctgggtttga
3061 gtccttggca agcccaggag aggcgctcag gcttccctgt cccccttcct cgtccaccat
3121 ctcatgcccc tggctctcct gccccttccc tacaggggtt cctggctctg ctcttcagac
3181 tgagccccgt ctcgagtcta gagggcccgt ttaaacccgc tgatcagcct cgactgtgcc
3241 ttctagttgc cagccatctg ttgtttgccc ctcccccgtg ccttccttga ccctggaagg
3301 tgccactccc actgtccttt cctaataaaa tgaggaaatt gcatcgcatt gtctgagtag
3361 gtgtcattct attctggggg gtggggtggg gcaggacagc aagggggagg attgggaaga
3421 caatagcagg catgctgggg atgcggtggg ctctatggct tctgaggcgg aaagaaccag
3481 ctggggctct agggggtatc cccacgcgcc ctgtagcggc gcattaagcg cggcgggtgt
3541 ggtggttacg cgcagcgtga ccgctacact tgccagcgcc ctagcgcccg ctcctttcgc
3601 tttcttccct tcctttctcg ccacgttcgc cggctttccc cgtcaagctc taaatcgggg
3661 gctcccttta gggttccgat ttagtgcttt acggcacctc gaccccaaaa aacttgatta
3721 gggtgatggt tcacgtagtg ggccatcgcc ctgatagacg gtttttcgcc ctttgacgtt
3781 ggagtccacg ttctttaata gtggactctt gttccaaact ggaacaacac tcaaccctat
3841 ctcggtctat tcttttgatt tataagggat tttgccgatt tcggcctatt ggttaaaaaa
3901 tgagctgatt taacaaaaat ttaacgcgaa ttaattctgt ggaatgtgtg tcagttaggg
3961 tgtggaaagt ccccaggctc cccagcaggc agaagtatgc aaagcatgca tctcaattag
4021 tcagcaacca ggtgtggaaa gtccccaggc tccccagcag gcagaagtat gcaaagcatg
4081 catctcaatt agtcagcaac catagtcccg cccctaactc cgcccatccc gcccctaact
4141 ccgcccagtt ccgcccattc tccgccccat ggctgactaa ttttttttat ttatgcagag
4201 gccgaggccg cctctgcctc tgagctattc cagaagtagt gaggaggctt ttttggaggc
4261 ctaggctttt gcaaaaagct cccgggagct tgtatatcca ttttcggatc tgatcaagag
4321 acaggatgag gatcgtttcg catgattgaa caagatggat tgcacgcagg ttctccggcc
4381 gcttgggtgg agaggctatt cggctatgac tgggcacaac agacaatcgg ctgctctgat
4441 gccgccgtgt tccggctgtc agcgcagggg cgcccggttc tttttgtcaa gaccgacctg
4501 tccggtgccc tgaatgaact gcaggacgag gcagcgcggc tatcgtggct ggccacgacg
4561 ggcgttcctt gcgcagctgt gctcgacgtt gtcactgaag cgggaaggga ctggctgcta
4621 ttgggcgaag tgccggggca ggatctcctg tcatctcacc ttgctcctgc cgagaaagta
4681 tccatcatgg ctgatgcaat gcggcggctg catacgcttg atccggctac ctgcccattc
4741 gaccaccaag cgaaacatcg catcgagcga gcacgtactc ggatggaagc cggtcttgtc
4801 gatcaggatg atctggacga agagcatcag gggctcgcgc cagccgaact gttcgccagg
4861 ctcaaggcgc gcatgcccga cggcgaggat ctcgtcgtga cccatggcga tgcctgcttg
4921 ccgaatatca tggtggaaaa tggccgcttt tctggattca tcgactgtgg ccggctgggt
4981 gtggcggacc gctatcagga catagcgttg gctacccgtg atattgctga agagcttggc
5041 ggcgaatggg ctgaccgctt cctcgtgctt tacggtatcg ccgctcccga ttcgcagcgc
5101 atcgccttct atcgccttct tgacgagttc ttctgagcgg gactctgggg ttcgaaatga
5161 ccgaccaagc gacgcccaac ctgccatcac gagatttcga ttccaccgcc gccttctatg
5221 aaaggttggg cttcggaatc gttttccggg acgccggctg gatgatcctc cagcgcgggg
5281 atctcatgct ggagttcttc gcccacccca acttgtttat tgcagcttat aatggttaca
5341 aataaagcaa tagcatcaca aatttcacaa ataaagcatt tttttcactg cattctagtt
5401 gtggtttgtc caaactcatc aatgtatctt atcatgtctg tataccgtcg acctctagct
5461 agagcttggc gtaatcatgg tcatagctgt ttcctgtgtg aaattgttat ccgctcacaa
5521 ttccacacaa catacgagcc ggaagcataa agtgtaaagc ctggggtgcc taatgagtga
5581 gctaactcac attaattgcg ttgcgctcac tgcccgcttt ccagtcggga aacctgtcgt
5641 gccagctgca ttaatgaatc ggccaacgcg cggggagagg cggtttgcgt attgggcgct
5701 cttccgcttc ctcgctcact gactcgctgc gctcggtcgt tcggctgcgg cgagcggtat
5761 cagctcactc aaaggcggta atacggttat ccacagaatc aggggataac gcaggaaaga
5821 acatgtgagc aaaaggccag caaaaggcca ggaaccgtaa aaaggccgcg ttgctggcgt
5881 ttttccatag gctccgcccc cctgacgagc atcacaaaaa tcgacgctca agtcagaggt
5941 ggcgaaaccc gacaggacta taaagatacc aggcgtttcc ccctggaagc tccctcgtgc
6001 gctctcctgt tccgaccctg ccgcttaccg gatacctgtc cgcctttctc ccttcgggaa
6061 gcgtggcgct ttctcatagc tcacgctgta ggtatctcag ttcggtgtag gtcgttcgct
6121 ccaagctggg ctgtgtgcac gaaccccccg ttcagcccga ccgctgcgcc ttatccggta
6181 actatcgtct tgagtccaac ccggtaagac acgacttatc gccactggca gcagccactg
6241 gtaacaggat tagcagagcg aggtatgtag gcggtgctac agagttcttg aagtggtggc
6301 ctaactacgg ctacactaga agaacagtat ttggtatctg cgctctgctg aagccagtta
6361 ccttcggaaa aagagttggt agctcttgat ccggcaaaca aaccaccgct ggtagcggtt
6421 tttttgtttg caagcagcag attacgcgca gaaaaaaagg atctcaagaa gatcctttga
6481 tcttttctac ggggtctgac gctcagtgga acgaaaactc acgttaaggg attttggtca
6541 tgagattatc aaaaaggatc ttcacctaga tccttttaaa ttaaaaatga agttttaaat
6601 caatctaaag tatatatgag taaacttggt ctgacagtta ccaatgctta atcagtgagg
6661 cacctatctc agcgatctgt ctatttcgtt catccatagt tgcctgactc cccgtcgtgt
6721 agataactac gatacgggag ggcttaccat ctggccccag tgctgcaatg ataccgcgag
6781 acccacgctc accggctcca gatttatcag caataaacca gccagccgga agggccgagc
6841 gcagaagtgg tcctgcaact ttatccgcct ccatccagtc tattaattgt tgccgggaag
6901 ctagagtaag tagttcgcca gttaatagtt tgcgcaacgt tgttgccatt gctacaggca
6961 tcgtggtgtc acgctcgtcg tttggtatgg cttcattcag ctccggttcc caacgatcaa
7021 ggcgagttac atgatccccc atgttgtgca aaaaagcggt tagctccttc ggtcctccga
7081 tcgttgtcag aagtaagttg gccgcagtgt tatcactcat ggttatggca gcactgcata
7141 attctcttac tgtcatgcca tccgtaagat gcttttctgt gactggtgag tactcaacca
7201 agtcattctg agaatagtgt atgcggcgac cgagttgctc ttgcccggcg tcaatacggg
7261 ataataccgc gccacatagc agaactttaa aagtgctcat cattggaaaa cgttcttcgg
7321 ggcgaaaact ctcaaggatc ttaccgctgt tgagatccag ttcgatgtaa cccactcgtg
7381 cacccaactg atcttcagca tcttttactt tcaccagcgt ttctgggtga gcaaaaacag
7441 gaaggcaaaa tgccgcaaaa aagggaataa gggcgacacg gaaatgttga atactcatac
7501 tcttcctttt tcaatattat tgaagcattt atcagggtta ttgtctcatg agcggataca
7561 tatttgaatg tatttagaaa aataaacaaa taggggttcc gcgcacattt ccccgaaaag
7621 tgccacctga cgtc
sgAAVS1-mCherry plasmid (PUC57-CMV-mCherry-U6-sgRNA)
(SEQ ID NO: 65)
      1 gacgaaaggg cctcgtgata cgcctatttt tataggttaa tgtcatgata ataatggttt
    61 cttagacgtc aggtggcact tttcggggaa atgtgcgcgg aacccctatt tgtttatttt
  121 tctaaataca ttcaaatatg tatccgctca tgagacaata accctgataa atgcttcaat
  181 aatattgaaa aaggaagagt atgagtattc aacatttccg tgtcgccctt attccctttt
  241 ttgcggcatt ttgccttcct gtttttgctc acccagaaac gctggtgaaa gtaaaagatg
  301 ctgaagatca gttgggtgca cgagtgggtt acatcgaact ggatctcaac agcggtaaga
  361 tccttgagag ttttcgcccc gaagaacgtt ttccaatgat gagcactttt aaagttctgc
  421 tatgtggcgc ggtattatcc cgtattgacg ccgggcaaga gcaactcggt cgccgcatac
  481 actattctca gaatgacttg gttgagtact caccagtcac agaaaagcat cttacggatg
  541 gcatgacagt aagagaatta tgcagtgctg ccataaccat gagtgataac actgcggcca
  601 acttacttct gacaacgatc ggaggaccga aggagctaac cgcttttttg cacaacatgg
  661 gggatcatgt aactcgcctt gatcgttggg aaccggagct gaatgaagcc ataccaaacg
  721 acgagcgtga caccacgatg cctgtagcaa tggcaacaac gttgcgcaaa ctattaactg
  781 gcgaactact tactctagct tcccggcaac aattaataga ctggatggag gcggataaag
  841 ttgcaggacc acttctgcgc tcggcccttc cggctggctg gtttattgct gataaatctg
  901 gagccggtga gcgtgggtct cgcggtatca ttgcagcact ggggccagat ggtaagccct
  961 cccgtatcgt agttatctac acgacgggga gtcaggcaac tatggatgaa cgaaatagac
1021 agatcgctga gataggtgcc tcactgatta agcattggta actgtcagac caagtttact
1081 catatatact ttagattgat ttaaaacttc atttttaatt taaaaggatc taggtgaaga
1141 tcctttttga taatctcatg accaaaatcc cttaacgtga gttttcgttc cactgagcgt
1201 cagaccccgt agaaaagatc aaaggatctt cttgagatcc tttttttctg cgcgtaatct
1261 gctgcttgca aacaaaaaaa ccaccgctac cagcggtggt ttgtttgccg gatcaagagc
1321 taccaactct ttttccgaag gtaactggct tcagcagagc gcagatacca aatactgttc
1381 ttctagtgta gccgtagtta ggccaccact tcaagaactc tgtagcaccg cctacatacc
1441 tcgctctgct aatcctgtta ccagtggctg ctgccagtgg cgataagtcg tgtcttaccg
1501 ggttggactc aagacgatag ttaccggata aggcgcagcg gtcgggctga acggggggtt
1561 cgtgcacaca gcccagcttg gagcgaacga cctacaccga actgagatac ctacagcgtg
1621 agctatgaga aagcgccacg cttcccgaag ggagaaaggc ggacaggtat ccggtaagcg
1681 gcagggtcgg aacaggagag cgcacgaggg agcttccagg gggaaacgcc tggtatcttt
1741 atagtcctgt cgggtttcgc cacctctgac ttgagcgtcg atttttgtga tgctcgtcag
1801 gggggcggag cctatggaaa aacgccagca acgcggcctt tttacggttc ctggcctttt
1861 gctggccttt tgctcacatg ttctttcctg cgttatcccc tgattctgtg gataaccgta
1921 ttaccgcctt tgagtgagct gataccgctc gccgcagccg aacgaccgag cgcagcgagt
1981 cagtgagcga ggaagcggaa gagcgcccaa tacgcaaacc gcctctcccc gcgcgttggc
2041 cgattcatta atgcagctgg cacgacaggt ttcccgactg gaaagcgggc agtgagcgca
2101 acgcaattaa tgtgagttag ctcactcatt aggcacccca ggctttacac tttatgcttc
2161 cggctcgtat gttgtgtgga attgtgagcg gataacaatt tcacacagga aacagctatg
2221 accatgatta cgccaagctt gacattgatt attgactagt tattaatagt aatcaattac
2281 ggggtcatta gttcatagcc catatatgga gttccgcgtt acataactta cggtaaatgg
2341 cccgcctggc tgaccgccca acgacccccg cccattgacg tcaataatga cgtatgttcc
2401 catagtaacg ccaataggga ctttccattg acgtcaatgg gtggagtatt tacggtaaac
2461 tgcccacttg gcagtacatc aagtgtatca tatgccaagt ccgcccccta ttgacgtcaa
2521 tgacggtaaa tggcccgcct ggcattatgc ccagtacatg accttacggg actttcctac
2581 ttggcagtac atctacgtat tagtcatcgc tattaccatg gtgatgcggt tttggcagta
2641 caccaatggg cgtggatagc ggtttgactc acggggattt ccaagtctcc accccattga
2701 cgtcaatggg agtttgtttt ggcaccaaaa tcaacgggac tttccaaaat gtcgtaataa
2761 ccccgccccg ttgacgcaaa tgggcggtag gcgtgtacgg tgggaggtct atataagcag
2821 agctggatcc accggtcgcc accatggtga gcaagggcga ggaggataac atggccatca
2881 tcaaggagtt catgcgcttc aaggtgcaca tggagggctc cgtgaacggc cacgagttcg
2941 agatcgaggg cgagggcgag ggccgcccct acgagggcac ccagaccgcc aagctgaagg
3001 tgaccaaggg tggccccctg cccttcgcct gggacatcct gtcccctcag ttcatgtacg
3061 gctccaaggc ctacgtgaag caccccgccg acatccccga ctacttgaag ctgtccttcc
3121 ccgagggctt caagtgggag cgcgtgatga acttcgagga cggcggcgtg gtgaccgtga
3181 cccaggactc ctccctgcag gacggcgagt tcatctacaa ggtgaagctg cgcggcacca
3241 acttcccctc cgacggcccc gtaatgcaga agaagaccat gggctgggag gcctcctccg
3301 agcggatgta ccccgaggac ggcgccctga agggcgagat caagcagagg ctgaagctga
3361 aggacggcgg ccactacgac gctgaggtca agaccaccta caaggccaag aagcccgtgc
3421 agctgcccgg cgcctacaac gtcaacatca agttggacat cacctcccac aacgaggact
3481 acaccatcgt ggaacagtac gaacgcgccg agggccgcca ctccaccggc ggcatggacg
3541 agctgtacaa gtaagtcgac gggcccggga tccgatggaa cactagtgag ggcctatttc
3601 ccatgattcc ttcatatttg catatacgat acaaggctgt tagagagata attggaatta
3661 atttgactgt aaacacaaag atattagtac aaaatacgtg acgtagaaag taataatttc
3721 ttgggtagtt tgcagtttta aaattatgtt ttaaaatgga ctatcatatg cttaccgtaa
3781 cttgaaagta tttcgatttc ttggctttat atatcttgtg gaaaggacga aacaccgggt
3841 cttcgagaag acctgtttta gagctagaaa tagcaagtta aaataaggct agtccgttat
3901 caacttgaaa aagtggcacc gagtcggtgc ttttttgttt tctcgaggga acatctagat
3961 gcattcgcga ggtaccgagc tcgaattcac tggccgtcgt tttacaacgt cgtgactggg
4021 aaaaccctgg cgttacccaa cttaatcgcc ttgcagcaca tccccctttc gccagctggc
4081 gtaatagcga agaggcccgc accgatcgcc cttcccaaca gttgcgcagc ctgaatggcg
4141 aatggcgcct gatgcggtat tttctcctta cgcatctgtg cggtatttca caccgcatat
4201 ggtgcactct cagtacaatc tgctctgatg ccgcatagtt aagccagccc cgacacccgc
4261 caacacccgc tgacgcgccc tgacgggctt gtctgctccc ggcatccgct tacagacaag
4321 ctgtgaccgt ctccgggagc tgcatgtgtc agaggttttc accgtcatca ccgaaacgcg
4381 cga
```

Cell culture and transient transfection: HEK293, HEK293T, HEK293FT and HeLa cell lines were used in this study. Cells were maintained in complete media (DMEM (Invitrogen/Thermofisher) with 10% FBS (Gibco), penicillin (100 U/ml) and streptomycin (100 μg/ml) (Life Technologies/Thermofisher)) in 37° C., 5% CO₂ incubators. Before performing the activation and repression experiments, Cas9-stable expressed cell lines, HEK293-Cas9, HEK293T-Cas9, HEK293FT-Cas9, and HeLa-Cas9 were generated, either by stable integration or by transduction with Cas9 lentivirus (Cas9-Puro or Cas9-Blast), followed by puromycin or blasticidin selection. All the activation and repression experiments were based on Cas9 stable-expression cell lines. The cells were cultured in 24-well plates (Corning) in complete media and transfected with plasmids using Lipofectamine 3000 (Invitrogen) in accordance with the manufacturer's instructions. In brief, 100,000 cells/well were seeded into 24-well plates 12 h before transfection. 600 ng of plasmid encoding dgRNA-MS2:MPH or dgRNA-Com:CK were transfected with 1 μl Lipofectamine 3000 and 1 μl P3000 reagent in Opti-MEM (Invitrogen). Cells were trypsinized and re-seeded into another 24-well plate 24 h after transfection. After 12 h of plating, cells were transfected with a 1:1 mass ratio of sgRNA plasmid and PCR HR donor. 600 ng total plasmid per well was transfected with 1 μl Lipofectamine 3000 and 1 μl P3000 reagent. Puromycin (0.5 g/mL), Zeocin (200 μg/mL), or Blasticidin (5 μg/mL) were added after 24 h of transfection. Media was changed per 24 h with fresh pre-warmed selection media. For Tet-On induction of gene expression, cells were treated 2 days with doxycycline at 1 μg/ml.

Lentivirus production and transduction: Briefly, HEK293FT cells (ThermoFisher) were cultured in DMEM (Invitrogen)+10% FBS (Sigma) media and seeded in 15-cm dishes before transfection. When cell confluency reached 80-90%, the media was replaced by 13 mL pre-warmed OptiMEM (Invitrogen). For transfection of each dish, 20 Fg transfer plasmids, 15 μg psPAX2 (Addgene 12260), 10 μg pMD 2.G (Addgene 12259), and 130 μL PEI were added into 434 μL OptiMEM, briefly vortexed, and incubated at room temperature for 10 min before added to the 13 mL OptiMEM. The 13 mL OptiMEM was replaced with pre-warmed 10% FBS in DMEM. Lentivirus supernatant was harvested 48 h after media change and aliquoted, and stored at −80° C. freezer. For Cas9-Puro or Cas9-Blast transduction, HEK293, HEK293T, HEK293FT, and HeLa cell lines were transduced with Cas9-Puro or Cas9-Blast lentivirus and supplemented with 2 μl of 2 mg/mL polybrene (Millipore) in 6-well plates. The puromycin (0.5 μg/mL) or blasticidin (5 μg/mL) selection was performed for 7 days after lentivirus transduction. For dgCDK1-MS2-MPH lentivirus transduction of HEK293FT-Cas9 cell line, hygromycin (200 μg/mL) selection was performed for 2-3 days.

```
pLY013_pLKO-U6-BsmBI-MS2sgRNA-EFS-HygR-2A-MS2-p65-HSF1.gb
(SEQ ID NO: 38)
      1 ttaatgtagt cttatgcaat actcttgtag tcttgcaaca tggtaacgat gagttagcaa
    61 catgccttac aaggagagaa aaagcaccgt gcatgccgat tggtggaagt aaggtggtac
  121 gatcgtgcct tattaggaag gcaacagacg ggtctgacat ggattggacg aaccactgaa
  181 ttgccgcatt gcagagatat tgtatttaag tgcctagctc gatacataaa cgggtctctc
  241 tggttagacc agatctgagc ctgggagctc tctggctaac tagggaaccc actgcttaag
  301 cctcaataaa gcttgccttg agtgcttcaa gtagtgtgtg cccgtctgtt gtgtgactct
  361 ggtaactaga gatccctcag acccttttag tcagtgtgga aaatctctag cagtggcgcc
  421 cgaacaggga cttgaaagcg aaagggaaac cagaggagct ctctcgacgc aggactcggc
  481 ttgctgaagc gcgcacggca agaggcgagg ggcggcgact ggtgagtacg ccaaaaattt
  541 tgactagcgg aggctagaag gagagagatg ggtgcgagag cgtcagtatt aagcggggga
  601 gaattagatc gcgatgggaa aaaattcggt taaggccagg gggaaagaaa aaatataaat
  661 taaaacatat agtatgggca agcagggagc tagaacgatt cgcagttaat cctggcctgt
  721 tagaaacatc agaaggctgt agacaaatac tgggacagct acaaccatcc cttcagacag
  781 gatcagaaga acttagatca ttatataata cagtagcaac cctctattgt gtgcatcaaa
  841 ggatagagat aaaagacacc aaggaagctt tagacaagat agaggaagag caaaacaaaa
  901 gtaagaccac cgcacagcaa gcggccgctg atcttcagac ctggaggagg agatatgagg
  961 gacaattgga gaagtgaatt atataaatat aaagtagtaa aaattgaacc attaggagta
1021 gcacccacca aggcaaagag aagagtggtg cagagagaaa aaagagcagt gggaatagga
1081 gctttgttcc ttgggttctt gggagcagca ggaagcacta tgggcgcagc gtcaatgacg
1141 ctgacggtac aggccagaca attattgtct ggtatagtgc agcagcagaa caatttgctg
1201 agggctattg aggcgcaaca gcatctgttg caactcacag tctggggcat caagcagctc
1261 caggcaagaa tcctggctgt ggaaagatac ctaaaggatc aacagctcct ggggatttgg
1321 ggttgctctg gaaaactcat ttgcaccact gctgtgcctt ggaatgctag ttggagtaat
1381 aaatctctgg aacagatttg gaatcacacg acctggatgg agtgggacag agaaattaac
1441 aattacacaa gcttaataca ctccttaatt gaagaatcgc aaaaccagca agaaaagaat
1501 gaacaagaat tattggaatt agataaatgg gcaagtttgt ggaattggtt taacataaca
1561 aattggctgt ggtatataaa attattcata atgatagtag gaggcttggt aggtttaaga
1621 atagtttttg ctgtactttc tatagtgaat agagttaggc agggatattc accattatcg
1681 tttcagaccc acctcccaac cccgagggga cccagagagg gcctatttcc catgattcct
1741 tcatatttgc atatacgata caaggctgtt agagagataa ttagaattaa tttgactgta
1801 aacacaaaga tattagtaca aaatacgtga cgtagaaagt aataatttct tgggtagttt
1861 gcagttttaa aattatgttt taaaatggac tatcatatgc ttaccgtaac ttgaaagtat
1921 ttcgatttct tggctttata tatcttgtgg aaaggacgaa acaccggaga cgggataccg
1981 tctctgtttt agagctaggc caacatgagg atcacccatg tctgcagggc ctagcaagtt
2041 aaaataaggc tagtccgtta tcaacttggc caacatgagg atcacccatg tctgcagggc
2101 caagtggcac cgagtcggtg ctttttttgg atccaagctt ggcgtaacta gatcttgaga
2161 caaatggcag tattcatcca caattttaaa agaaaagggg ggattggggg gtacagtgca
2221 ggggaaagaa tagtagacat aatagcaaca gacatacaaa ctaaagaatt acaaaaacaa
2281 attacaaaaa ttcaaaattt tcgggtttat tacagggaca gcagagatcc actttggcgc
2341 cggctcgagg gggcccgggg aattcgctag ctaggtcttg aaaggagtgg gaattggctc
2401 cggtgcccgt cagtgggcag agcgcacatc gcccacagtc cccgagaagt tggggggagg
2461 ggtcggcaat tgatccggtg cctagagaag gtggcgcggg gtaaactggg aaagtgatgt
2521 cgtgtactgg ctccgccttt ttcccgaggg tgggggagaa ccgtatataa gtgcagtagt
2581 cgccgtgaac gttctttttc gcaacgggtt tgccgccaga acacaggacc ggtatgaaaa
2641 agcctgaact caccgctacc tctgtcgaga agtttctgat cgaaaagttc gacagcgtgt
2701 ccgacctgat gcagctctcc gagggcgaag aatctcgggc tttcagcttc gatgtgggag
2761 ggcgtggata tgtcctgcgg gtgaatagct gcgccgatgg tttctacaaa gatcgctatg
2821 tttatcggca ctttgcatcc gccgctctcc ctattcccga agtgcttgac attggggagt
2881 tcagcgagag cctgacctat tgcatctccc gccgtgcaca gggtgtcacc ttgcaagacc
2941 tgcctgaaac cgaactgccc gctgttctcc agcccgtcgc cgaggccatg gatgccatcg
3001 ctgccgccga tcttagccag accagcgggt tcggcccatt cggacctcaa ggaatcggtc
3061 aatacactac atggcgcgat ttcatctgcg ctattgctga tccccatgtg tatcactggc
3121 aaactgtgat ggacgacacc gtcagtgcct ccgtcgccca ggctctcgat gagctgatgc
3181 tttgggccga ggactgcccc gaagtccggc acctcgtgca cgccgatttc ggctccaaca
3241 atgtcctgac cgacaatggc cgcataacag ccgtcattga ctggagcgag gccatgttcg
3301 gggattccca atacgaggtc gccaacatct tcttctggag gccctggttg gcttgtatgg
3361 agcagcagac ccgctacttc gagcggaggc atcccgagct tgcaggatct cctcggctcc
3421 gggcttatat gctccgcatt ggtcttgacc aactctatca gagcttggtt gacggcaatt
3481 tcgatgatgc agcttgggct cagggtcgct gcgacgcaat cgtccggtcc ggagccggga
3541 ctgtcgggcg tacacaaatc gcccgcagaa gcgctgccgt ctggaccgat ggctgtgtgg
3601 aagtgctcgc cgatagtgga aacagacgcc ccagcactcg tcctagggca aagggcagtg
3661 gagagggcag aggaagtctg ctaacatgcg gtgacgtcga ggagaatcct ggcccaatgg
3721 cttcaaactt tactcagttc gtgctcgtgg acaatggtgg gacaggggat gtgacagtgg
3781 ctccttctaa tttcgctaat ggggtggcag agtggatcag ctccaactca cggagccagg
3841 cctacaaggt gacatgcagc gtcaggcagt ctagtgccca gaagagaaag tataccatca
3901 aggtggaggt ccccaaagtg gctacccaga cagtgggcgg agtcgaactg cctgtcgccg
3961 cttggaggtc ctacctgaac atggagctca ctatcccaat tttcgctacc aattctgact
4021 gtgaactcat cgtgaaggca atgcaggggc tcctcaaaga cggtaatcct atcccttccg
4081 ccatcgccgc taactcaggt atctacagcg ctggaggagg tggaagcgga ggaggaggaa
4141 gcggaggagg aggtagcgga cctaagaaaa agaggaaggt ggcggccgct ggatcccctt
4201 cagggcagat cagcaaccag gccctggctc tggcccctag ctccgctcca gtgctggccc
4261 agactatggt gccctctagt gctatggtgc ctctggccca gccacctgct ccagcccctg
4321 tgctgacccc aggaccaccc cagtcactga gcgctccagt gcccaagtct acacaggccg
4381 gcgaggggac tctgagtgaa gctctgctgc acctgcagtt cgacgctgat gaggacctgg
4441 gagctctgct ggggaacagc accgatcccg gagtgttcac agatctggcc tccgtggaca
4501 actctgagtt tcagcagctg ctgaatcagg gcgtgtccat gtctcatagt acagccgaac
4561 caatgctgat ggagtacccc gaagccatta cccggctggt gaccggcagc cagcggcccc
4621 ccgaccccgc tccaactccc ctgggaacca gcggcctgcc taatgggctg tccggagatg
4681 aagacttctc aagcatcgct gatatggact ttagtgccct gctgtcacag atttcctcta
4741 gtgggcaggg aggaggtgga agcggcttca gcgtggacac cagtgccctg ctggacctgt
4801 tcagcccctc ggtgaccgtg cccgacatga gcctgcctga ccttgacagc agcctggcca
4861 gtatccaaga gctcctgtct ccccaggagc cccccaggcc tcccgaggca gagaacagca
4921 gcccggattc agggaagcag ctggtgcact acacagcgca gccgctgttc ctgctggacc
4981 ccggctccgt ggacaccggg agcaacgacc tgccggtgct gtttgagctg ggagagggct
5041 cctacttctc cgaaggggac ggcttcgccg aggaccccac catctccctg ctgacaggct
5101 cggagcctcc caaagccaag gaccccactg tctcctaatg tacaagcgct aataaaagat
5161 ctttattttc attagatctg tgtgttggtt ttttgtgtgg taactctaga cgtgcggtcg
5221 actttaagac caatgactta caaggcagct gtagatctta gccacttttt aaaagaaaag
5281 gggggactgg aagggctaat tcactcccaa cgaagacaag atctgctttt tgcttgtact
5341 gggtctctct ggttagacca gatctgagcc tgggagctct ctggctaact agggaaccca
5401 ctgcttaagc ctcaataaag cttgccttga gtgcttcaag tagtgtgtgc ccgtctgttg
5461 tgtgactctg gtaactagag atccctcaga cccttttagt cagtgtggaa aatctctagc
5521 agtacgtata gtagttcatg tcatcttatt attcagtatt tataacttgc aaagaaatga
5581 atatcagaga gtgagaggaa cttgtttatt gcagcttata atggttacaa ataaagcaat
5641 agcatcacaa atttcacaaa taaagcattt ttttcactgc attctagttg tggtttgtcc
5701 aaactcatca atgtatctta tcatgtctgg ctctagctat cccgccccta actccgccca
5761 tcccgcccct aactccgccc agttccgccc attctccgcc ccatggctga ctaatttttt
5821 ttatttatgc agaggccgag gccgcctcgg cctctgagct attccagaag tagtgaggag
5881 gcttttttgg aggcctaggg acgtacccaa ttcgccctat agtgagtcgt attacgcgcg
5941 ctcactggcc gtcgttttac aacgtcgtga ctgggaaaac cctggcgtta cccaacttaa
6001 tcgccttgca gcacatcccc ctttcgccag ctggcgtaat agcgaagagg cccgcaccga
6061 tcgcccttcc caacagttgc gcagcctgaa tggcgaatgg gacgcgccct gtagcggcgc
6121 attaagcgcg gcgggtgtgg tggttacgcg cagcgtgacc gctacacttg ccagcgccct
6181 agcgcccgct cctttcgctt tcttcccttc ctttctcgcc acgttcgccg gctttccccg
6241 tcaagctcta aatcgggggc tccctttagg gttccgattt agtgctttac ggcacctcga
6301 ccccaaaaaa cttgattagg gtgatggttc acgtagtggg ccatcgccct gatagacggt
6361 ttttcgccct ttgacgttgg agtccacgtt ctttaatagt ggactcttgt tccaaactgg
6421 aacaacactc aaccctatct cggtctattc ttttgattta taagggattt tgccgatttc
6481 ggcctattgg ttaaaaaatg agctgattta acaaaaattt aacgcgaatt ttaacaaaat
6541 attaacgctt acaatttagg tggcactttt cggggaaatg tgcgcggaac ccctatttgt
6601 ttattifict aaatacattc aaatatgtat ccgctcatga gacaataacc ctgataaatg
6661 cttcaataat attgaaaaag gaagagtatg agtattcaac atttccgtgt cgcccttatt
6721 cccttttttg cggcattttg ccttcctgtt tttgctcacc cagaaacgct ggtgaaagta
6781 aaagatgctg aagatcagtt gggtgcacga gtgggttaca tcgaactgga tctcaacagc
6841 ggtaagatcc ttgagagttt tcgccccgaa gaacgttttc caatgatgag cacttttaaa
6901 gttctgctat gtggcgcggt attatcccgt attgacgccg ggcaagagca actcggtcgc
6961 cgcatacact attctcagaa tgacttggtt gagtactcac cagtcacaga aaagcatctt
7021 acggatggca tgacagtaag agaattatgc agtgctgcca taaccatgag tgataacact
7081 gcggccaact tacttctgac aacgatcgga ggaccgaagg agctaaccgc ttttttgcac
7141 aacatggggg atcatgtaac tcgccttgat cgttgggaac cggagctgaa tgaagccata
7201 ccaaacgacg agcgtgacac cacgatgcct gtagcaatgg caacaacgtt gcgcaaacta
7261 ttaactggcg aactacttac tctagcttcc cggcaacaat taatagactg gatggaggcg
7321 gataaagttg caggaccact tctgcgctcg gcccttccgg ctggctggtt tattgctgat
7381 aaatctggag ccggtgagcg tgggtctcgc ggtatcattg cagcactggg gccagatggt
7441 aagccctccc gtatcgtagt tatctacacg acggggagtc aggcaactat ggatgaacga
7501 aatagacaga tcgctgagat aggtgcctca ctgattaagc attggtaact gtcagaccaa
7561 gtttactcat atatacttta gattgattta aaacttcatt tttaatttaa aaggatctag
7621 gtgaagatcc tttttgataa tctcatgacc aaaatccctt aacgtgagtt ttcgttccac
7681 tgagcgtcag accccgtaga aaagatcaaa ggatcttctt gagatccttt ttttctgcgc
7741 gtaatctgct gcttgcaaac aaaaaaacca ccgctaccag cggtggtttg tttgccggat
7801 caagagctac caactctttt tccgaaggta actggcttca gcagagcgca gataccaaat
7861 actgttcttc tagtgtagcc gtagttaggc caccacttca agaactctgt agcaccgcct
7921 acatacctcg ctctgctaat cctgttacca gtggctgctg ccagtggcga taagtcgtgt
7981 cttaccgggt tggactcaag acgatagtta ccggataagg cgcagcggtc gggctgaacg
8041 gggggttcgt gcacacagcc cagcttggag cgaacgacct acaccgaact gagataccta
8101 cagcgtgagc tatgagaaag cgccacgctt cccgaaggga gaaaggcgga caggtatccg
8161 gtaagcggca gggtcggaac aggagagcgc acgagggagc ttccaggggg aaacgcctgg
8221 tatctttata gtcctgtcgg gtttcgccac ctctgacttg agcgtcgatt tttgtgatgc
8281 tcgtcagggg ggcggagcct atggaaaaac gccagcaacg cggccttttt acggttcctg
8341 gccttttgct ggccttttgc tcacatgttc tttcctgcgt tatcccctga ttctgtggat
8401 aaccgtatta ccgcctttga gtgagctgat accgctcgcc gcagccgaac gaccgagcgc
8461 agcgagtcag tgagcgagga agcggaagag cgcccaatac gcaaaccgcc tctccccgcg
8521 cgttggccga ttcattaatg cagctggcac gacaggtttc ccgactggaa agcgggcagt
8581 gagcgcaacg caattaatgt gagttagctc actcattagg caccccaggc tttacacttt
8641 atgcttccgg ctcgtatgtt gtgtggaatt gtgagcggat aacaatttca cacaggaaac
8701 agctatgacc atgattacgc caagcgcgca attaaccctc actaaaggga acaaaagctg
8761 gagctgcaag c
```

RT-qPCR: Cells were collected and lysed using TRlzol (Invitrogen) after 48 h of drug treatment. Total RNA was isolated using RNAiso Plus (Takara). cDNA synthesis was performed using the Advantage RT-for-PCR kit (Takara). RNA levels were quantified by qPCR using SYBR Fast qPCR Mix (Takara) in 20 μl reactions. qPCR was carried out using the CFX96 Touch Real-Time PCR Detection System (Bio-Rad). Melt curves were used to confirm the specificity of primers. mRNA relative expression levels were normalized to GAPDH expression by the ΔΔCt method.

TABLE 2
Primers used for qRT-PCR

| Target | | | |
|---|---|---|---|
| gene | Name | Sequence (5′>3′) | SEQ ID NO |
| GFP | GFP-qF | TGACCTACGGCGTGCAGTGCTT | SEQ ID NO: 39 |
| | GFP-qR | CCTCGAACTTCACCTCGGCGC | SEQ ID NO: 40 |
| GADPH | GADPH-qF | TTTGGTCGTATTGGGCGCCTGG | SEQ ID NO: 41 |
| | GADPH-qR | CTCAGCCTTGACGGTGCCATGG | SEQ ID NO: 42 |
| ASCL1 | ASCL1-qF | GAGGAGCAGGAGCTTCTCGACT | SEQ ID NO: 43 |
| | ASCL1-qR | AACGCCACTGACAAGAAAGCAC | SEQ ID NO: 44 |
| HBG1 | HBG1-qF | GGCTACTATCACAAGCCTGTGG | SEQ ID NO: 45 |
| | HBG1-qR | TTGCCCATGATGGCAGAGGCA | SEQ ID NO: 46 |
| CDK1 | CDK1-qF | CTACAGGTCAAGTGGTAGCCATG | SEQ ID NO: 47 |
| | CDK1-qR | CTGGAATCCTGCATAAGCACATCC | SEQ ID NO: 48 |
| CtIP | CtIP-qF | CAACAGCTGAGGGAACAGCAG | SEQ ID NO: 49 |
| | CtIP-qR | AGTTTAAGATCCTGCTGCCGG | SEQ ID NO: 50 |
| Ligase | LIG4-qF | GGTAAAGGATCACGGGGTGG | SEQ ID NO: 51 |
| IV | LIG4-qR | GCTGCTTGGTGGAGCTTTTC | SEQ ID NO: 52 |
| KU70 | KU70-qF | GGCTGTGGTGTTCTATGGTACCG | SEQ ID NO: 53 |
| | KU70-qR | CCGTGGCCCATCATGTCTTGGA | SEQ ID NO: 54 |
| KU80 | KU80-qF | GTTGTGCTGTGTATGGACGTGG | SEQ ID NO: 55 |
| | KU80-qR | GTGCCATCAGTACCAAACAGGAC | SEQ ID NO: 56 |

Confocal fluorescence imaging: Before performing confocal fluorescence imaging, transfected cells were trypsinized and re-seeded on glass cover slips overnight. After aspirating the medium, cells were treated with 4% formaldehyde/PBS for 15 min for fixation, where their nuclei were stained with DAPI (CST) in PBS. EGFP or mCherry fluorescence was visualized by a confocal microscope (Zeiss LSM 800). Confocal data were analyzed using Image J software (NIH, Bethesda, Md., USA).

Flow cytometry analysis: Flow cytometric (or FACS) assays were used to evaluate the percentage of EGFP- or mCherry-positive cells. Briefly, HEK293-Cas9, HEK293T-Cas9, HEK293FT-Cas9 and HeLa-Cas9 cells were transfected with sgRNA plasmid and HR donor, then cultured for 72 h. The cells were digested by Trypsin without EDTA, followed by briefly centrifugation and resuspension in PBS, then the cell density was determined and diluted to 1×10⁶ cell/mL. Finally, these samples were analyzed using a BD Fortessa or BD FACSAria flow cytometer within one hour.

Genomic DNA isolation and DNA sequencing: The transfected cells were lysed and gDNA was extracted using the DNeasy Tissue Kit (Qiagen) following the manufacturer's instruction. For HDR-positive event identification, PCR was performed using PrimeSTAR HS DNA Polymerase (Takara) with sequence-specific primers (Table 3) using the condition: 95° C. for 4 min; 35 cycles of 95° C. for 20 s, 60° C. for 30 s, 72° C. for 1 min; 72° C. for 2 min for the final extension. PCR products were run on 1.5% agarose gel (Biowest). The specific DNA bands were recovered using AxyPrep DNA Gel Extraction Kit (Axygen). Purified PCR products were cloned into the pMD-19 T vector (Takara) according to the standard manufacturer's instructions or directly sequenced by specific primers. Plasmid mini-preparations were performed using the AxyPrep Plasmid Miniprep Kit (Axygen), and midi-preperations were performed using QIAGEN Plasmid Plus Midi Kit (Qiagen). All sequencing confirmations were carried out using Sanger sequencing.

TABLE 3
Primers for PCR amplification
of sgRNA target region

| Target | | | |
|---|---|---|---|
| gene | Name | Sequence (5′>3′) | SEQ ID NO |
| EGFP | F | AGATCTATGGTGAGCAAGG | SEQ ID NO: 57 |
| | | GCGAGGA | |
| | R | GAATTCTTACTTGTACAGC | SEQ ID NO: 58 |
| | | TCGTCCATG | |
| AAVS1 | Primer-F | GGGTCACCTCTACGGCTGG | SEQ ID NO: 59 |
| | Primer-R | CGAATTCTTACTTGTACAG | SEQ ID NO: 60 |
| | | CTCGTCCA | |

TABLE 4
Target sequences of sgRNAs

| Target | | | |
|---|---|---|---|
| gene | Name | Sequence (5′>3′) | SEQ ID NO |
| Venus | sgVenus | GAGCAGCGTCTTCGAGAGTG | SEQ ID NO: 61 |
| AAVS1 | sgAAVS1-1 | CACCCCACAGTGGGGCCACT | SEQ ID NO: 62 |
| AAVS1 | sgAAVS1-2 | TGTCCCTAGTGGCCCCACTG | SEQ ID NO: 63 |
| ACTB | sgACTB | CCACCGCAAATGCTTCTAGG | SEQ ID NO: 64 |

Cell cycle analysis: Cells were harvested after CRISPR/dgRNAs activation or/and repression for 72 h, and single cell suspensions prepared in PBS with 0.1% BSA. Cells were washed and spun at 400×g for 5 min, resuspended with precooled 70% ethanol, and fixed at 4° C. overnight. Cells were washed in PBS, spun at 500×g for 5 min, resuspended in 500 L PBS containing 50 μg/mL Propidium Iodide (PI), 100 μg/mL RNase, and 0.2% Triton X-100, and incubated at 4° C. for 30 min. Before flow cytometry analysis, cells were passed through a 40 μm cell strainer to remove cell aggregates.

CCK-8 assays: Cell viability was measured using a Cell Counting Kit-8 (CCK-8) assay (Dojindo; CK04). The transfected cells (24 h after transfection) were seeded in a 96-well plate at a density of 2.5-5×10³ cells. Cells were incubated for 1 h with 110 μL complete DMEM media with 10 μL CCK-8 reagent for 24 h. Cell viability detection was performed by measuring the optical absorbance at 450 nm using a multimode reader (Beckman Coulter; DTX880).

Sample size determination: No specific methods were used to predetermine sample size. Experiments were repeated 3 times unless otherwise noted.

Blinding statement: Investigators were not blinded for data collection or analysis. Most experiments were repeated at least 3 times to ensure reproducibility.

The results of the experiments are now described.

### Example 1

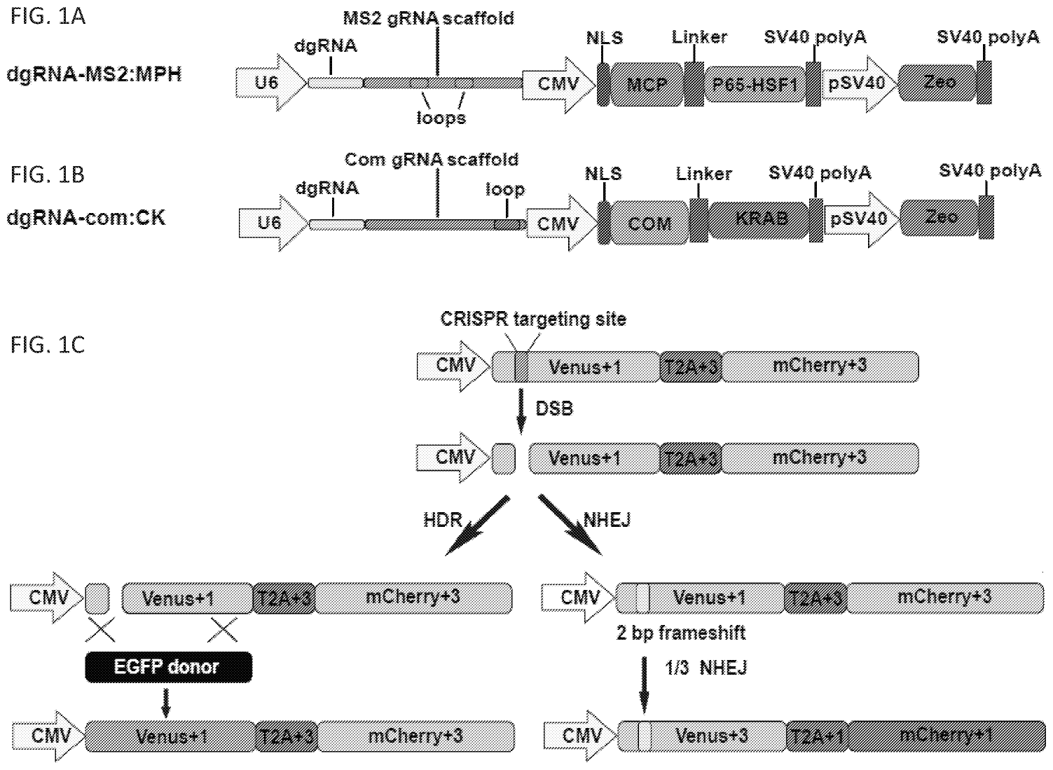

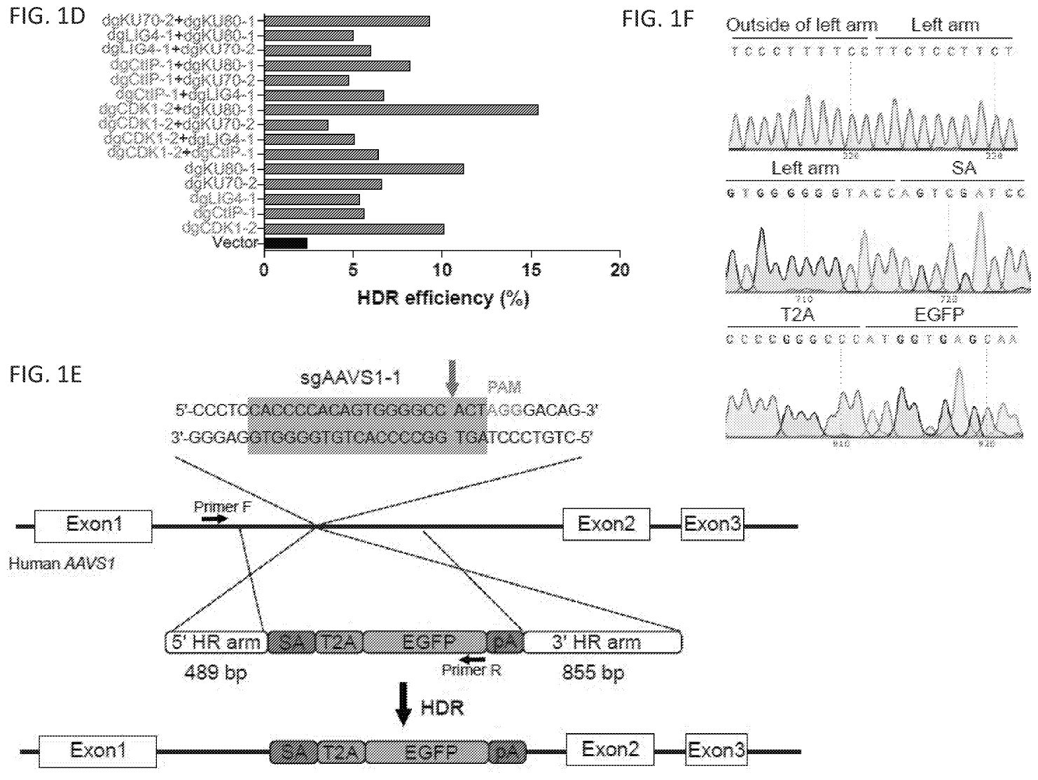

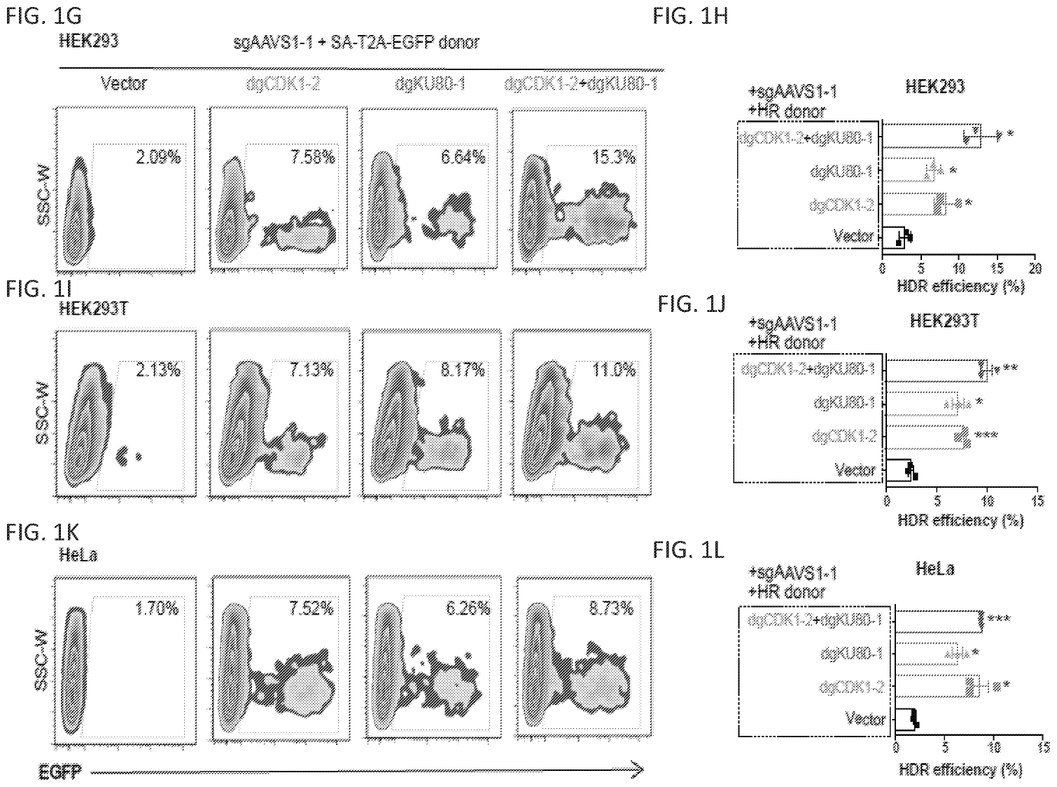

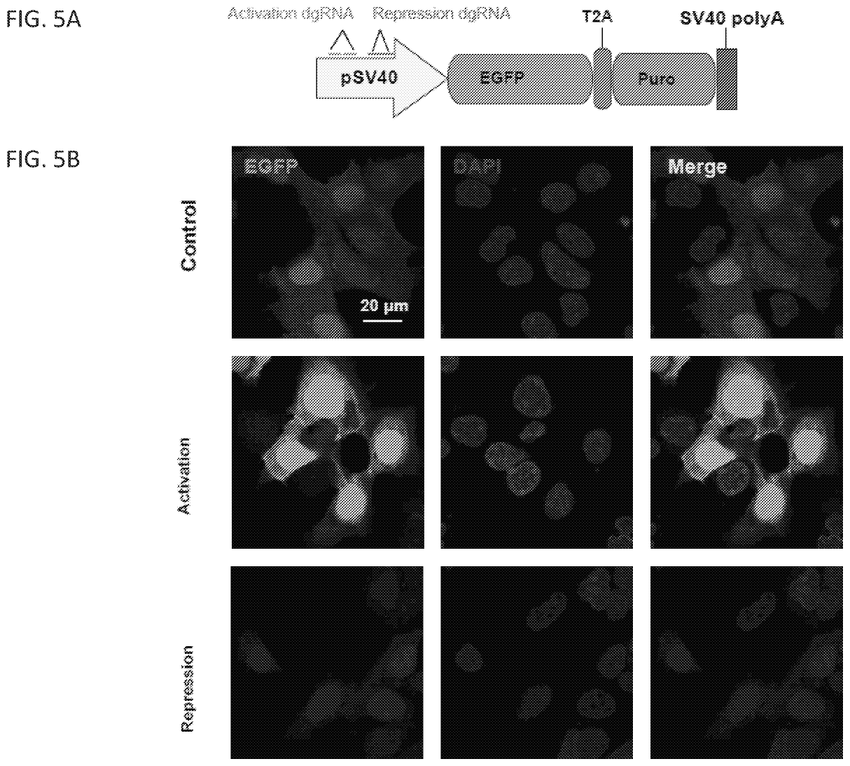

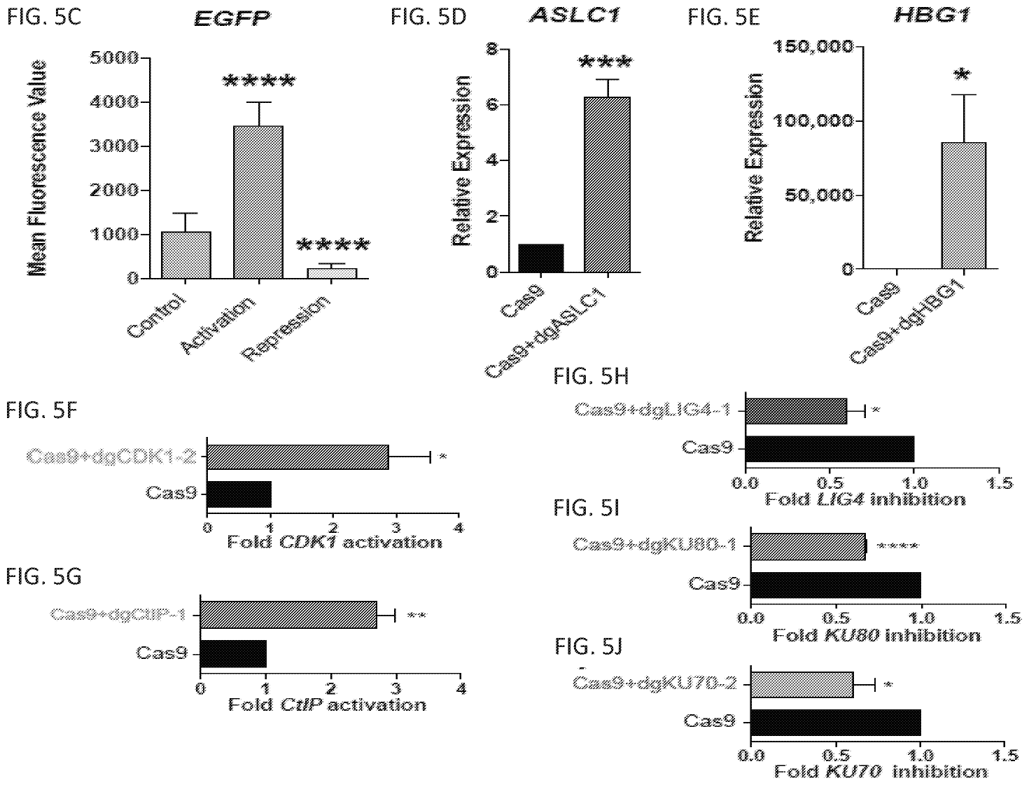

To enhance HDR efficiency of CRISPR-mediated gene editing with clean genetic approaches that avoid the potential side effects from chemical compounds, a method was developed that tunes the expression of DNA damage repair pathway components by dgRNA/active Cas9 mediated CRISPRa and CRISPRi (CRISPRa/i). A Com binding loop was constructed into a dgRNA scaffold for recruiting the Com-KRAB (CK) fusion domain to repress NHEJ-related genes (FIG. 1B). MS2 binding loops were introduced into a into a dgRNA scaffold for recruiting a MCP-P65-HSF1 (MPH) fusion domain to activate HDR-related genes (FIG. 1A). These two constructs were first tested using an EGFP reporter system, and then two endogenous genes. The results showed robust activation and repression of both exogenous reporter genes and endogenous genes, where the EGFP's mRNA level was significantly upregulated by dgGFP-MS2:MPH and repressed by dgGFP-Com:CK (FIG. 5A-5C). The transcriptional level of ASCL1 and HBG1 were dramatically upregulated by dgRNA-MS2:MPH systems with gene-specific dgRNAs (FIG. 5D-5E). Based on the robust functions of dgRNA-MS2:MPH and dgRNA-Com:CK, the activation and repression of several key HDR and NHEJ genes, respectively, were programmed. The results showed that transcript levels of CDK1, which promotes efficient end resection by phosphorylating DSB resection nuclease and CtIP, an enzyme that promotes resection of DNA ends to single-stranded DNA (ssDNA), which is essential for HR, were upregulated by nearly 3-fold (FIGS. 5F-5G). LIG4, KU70 and KU80 transcript levels were reduced by 40-50% (FIGS. 5H-5J).

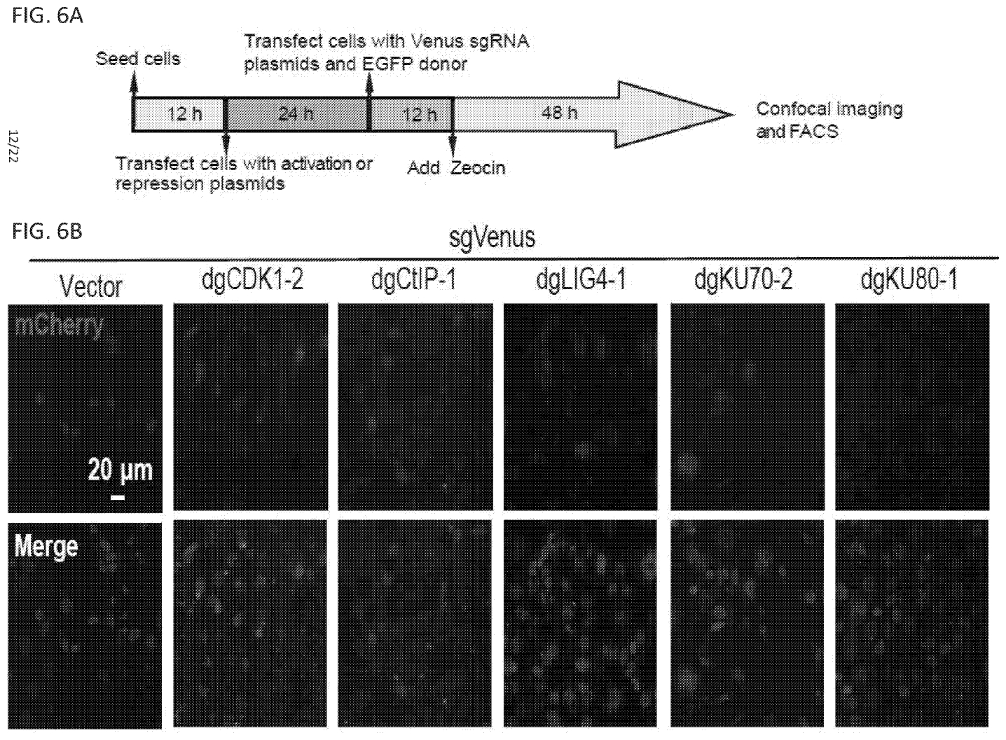

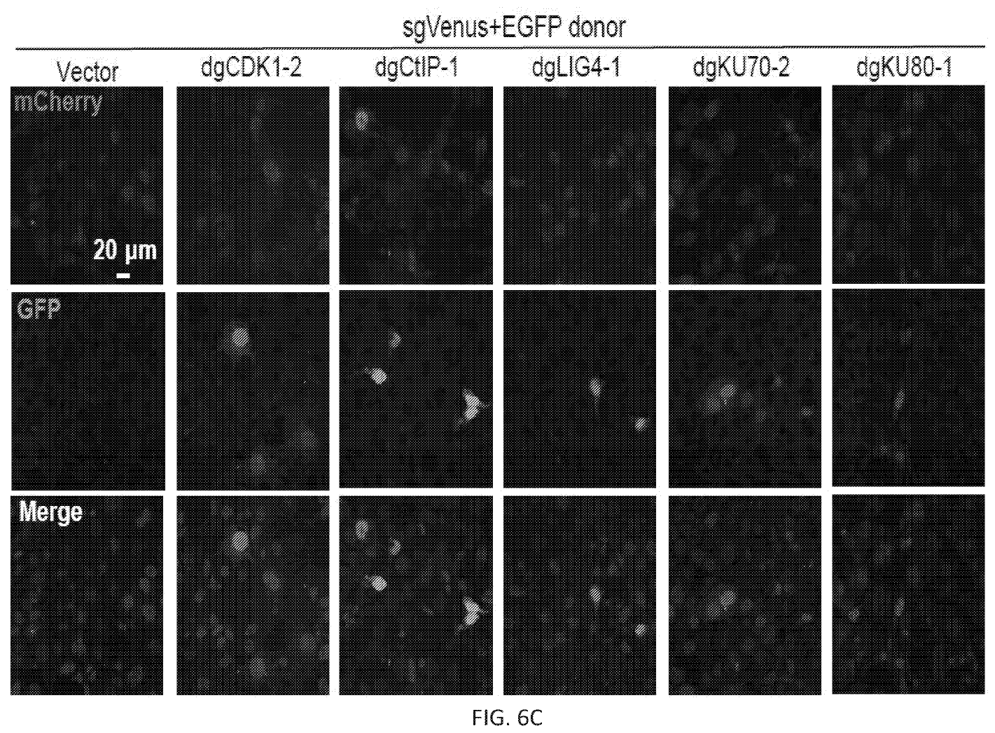

Next, it was determined whether CDK1 and CtIP activation or LIG4, KU70 and KU80 inhibition could enhance HDR frequency for CRISPR-mediated precise gene editing. To quantitatively determine the HDR and NHEJ outcome, a Traffic Light Reporter (TLR) stable expression HEK293 cell line that also expresses Cas9 (HEK293-Cas9-TLR) was generated (FIG. 1C). The TLR included a nonfunctional green fluorescent reporter in which codons 53-63 were disrupted (broken frame Venus, bf-Venus), driven by a CMV promoter. In addition, a self-cleaving peptide T2A and a red fluorescent reporter with a 2 bp frameshift (fs-mCherry) were cloned closely adjacent to the bf-Venus (FIG. 1C). With an sgRNA targeting the 5′ region of the bf-Venus, Cas9 induces DSBs, which can subsequently be repaired by two major DNA repair pathways, NHEJ or HDR. NHEJ causes indels shifting the coding frame of the T2A-mCherry. Approximately ⅓ of the mutagenic NHEJ events generated in-frame functional mCherry that could be detected in cells (FIG. 6B). However, if an intact EGFP HDR donor was provided during DSB repair, the bf-Venus would be corrected in a precise manner that leaves the succeeding fs-mCherry out of frame (FIG. 6C). Thus, this TRL reporter allowed accurate quantification of HDR and NHEJ events.

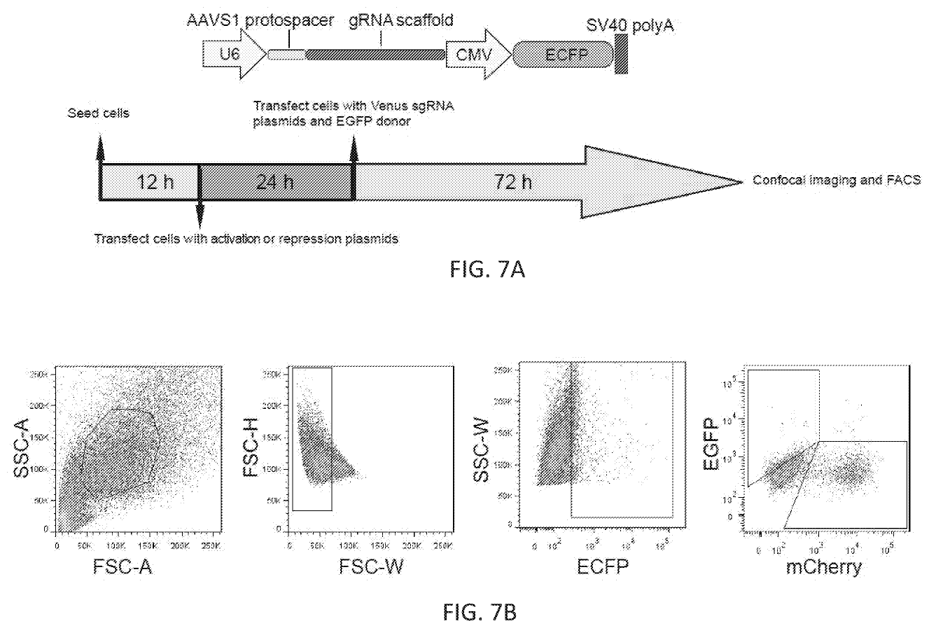

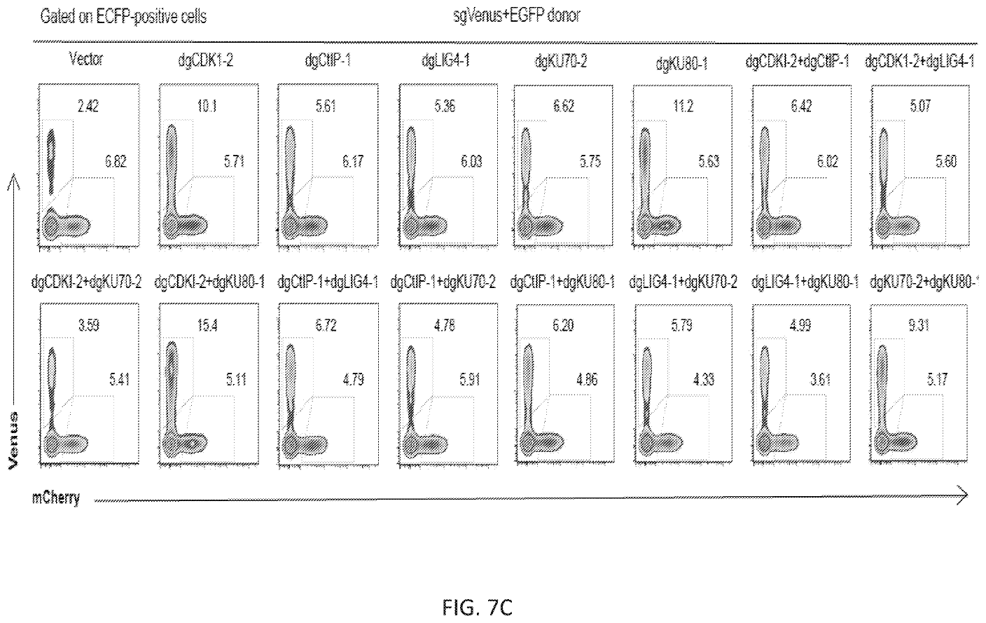

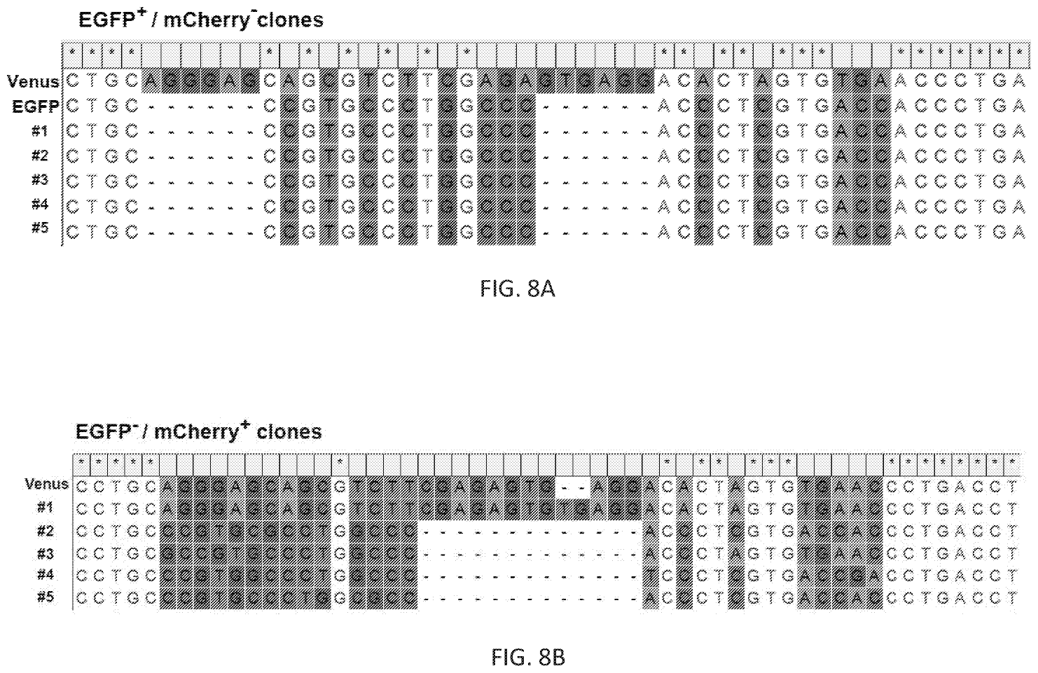

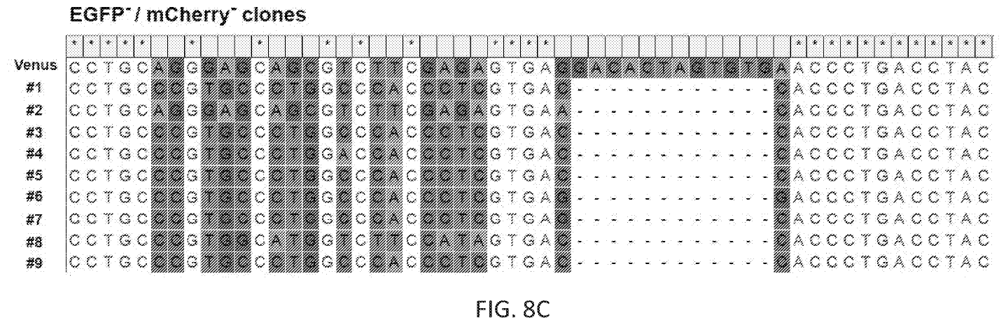

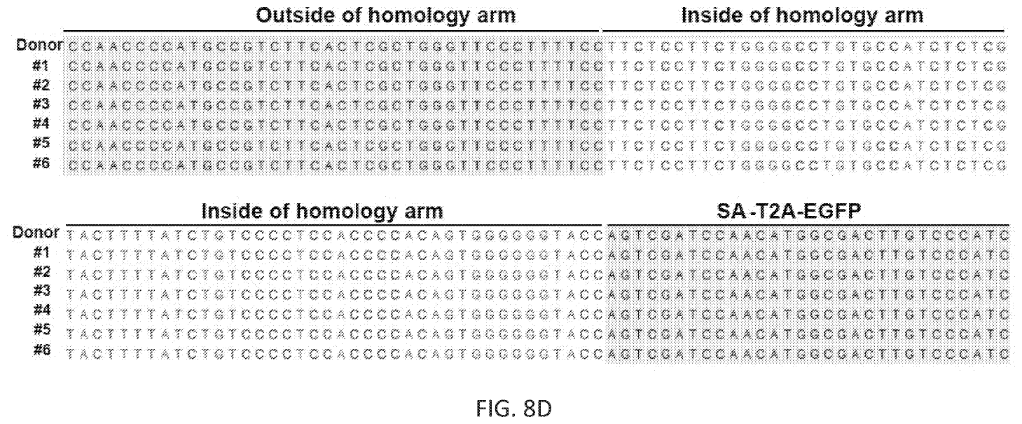

Using this TLR reporter, the HEK293-Cas9-TLR cell line was transfected with dgRNA-Com:CK and/or dgRNA-MS2:MPH plasmids targeting CDK1, CtIP, LIG4, KU70 and KU80 to modulate the expression of these factors. Twenty-four hours later, cells were co-transfected with PCR EGFP HDR template and sgVenus-ECFP expression plasmid (SEQ ID NO: 35) (FIG. 7A). ECFP⁺ cells were gated by FACS after 48 h of transfection (FIG. 7B), and the frequency of EGFP⁺ and mCherry⁺ cells were determined (FIG. 7C). In the vector group, 2.42% EGFP⁺ and 6.82% mCherry⁺ cells were observed, which represented HDR- and NHEJ-positive events, respectively (FIG. 7C). In contrast, the percentage of EGFP⁺ cells was dramatically increased after activating HDR related genes by dgRNA-MS2:MPH, or repressing NHEJ related genes by dgRNA-Com:CK, for most genes or combinations tested (FIG. 1D; FIG. 7C). Particularly, in the group of dgCDK1-2:MS2-MPH (dgCDK1-2)+dgKU80-1:Com-CK (dgKU80-1), 15.4% EGFP-positive cells were observed (FIG. 1D; FIG. 7C). To confirm that the DSBs were repaired through HDR or NHEJ pathways, EGFP⁺/mCherry⁻, EGFP⁻/mCherry⁺ and EGFP⁻/mCherry⁻ cells were cloned and the TLR sgRNA targeting sites were sequenced. It was observed that in EGFP⁺/mCherry⁻ clones, the bf-Venus gene was precisely repaired by the EGFP HDR donor without indels, whereas various indels were found in both EGFP⁻/mCherry⁺ and EGFP⁻/mCherry⁻ clones (FIGS. 8A-8C), confirming the HDR and NHEJ events at the genomic DNA (gDNA) level. Thus, with the robust TLR system, modulating HDR factors, NHEJ factors, or their combinations significantly enhanced HDR efficiency, where both programming HDR/NHEJ by CRISPRa/i and Cas9-mediated gene editing were achieved simultaneously with a single Cas9 transgene.

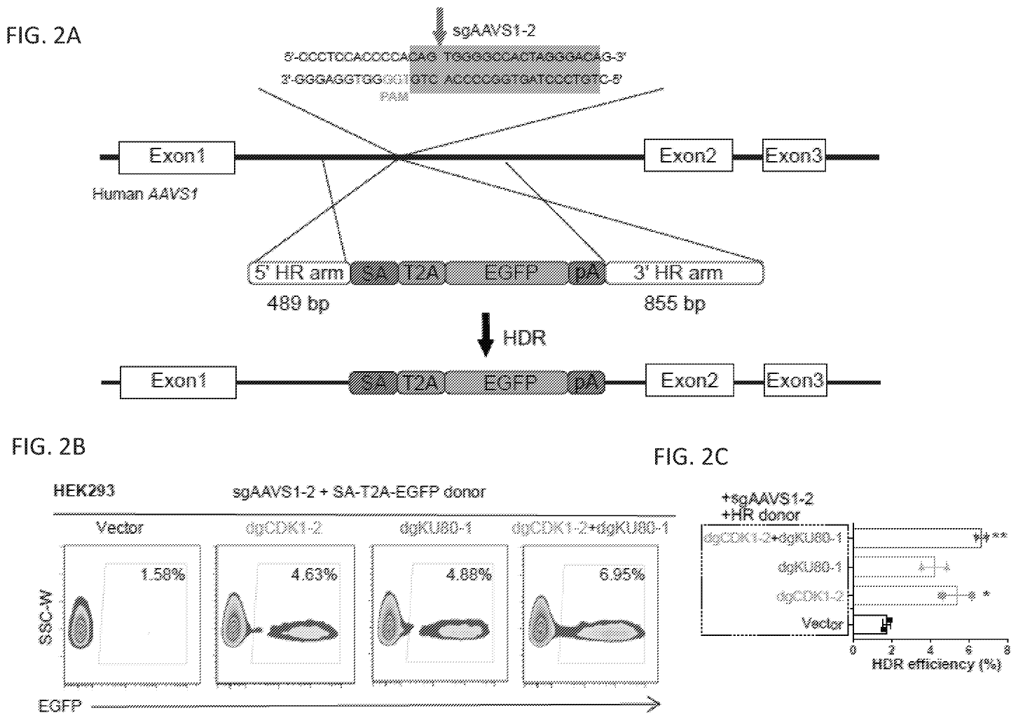

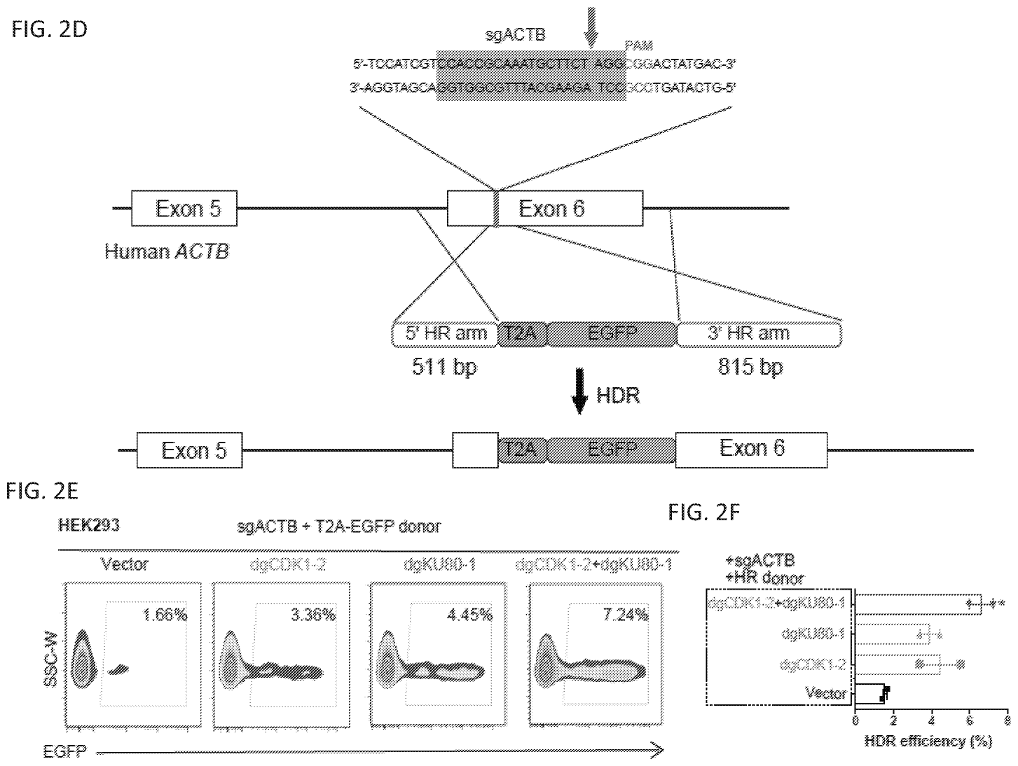

The dgCDK1-2+dgKU80-1 combination had the highest enhancement of HDR efficiency among all tested groups/programs as revealed by the TLR experiment. The effect of this system on CRISPR-mediated gene editing was tested on an endogenous genomic locus by measuring the precise integration of an HDR donor expression cassette, SA-T2A-EGFP (SEQ ID NO: 37; AAVS-SA-T2A-EGFP-AAVS-PcDNA3.1), into the first intron of the canonical AAVS1 locus upon Cas9/sgRNA induced double stranded break (FIG. 1E). The SA-T2A-GFP was flanked by an AAVS1 left homology arm (489 bp) and a right homology arm (855 bp), where EGFP could only be expressed when the SA-T2A-EGFP was precisely recombined into the target site (FIG. 1E). dgRNA-Com:CK and/or dgRNA-MS2:MPH constructs targeting CDK1 and KU80 genes were transfected into the HEK293-Cas9 cell line. Twenty-four hours later, these cells were co-transfected with SA-T2A-EGFP HDR donor template and an sgAAVS1-mCherry plasmid (SEQ ID NO: 65) and then analyzed by FACS 48 h after transfection. Compared to the baseline 2.09% GFP⁺ cells in the mCherry⁺ population in the vector group, the fraction of GFP⁺ cells from dgCDK1-2, dgKU80-1 and dgCDK1-2+dgKU80-1 groups were significantly increased to 7.58%, 6.64% and 15.3%, respectively (FIGS. 1G-1H). Quantitative results showed that HDR efficiency was enhanced over 3 fold with single factor programming and over 7 fold with dual programming on the endogenous AAVS1 locus (FIGS. 1G-1H). Results were confirmed with two additional cell lines, with up to 5 fold HDR enhancement in HEK293T cells and 5 fold in HeLa cells (FIGS. 1I-1L). Another sgRNA was designed for AAVS1 targeting using the same HDR template (FIG. 2A)(SEQ ID NO: 34). The results showed that HDR can also be significantly improved using this sgRNA (FIGS. 2B-2C). In addition, another gene locus, ACTB, was tested. Activation of CDK1 and repression of KU80 significantly enhanced HDR up to 4-5 fold (FIGS. 2D-2F). Results from all cell lines and loci showed that HDR efficiency enhancement was most dramatic in the dgCDK1-2+dgKU80-1 combination group. The endogenous AAVS1 locus was amplified, cloned, and sequenced, confirming the precise integration of SA-T2A-EGFP into the anticipated target site (FIG. 1F, FIG. 8D). Thus, in concordance with the exogenous TLR results, an enhanced efficacy of precise gene targeting via HDR in the native mammalian genome was demonstrated.

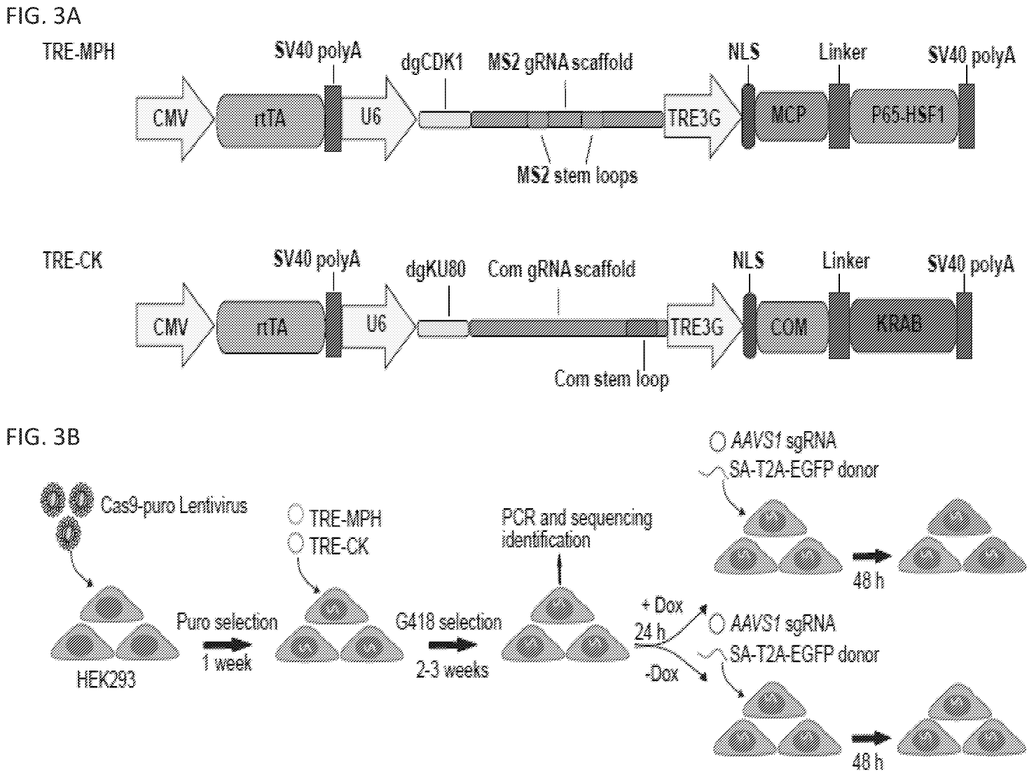

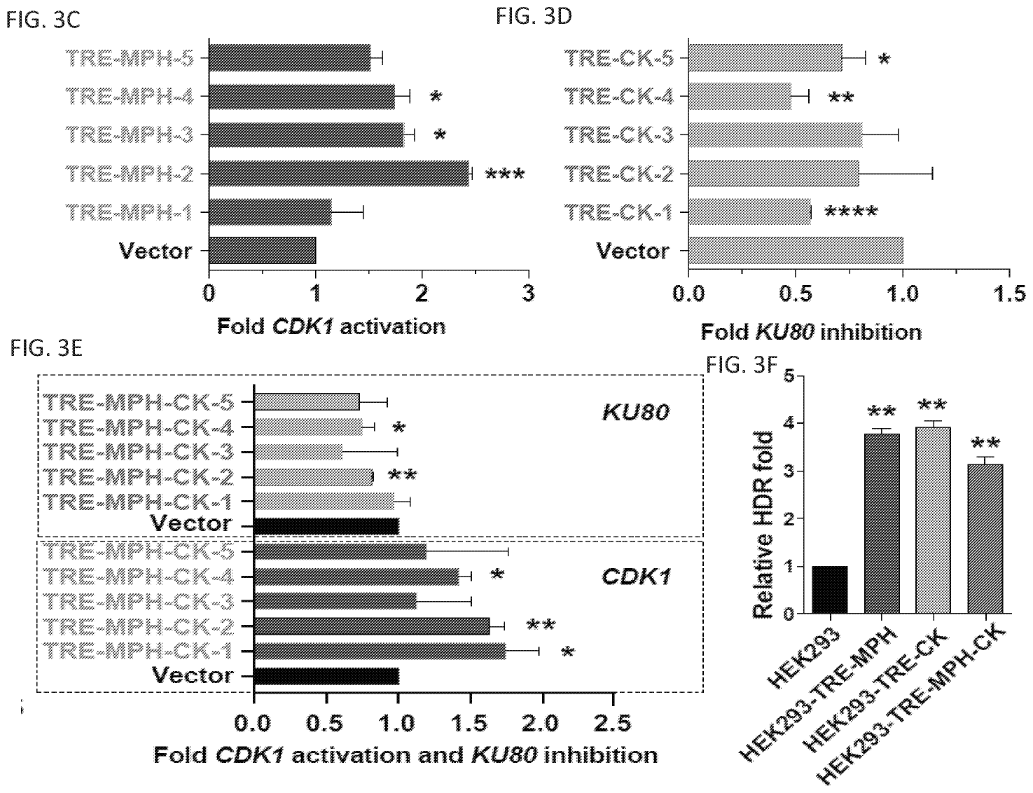

To further improve the programmability, the approach was adapted to additional conditional-expression modules and viral packaging systems. To reduce potential side effects from constitutive activation of CDK1 or deficiency of KU80, a Tet-On system inducible by doxycycline (Dox) was utilized to control the expression of CRISPRa and CRISPRi effectors, MPH and CK, respectively. Two vectors, TRE-MPH and TRE-CK, were constructed (FIG. 3A). Both vectors contain a CMV-rtTA expression cassette. When cells are treated with Dox, the rtTA protein specifically binds to the TRE3G promoter and thereby initiates the transcription of MPH or CK downstream (FIG. 3A), which is reversibly turned off upon Dox removal. These plasmids were transfected into HEK293-Cas9 individually and in combination. G418 selection and cell cloning followed to obtain TRE-MPH, TRE-CK, and TRE-MPH-CK cell lines (FIG. 3B). By qRT-PCR, it was determined that CDK1 and KU80 were significantly activated or repressed, respectively, in a select set of stable cell lines (FIG. 3C-3D). TRE-MPH-2 and TRE-CK-4 were chosen based on their best potency of Dox-induced CDK1 activation and KU80 repression for the subsequent endogenous HDR experiments.

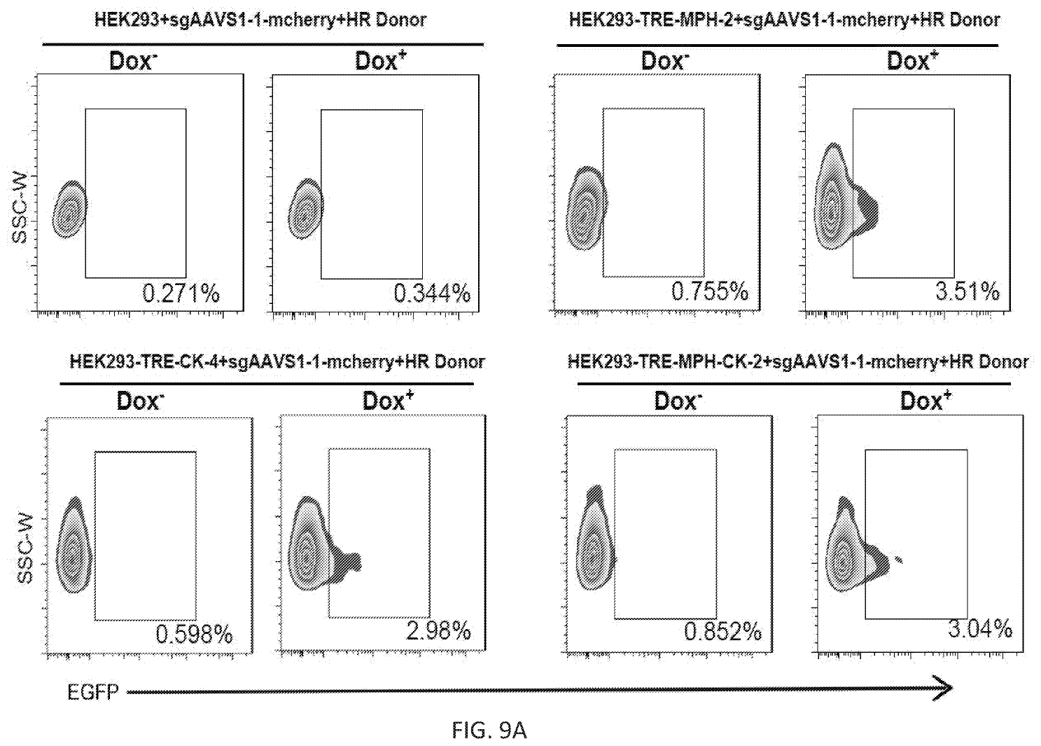

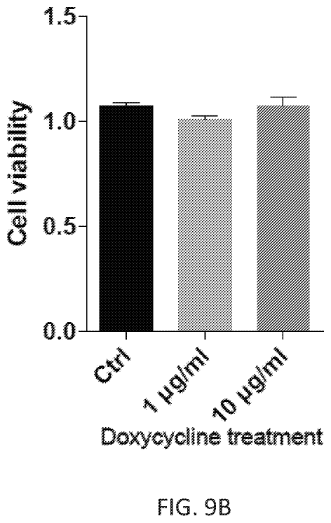

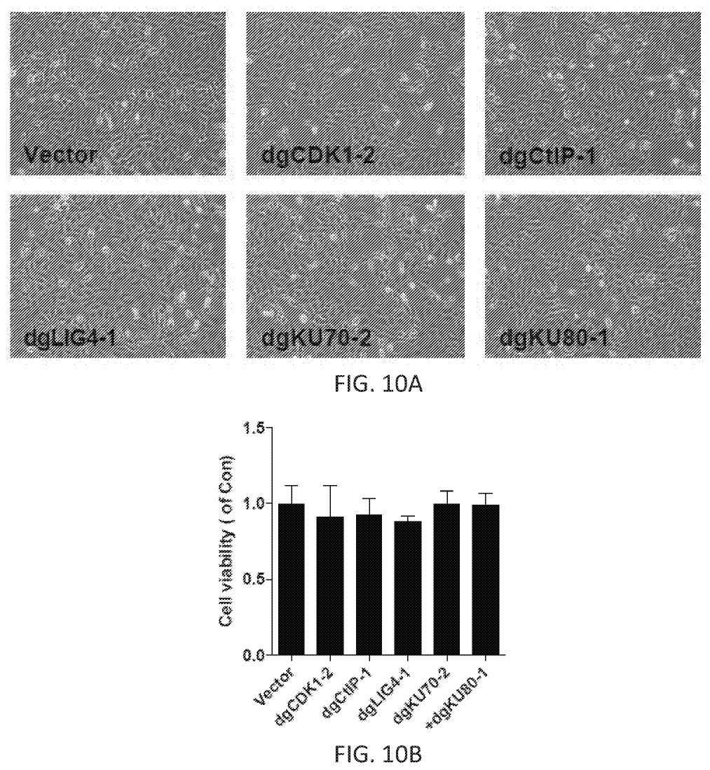

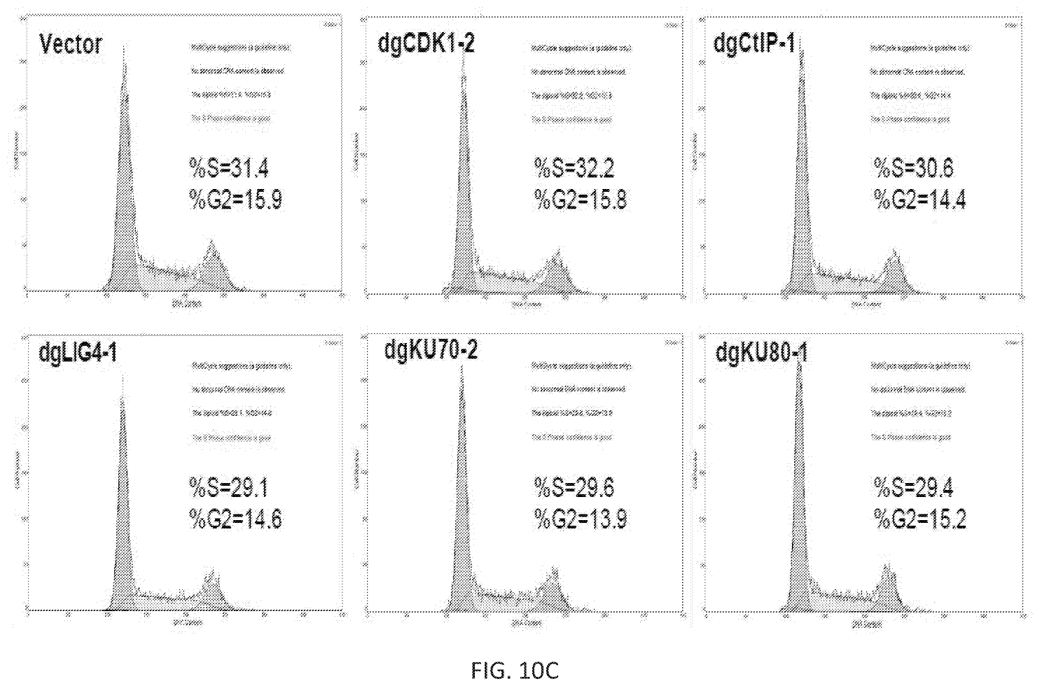

Three different cell lines were treated with Dox for 24 h, then the SA-T2A-EGFP HDR donor for AAVS1 locus and sgAAVS1-mCherry plasmid were co-transfected. After 48 h of transfection, EGFP⁺ cells in mCherry⁺ population were quantified by FACS. Upon Dox treatment, the percentages of EGFP⁺ cells significantly increased in all three groups as compared to control (FIG. 9A), and without any side effects for Dox (FIG. 9B). Albeit, a similar 4-fold enhancement was observed, possibly due to the capacity of Dox-inducible gene expression. Although the transcriptional levels of CDK1 activation or KU80 repression can vary between clones, the clones with significant CDK1 activation and/or KU80 repression showed increased HDR efficiency. These data demonstrate that the CRISPRa/i DNA repair programming can be used in conjunction with an inducible expression system to allow further control of HDR enhancement.

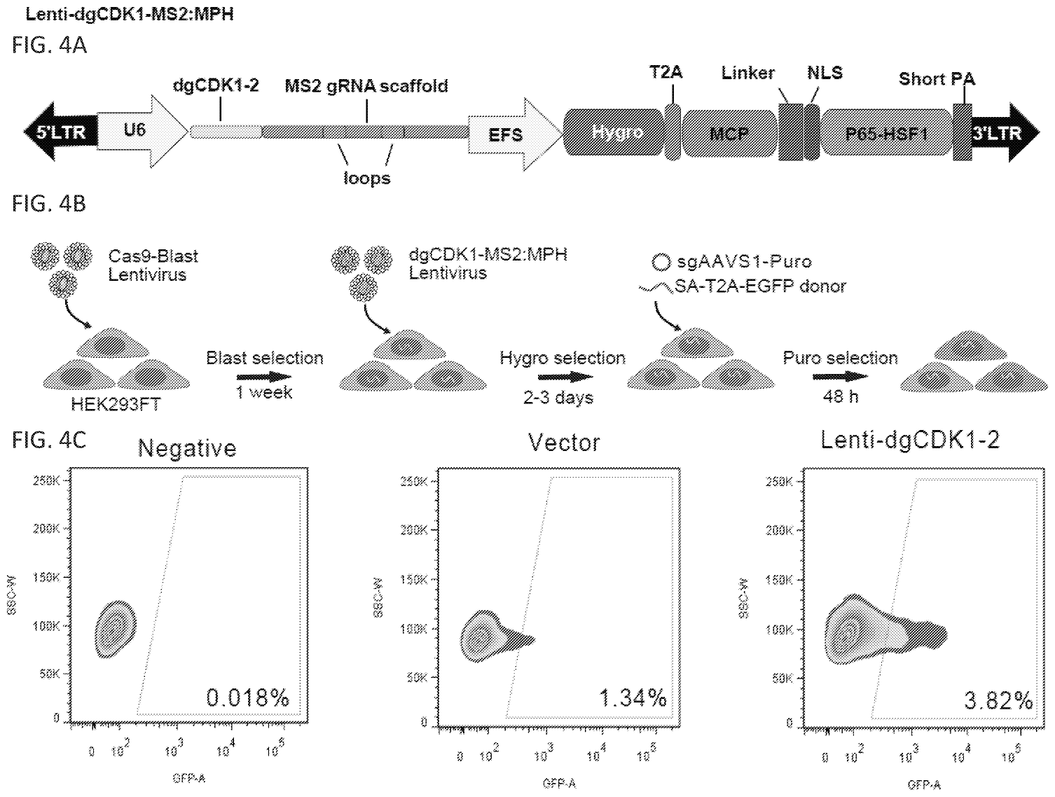

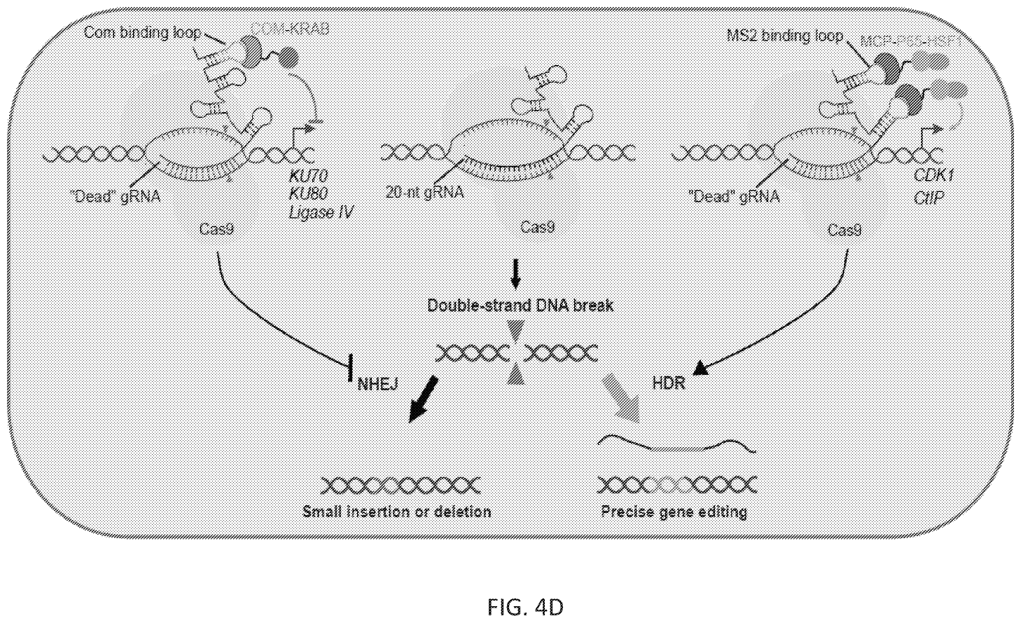

Usage of a lentiviral system was adopted for stable integration of constructs for CRISPRa of DNA repair factors (FIG. 4A). Lentivirus-integrated cell lines expressing dgCDK1-MS2:MPH were generated (SEQ ID NO: 38), and the endogenous AAVS1 targeting experiment was repeated with introduction of an HDR donor and sgAAVS1-Puro by transfection (FIG. 4B). Consistent with previous results herein, FACS analysis again showed significant enhancement of HDR efficiency (FIG. 4C), indicating the adaptability of this DNA repair programming mediated HDR enhancement system to viral delivery vehicles.

In conclusion, the data together showed that CRISPRa/i mediated activation and inhibition of key genes related to DNA damage repair pathways is an effective way to increase the efficiency of HDR for precise genome editing in mammalian cells. With the activation of CDK1 by dgRNA-MS2:MPH and/or repression of KU80 by dgRNA-Com:CK, the HDR efficiency can be enhanced by 4-8 fold. In this system, through combinatorial usage of sgRNA and dgRNA for different purposes, genome editing, gene activation and repression were achieved simultaneously simply with a single Cas9 transgene (FIG. 4D).

The approach described herein is versatile and flexible, with active-Cas9-dgRNA mediating CRISPRa/i programming of DNA repair machinery, where the active Cas9 can still perform its function of generating DSB for HDR-mediated precise gene editing. These components can join force with an armamentarium of other genetic tools such as inducible gene expression modules via simple genetic engineering. Furthermore, the CRISPRa/i constructs can be packaged into viral vectors for efficient delivery into a large repertoire of cell types. For in vivo manipulation, the construction size of CRISPRa/i is slightly larger than traditional approaches used for Cas9-based HDR. Two AAV systems can be used for simultaneous delivery of activation or/and repression components and HDR donor template. Finally, this is a genetic approach of HDR enhancement, and thus can be easily adapted for in vivo settings in time- and tissue-specific manner, which is essential for the application of gene therapy.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A vector comprising a first promoter, a dead guide RNA (dgRNA) comprising a 14-15 base pair (bp) sequence that targets a homology directed repair (HDR) gene and two MS2 binding loops, a second promoter, an MCP sequence, and a P65-HSF1 sequence.

2. The vector of claim 1, wherein the vector comprises SEQ ID NO: 1.

3. The vector of claim 1, wherein the HDR gene is selected from the group consisting of CDK1, CtIP, BRCA1/2, RAD50, and RAD51.

4. The vector of claim 1, wherein the sequence that targets a HDR gene is selected from the group consisting of SEQ ID NOs: 3-12.

5. A vector comprising a first promoter, a dgRNA comprising a 14-15 base pair (bp) sequence that targets a non-homologous end joining (NHEJ) gene and a Com binding loop, a second promoter, a Com sequence, and KRAB sequence.

6. The vector of claim 5, wherein the vector comprises SEQ ID NO: 2.

7. The vector of claim 5, wherein the NHEJ gene is selected from the group consisting of LIG4, KU70 and KU80.

8. The vector of claim 5, wherein the NHEJ sequence is selected from the group consisting consisting of SEQ ID NOs. 13-22.

9. The vector of claim 1, wherein the first promoter comprises a CMV promoter or a U6 promoter and the second promoter comprises a CMV promoter or a U6 promoter.

10. The vector of claim 1, wherein the vector further comprises at least one component selected from the group consisting of an NLS sequence, a linker sequence, a polyA sequence, an SV40 sequence, and an antibiotic resistance sequence.

11. A vector comprising a promoter, a nonfunctional green fluorescent reporter containing a CRISPR targeting site, a self cleaving peptide, and a red fluorescent reporter containing a 2-bp shifted reading frame.

12. The vector of claim 11, wherein the nonfunctional green fluorescent reporter comprises an EGFP variant wherein codons 53-63 are disrupted.

13. The vector of claim 11, wherein the promoter is a CMV promoter.

14. The vector of claim 11, further comprising a SV40 poly (A) signal.

15. The vector of claim 11, wherein the vector comprises the nucleotide sequence of SEQ ID NO: 31 or SEQ ID NO: 32.

16. A composition comprising a cell comprising the vector of claim 11 and a Cas9.

17. The composition of claim 16, wherein the cell is a human embryonic kidney 293 (HEK293) cell.

18. A vector comprising a first promoter, an rtTA sequence, a second promoter, a dead guide RNA (dgRNA) comprising a 14-15 base pair (bp) sequence that targets a homology directed repair (HDR) gene and two MS2 binding loops, a TREG3G promoter sequence, an MCP sequence, and a P65-HSF1 sequence.

19. The vector of claim 18, wherein the vector comprises SEQ ID NO: 29.

20. The vector of claim 18, wherein the HDR gene is selected from the group consisting of CDK1, CtIP, BRCA1/2, RAD50, and RAD51.

21. The vector of claim 18, wherein the sequence that targets a HDR gene is selected from the group consisting of SEQ ID NOs: 3-12.

22. A vector comprising a first promoter sequence, an rtTA sequence, a second promoter, a dgRNA comprising a 14-15 base pair (bp) sequence that targets a non-homologous end joining (NHEJ) gene and a COM binding loop, a TREG3G promoter sequence, a COM sequence, and KRAB sequence.

23. The vector of claim 22, wherein the vector comprises SEQ ID NO: 30.

24. The vector of claim 22, wherein the NHEJ gene is selected from the group consisting of LIG4, KU70 and KU80.

25. The vector of claim 22, wherein the NHEJ sequence is selected from the group consisting of SEQ ID NOs. 13-22.

26. The vector of claim 18, wherein the first promoter comprises a CMV promoter or a U6 promoter and the second promoter comprises a CMV promoter or a U6 promoter.

27. The vector of claim 18, wherein the plasmid further comprises at least one component selected from the group consisting of an NLS sequence, a linker sequence, a polyA sequence, an SV40 sequence, and an antibiotic resistance sequence.

28. A vector comprising a first promoter, a dgRNA comprising a CDK1-2 targeting sequence and and two MS2 binding loops, a second promoter, an MCP sequence, and a P65-HSF1 sequence.

29. The vector of claim 1, wherein the vector comprises a lentiviral backbone.

30. The vector of claim 28, wherein the vector comprises SEQ ID NO: 38.

31. A cell comprising the vector of claim 1.

32. The cell of claim 31, wherein the cell further comprises a Cas9.

33. The cell of claim 31, wherein the cell is an HEK293 cell.

34. A method of enhancing homology directed repair (HDR) and/or decreasing DNA non-homologous end-joining (NHEJ) following CRISPR editing in a cell, the method comprising administering to the cell a Cas9, a sgRNA, an activation plasmid, and a HDR donor template,
wherein the activation plasmid comprises a first promoter, a dead guide RNA (dgRNA) comprising a 14-15 base pair (bp) sequence that targets a homology directed repair (HDR) gene and two MS2 binding loops, a second promoter, an MCP sequence, and a P65-HSF1 sequence.

35. A method of enhancing homology directed repair (HDR) and/or decreasing DNA non-homologous end-joining (NHEJ) following CRISPR editing in a cell, the method comprising administering to the cell a Cas9, a sgRNA, a repression plasmid, and a HDR donor template,
wherein the repression plasmid comprises a first promoter, a dgRNA comprising a 14-15 base pair (bp) sequence that targets a non-homologous end joining (NHEJ) gene and a Com binding loop, a second promoter, a Com sequence, and KRAB sequence.

36. A method of enhancing homology directed repair (HDR) and/or decreasing DNA non-homologous end-joining (NHEJ) following CRISPR editing in a cell, the method comprising administering to the cell a Cas9, a sgRNA, an activation plasmid, a repression plasmid, and a HDR donor template,
wherein the activation plasmid comprises a first promoter, a dead guide RNA (dgRNA) comprising a 14-15 base pair (bp) sequence that targets a homology directed repair (HDR) gene and two MS2 binding loops, a second promoter, an MCP sequence, and a P65-HSF1 sequence, and
wherein the repression plasmid comprises a first promoter, a dgRNA comprising a 14-15 base pair (bp) sequence that targets a non-homologous end joining (NHEJ) gene and a Com binding loop, a second promoter, a Com sequence, and KRAB sequence.

37. The method of claim 34, wherein the activation plasmid targets CDK1-2 and/or the repression plasmid targets KU80-1.

38. The method of claim 34, wherein the repression and/or activation plasmid further comprises an inducible expression system.

39. The method of claim 38, wherein the inducible expression system is a Tet-On system inducible by doxycycline (Dox).

40. The method of claim 34, wherein the activation plasmid comprises SEQ ID NO: 1.

41. The method of claim 34, wherein the HDR gene is selected from the group consisting of CDK1, CtIP, BRCA1/2, RAD50, and RAD51.

42. The method of claim 34, wherein the sequence that targets a HDR gene is selected from the group consisting of SEQ ID NOs: 3-12.

43. The method of claim 35, wherein the repression plasmid comprises SEQ ID NO: 2.

44. The method of claim 35, wherein the NHEJ gene is selected from the group consisting of LIG4, KU70 and KU80.

45. The method of claim 35, wherein the NHEJ sequence is selected from the group consisting of SEQ ID NOs. 13-22.

46. The method of claim 34, wherein the first promoter of the repression and/or activation plasmid comprises a CMV promoter or a U6 promoter and the second promoter of the repression and/or activation plasmid comprises a CMV promoter or a U6 promoter.

47. The method of claim 34, wherein the repression and/or activation plasmid further comprises at least one component selected from the group consisting of an NLS sequence, a linker sequence, a polyA sequence, an SV40 sequence, and an antibiotic resistance sequence.

48. The method of claim 34, further comprising administering the cell to an animal.

49. The method of claim 34, wherein the repression and/or activation plasmid is packaged into a lentiviral vector.

50. The method of claim 49, further comprising administering the lentiviral vector to an animal.

51. The method of claim 48, wherein the animal is a human.

52. A composition comprising the vector of claim 1.

53. A composition comprising the vector of claim 18.

54. The composition of claim 52, further comprising a Cas9.

55. A kit comprising the vector of claim 1, and instructional material for use thereof.

56. A kit comprising the vector of claim 18, and instructional material for use thereof.

57. The kit of claim 55, further comprising a Cas9.
